SEQUENCE LISTING (246 sequences)

SEQ ID NO: 1 - protein, 5 aa, Synthetic construct
EYTIH

SEQ ID NO: 2 - protein, 17 aa, Synthetic construct
NINPNNGGTTYNQKFED

SEQ ID NO: 3 - protein, 6 aa, Synthetic construct
GWNFDY

SEQ ID NO: 4 - protein, 115 aa, Synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSS

SEQ ID NO: 5 - DNA, 15 bp, Synthetic construct
  source 1..15: mol_type = other DNA
gaatacaccatccac

SEQ ID NO: 6 - DNA, 51 bp, Synthetic construct
  source 1..51: mol_type = other DNA
aacattaatcctaacaatggtggtactacctacaaccagaagttcgaggac

SEQ ID NO: 7 - DNA, 18 bp, Synthetic construct
  source 1..18: mol_type = other DNA
ggttggaactttgactac

SEQ ID NO: 8 - DNA, 345 bp, Synthetic construct
  source 1..345: mol_type = other DNA
gaggtccagctggtgcagtctggagctgaggtgaagaagcctggggcctcagtgaaggtctcctgcaaggcttctggatacacattcactgaatacaccatccactgggtgaggcaggcccctggaaagggccttgagtggattggaaacattaatcctaacaatggtggtactacctacaaccagaagttcgaggacagagtcacaatcactgtagacaagtccaccagcacagcctacatggagctcagcagcctgagatctgaggatactgcagtctattactgtgcagctggttggaactttgactactggggccaaggcaccacggtcaccgtctcctca

SEQ ID NO: 9 - protein, 30 aa, Synthetic construct
EVQLVQSGAEVKKPGASVKVSCKASGYTFT

SEQ ID NO: 10 - protein, 14 aa, Synthetic construct
WVRQAPGKGLEWIG

SEQ ID NO: 11 - protein, 32 aa, Synthetic construct
RVTITVDKSTSTAYMELSSLRSEDTAVYYCAA

SEQ ID NO: 12 - protein, 11 aa, Synthetic construct
WGQGTTVTVSS

SEQ ID NO: 13 - DNA, 90 bp, Synthetic construct
  source 1..90: mol_type = other DNA
gaggtccagctggtgcagtctggagctgaggtgaagaagcctggggcctcagtgaaggtctcctgcaaggcttctggatacacattcact

SEQ ID NO: 14 - DNA, 42 bp, Synthetic construct
  source 1..42: mol_type = other DNA
tgggtgaggcaggcccctggaaagggccttgagtggattgga

SEQ ID NO: 15 - DNA, 96 bp, Synthetic construct
  source 1..96: mol_type = other DNA
agagtcacaatcactgtagacaagtccaccagcacagcctacatggagctcagcagcctgagatctgaggatactgcagtctattactgtgcagct

SEQ ID NO: 16 - DNA, 33 bp, Synthetic construct
  source 1..33: mol_type = other DNA
tggggccaaggcaccacggtcaccgtctcctca

SEQ ID NO: 17 - protein, 5 aa, Synthetic construct
EYTIH

SEQ ID NO: 18 - protein, 17 aa, Synthetic construct
NINPNNGGTTYNQKFED

SEQ ID NO: 19 - protein, 6 aa, Synthetic construct
YWLFDY

SEQ ID NO: 20 - protein, 115 aa, Synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAYWLFDYWGQGTTVTVSS

SEQ ID NO: 21 - DNA, 15 bp, Synthetic construct
  source 1..15: mol_type = other DNA
gaatacaccatccac

SEQ ID NO: 22 - DNA, 51 bp, Synthetic construct; identical to SEQ ID NO: 6
  source 1..51: mol_type = other DNA

SEQ ID NO: 23 - DNA, 18 bp, Synthetic construct
  source 1..18: mol_type = other DNA
tactggctgttcgactac

SEQ ID NO: 24 - DNA, 345 bp, Synthetic construct
  source 1..345: mol_type = other DNA
gaggtccagctggtgcagtctggagctgaggtgaagaagcctggggcctcagtgaaggtctcctgcaaggcttctggatacacattcactgaatacaccatccactgggtgaggcaggcccctggaaagggccttgagtggattggaaacattaatcctaacaatggtggtactacctacaaccagaagttcgaggacagagtcacaatcactgtagacaagtccaccagcacagcctacatggagctcagcagcctgagatctgaggatactgcagtctattactgtgcagcttactggctgttcgactactggggccaaggcaccacggtcaccgtctcctca

SEQ ID NO: 25 - protein, 30 aa, Synthetic construct; identical to SEQ ID NO: 9

SEQ ID NO: 26 - protein, 14 aa, Synthetic construct
WVRQAPGKGLEWIG

SEQ ID NO: 27 - protein, 32 aa, Synthetic construct; identical to SEQ ID NO: 11

SEQ ID NO: 28 - protein, 11 aa, Synthetic construct
WGQGTTVTVSS

SEQ ID NO: 29 - DNA, 90 bp, Synthetic construct; identical to SEQ ID NO: 13
  source 1..90: mol_type = other DNA

SEQ ID NO: 30 - DNA, 42 bp, Synthetic construct; identical to SEQ ID NO: 14
  source 1..42: mol_type = other DNA

SEQ ID NO: 31 - DNA, 96 bp, Synthetic construct; identical to SEQ ID NO: 15
  source 1..96: mol_type = other DNA

SEQ ID NO: 32 - DNA, 33 bp, Synthetic construct; identical to SEQ ID NO: 16
  source 1..33: mol_type = other DNA

SEQ ID NO: 33 - protein, 11 aa, Synthetic construct
KASQDVGTAVD

SEQ ID NO: 34 - protein, 7 aa, Synthetic construct
WASTRHT

SEQ ID NO: 35 - protein, 9 aa, Synthetic construct
QQYNSYPLT

SEQ ID NO: 36 - protein, 107 aa, Synthetic construct
DIQMTQSPSTLSASVGDRVTITCKASQDVGTAVDWYQQKPGQAPKLLIYWASTRHTGVPDRFSGSGSGTDFTLTISRLQPEDFAVYYCQQYNSYPLTFGQGTKVDIK

SEQ ID NO: 37 - DNA, 33 bp, Synthetic construct
  source 1..33: mol_type = other DNA
aaggccagtcaggatgtgggtactgctgtagac

SEQ ID NO: 38 - DNA, 21 bp, Synthetic construct
  source 1..21: mol_type = other DNA
tgggcatccacccggcacact

SEQ ID NO: 39 - DNA, 27 bp, Synthetic construct
  source 1..27: mol_type = other DNA
cagcaatataacagctatcctctcacg

SEQ ID NO: 40 - DNA, 321 bp, Synthetic construct
  source 1..321: mol_type = other DNA
gacattcagatgacccagtctcccagcaccctgtccgcatcagtaggagacagggtcaccatcacttgcaaggccagtcaggatgtgggtactgctgtagactggtatcaacagaaaccagggcaagctcctaaactactgatttactgggcatccacccggcacactggagtccctgatcgcttcagcggcagtggatctgggacagatttcactctcaccatcagcagactgcagcctgaagactttgcagtttattactgtcagcaatataacagctatcctctcacgttcggccaggggaccaaggtggatatcaaa

SEQ ID NO: 41 - protein, 23 aa, Synthetic construct
DIQMTQSPSTLSASVGDRVTITC

SEQ ID NO: 42 - protein, 15 aa, Synthetic construct
WYQQKPGQAPKLLIY

SEQ ID NO: 43 - protein, 32 aa, Synthetic construct
GVPDRFSGSGSGTDFTLTISRLQPEDFAVYYC

SEQ ID NO: 44 - protein, 10 aa, Synthetic construct
FGQGTKVDIK

SEQ ID NO: 45 - DNA, 69 bp, Synthetic construct
  source 1..69: mol_type = other DNA
gacattcagatgacccagtctcccagcaccctgtccgcatcagtaggagacagggtcaccatcacttgc

SEQ ID NO: 46 - DNA, 45 bp, Synthetic construct
  source 1..45: mol_type = other DNA
tggtatcaacagaaaccagggcaagctcctaaactactgatttac

SEQ ID NO: 47 - DNA, 96 bp, Synthetic construct
  source 1..96: mol_type = other DNA
ggagtccctgatcgcttcagcggcagtggatctgggacagatttcactctcaccatcagcagactgcagcctgaagactttgcagtttattactgt

SEQ ID NO: 48 - DNA, 30 bp, Synthetic construct
  source 1..30: mol_type = other DNA
ttcggccaggggaccaaggtggatatcaaa

SEQ ID NO: 49 - protein, 5 aa, Synthetic construct
NYYMS

SEQ ID NO: 50 - protein, 17 aa, Synthetic construct
AINSDGGITYYLDTVKG

SEQ ID NO: 51 - protein, 10 aa, Synthetic construct
HRSGYFSMDY

SEQ ID NO: 52 - protein, 119 aa, Synthetic construct
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSS

SEQ ID NO: 53 - DNA, 15 bp, Synthetic construct
  source 1..15: mol_type = other DNA
aactattacatgtct

SEQ ID NO: 54 - DNA, 51 bp, Synthetic construct
  source 1..51: mol_type = other DNA
gccattaatagtgatggtggtatcacctactatctagacactgtgaagggc

SEQ ID NO: 55 - DNA, 30 bp, Synthetic construct
  source 1..30: mol_type = other DNA
caccgctcgggctacttttctatggactac

SEQ ID NO: 56 - DNA, 357 bp, Synthetic construct
  source 1..357: mol_type = other DNA
gacgtgaagctcgtggagtctgggggaggcttagtgaagcttggaggatccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctca

SEQ ID NO: 57 - protein, 30 aa, Synthetic construct
DVKLVESGGGLVKLGGSLKLSCAASGFTFS

SEQ ID NO: 58 - protein, 14 aa, Synthetic construct
WVRQTPEKRLELVA

SEQ ID NO: 59 - protein, 32 aa, Synthetic construct
RFTISRDNAKNTLYLQMSSLKSEDTALFYCAR

SEQ ID NO: 60 - protein, 11 aa, Synthetic construct
WGQGTSVTVSS

SEQ ID NO: 61 - DNA, 90 bp, Synthetic construct
  source 1..90: mol_type = other DNA
gacgtgaagctcgtggagtctgggggaggcttagtgaagcttggaggatccctgaaactctcctgtgcagcctctggattcactttcagt

SEQ ID NO: 62 - DNA, 42 bp, Synthetic construct
  source 1..42: mol_type = other DNA
tgggttcgccagactccagagaagaggctggagttggtcgca

SEQ ID NO: 63 - DNA, 96 bp, Synthetic construct
  source 1..96: mol_type = other DNA
cgattcaccatttcaagagacaatgccaagaacaccctgtacctgcaaatgagcagtctgaagtctgaggacacagccttgttttactgtgcaaga

SEQ ID NO: 64 - DNA, 33 bp, Synthetic construct
  source 1..33: mol_type = other DNA
tggggtcaaggaacctcagtcaccgtctcctca

SEQ ID NO: 65 - protein, 5 aa, Synthetic construct
NYYMS

SEQ ID NO: 66 - protein, 17 aa, Synthetic construct
AINSDGGITYYLDTVKG

SEQ ID NO: 67 - protein, 10 aa, Synthetic construct
HRSGYFSMDY

SEQ ID NO: 68 - protein, 119 aa, Synthetic construct
DVKLVESGGGLVKPGGSLRLSCAASGFTFSNYYMSWVRQAPGKGLELVAAINSDGGITYYLDTVKGRFTISRDNAKNTLYLQMSSLKSEDTALYYCARHRSGYFSMDYWGQGTSVTVSS

SEQ ID NO: 69 - DNA, 15 bp, Synthetic construct
  source 1..15: mol_type = other DNA
aactactacatgagc

SEQ ID NO: 70 - DNA, 51 bp, Synthetic construct
  source 1..51: mol_type = other DNA
gccattaacagtgacggtggcatcacctactacctggacaccgtgaagggc

SEQ ID NO: 71 - DNA, 30 bp, Synthetic construct
  source 1..30: mol_type = other DNA
cacaggagcggctacttctctatggactac

SEQ ID NO: 72 - DNA, 357 bp, Synthetic construct
  source 1..357: mol_type = other DNA
gacgtgaagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgagcagcctgaagagcgaggacacggccctgtattactgtgcgagacacaggagcggctacttctctatggactactggggccagggcaccagcgtcactgtctcctca

SEQ ID NO: 73 - protein, 30 aa, Synthetic construct
DVKLVESGGGLVKPGGSLRLSCAASGFTFS

SEQ ID NO: 74 - protein, 14 aa, Synthetic construct
WVRQAPGKGLELVA

SEQ ID NO: 75 - protein, 32 aa, Synthetic construct
RFTISRDNAKNTLYLQMSSLKSEDTALYYCAR

SEQ ID NO: 76 - protein, 11 aa, Synthetic construct
WGQGTSVTVSS

SEQ ID NO: 77 - DNA, 90 bp, Synthetic construct
  source 1..90: mol_type = other DNA
gacgtgaagctggtggagtctgggggaggcttggtcaagcctggaggatccctgagactctcctgtgcagcctctggattcaccttcagt

SEQ ID NO: 78 - DNA, 42 bp, Synthetic construct
  source 1..42: mol_type = other DNA
tgggtgcgccaggctccagggaaggggctggagctggttgcc

SEQ ID NO: 79 - DNA, 96 bp, Synthetic construct
  source 1..96: mol_type = other DNA
cgattcaccatctccagggacaacgccaagaacaccctgtatctgcaaatgagcagcctgaagagcgaggacacggccctgtattactgtgcgaga

SEQ ID NO: 80 - DNA, 33 bp, Synthetic construct
  source 1..33: mol_type = other DNA
tggggccagggcaccagcgtcactgtctcctca

SEQ ID NO: 81 - protein, 5 aa, Synthetic construct
NYYMS

SEQ ID NO: 82 - protein, 17 aa, Synthetic construct
AINSDGGITYYLDTVKG

SEQ ID NO: 83 - protein, 10 aa, Synthetic construct
HRSGYFSMDY

SEQ ID NO: 84 - protein, 119 aa, Synthetic construct
DVKLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSS

SEQ ID NO: 85 - DNA, 15 bp, Synthetic construct
  source 1..15: mol_type = other DNA
aactactacatgagc

SEQ ID NO: 86 - DNA, 50 bp, Synthetic construct
  source 1..50: mol_type = other DNA
ccattaacagtgacggtggcatcacctactacctggacaccgtgaagggc

SEQ ID NO: 87 - DNA, 30 bp, Synthetic construct; identical to SEQ ID NO: 71
  source 1..30: mol_type = other DNA

SEQ ID NO: 88 - DNA, 357 bp, Synthetic construct
  source 1..357: mol_type = other DNA
gacgtgaagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 89 - protein, 30 aa, Synthetic construct; identical to SEQ ID NO: 73

SEQ ID NO: 90 - protein, 14 aa, Synthetic construct
WVRQAPGKGLELVA

SEQ ID NO: 91 - protein, 32 aa, Synthetic construct
RFTISRDNAKNTLYLQMNSLRAEDTALYYCAR

SEQ ID NO: 92 - protein, 11 aa, Synthetic construct
WGQGTLVTVSS

SEQ ID NO: 93 - DNA, 90 bp, Synthetic construct; identical to SEQ ID NO: 77
  source 1..90: mol_type = other DNA

SEQ ID NO: 94 - DNA, 42 bp, Synthetic construct; identical to SEQ ID NO: 78
  source 1..42: mol_type = other DNA

SEQ ID NO: 95 - DNA, 96 bp, Synthetic construct
  source 1..96: mol_type = other DNA
cgattcaccatctccagggacaacgccaagaacaccctgtatctgcaaatgaacagcctgagggccgaggacacggccctgtattactgtgcgaga

SEQ ID NO: 96 - DNA, 33 bp, Synthetic construct
  source 1..33: mol_type = other DNA
tggggccagggcaccctggtcactgtctcctca

SEQ ID NO: 97 - protein, 5 aa, Synthetic construct
NYYMS

SEQ ID NO: 98 - protein, 17 aa, Synthetic construct
AINSDGGITYYLDTVKG

SEQ ID NO: 99 - protein, 10 aa, Synthetic construct
HRSGYFSMDY

SEQ ID NO: 100 - protein, 119 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSS

SEQ ID NO: 101 - DNA, 15 bp, Synthetic construct
  source 1..15: mol_type = other DNA
aactactacatgagc

SEQ ID NO: 102 - DNA, 51 bp, Synthetic construct; identical to SEQ ID NO: 70
  source 1..51: mol_type = other DNA

SEQ ID NO: 103 - DNA, 30 bp, Synthetic construct; identical to SEQ ID NO: 71
  source 1..30: mol_type = other DNA

SEQ ID NO: 104 - DNA, 357 bp, Synthetic construct
  source 1..357: mol_type = other DNA
gaggtgcagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 105 - protein, 30 aa, Synthetic construct
EVQLVESGGGLVKPGGSLRLSCAASGFTFS

SEQ ID NO: 106 - protein, 14 aa, Synthetic construct
WVRQAPGKGLELVA

SEQ ID NO: 107 - protein, 32 aa, Synthetic construct; identical to SEQ ID NO: 91

SEQ ID NO: 108 - protein, 11 aa, Synthetic construct
WGQGTLVTVSS

SEQ ID NO: 109 - DNA, 90 bp, Synthetic construct
  source 1..90: mol_type = other DNA
gaggtgcagctggtggagtctgggggaggcttggtcaagcctggaggatccctgagactctcctgtgcagcctctggattcaccttcagt

SEQ ID NO: 110 - DNA, 42 bp, Synthetic construct; identical to SEQ ID NO: 78
  source 1..42: mol_type = other DNA

SEQ ID NO: 111 - DNA, 96 bp, Synthetic construct; identical to SEQ ID NO: 95
  source 1..96: mol_type = other DNA

SEQ ID NO: 112 - DNA, 33 bp, Synthetic construct; identical to SEQ ID NO: 96
  source 1..33: mol_type = other DNA

SEQ ID NO: 113 - protein, 5 aa, Synthetic construct
NYYMS

SEQ ID NO: 114 - protein, 17 aa, Synthetic construct
AINSDGGITYYLDTVKG

SEQ ID NO: 115 - protein, 10 aa, Synthetic construct
HRSGYFSMDY

SEQ ID NO: 116 - protein, 119 aa, Synthetic construct
EVQLVESGGGLVKPGGSLRLSCAASGFTFSNYYMSWVRQAPGKGLEWVAAINSDGGITYYLDTVKGRFTISRDNAKNTLYLQMNSLRAEDTALYYCARHRSGYFSMDYWGQGTLVTVSS

SEQ ID NO: 117 - DNA, 15 bp, Synthetic construct
  source 1..15: mol_type = other DNA
aactactacatgagc

SEQ ID NO: 118 - DNA, 51 bp, Synthetic construct; identical to SEQ ID NO: 70
  source 1..51: mol_type = other DNA

SEQ ID NO: 119 - DNA, 30 bp, Synthetic construct; identical to SEQ ID NO: 71
  source 1..30: mol_type = other DNA

SEQ ID NO: 120 - DNA, 357 bp, Synthetic construct
  source 1..357: mol_type = other DNA
gaggtgcagctggtggagtctgggggaggcttggtcaagcctggaggatccctgagactctcctgtgcagcctctggattcaccttcagtaactactacatgagctgggtgcgccaggctccagggaaggggctggagtgggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 121 - protein, 30 aa, Synthetic construct; identical to SEQ ID NO: 105

SEQ ID NO: 122 - protein, 14 aa, Synthetic construct
WVRQAPGKGLEWVA

SEQ ID NO: 123 - protein, 32 aa, Synthetic construct; identical to SEQ ID NO: 91

SEQ ID NO: 124 - protein, 11 aa, Synthetic construct
WGQGTLVTVSS

SEQ ID NO: 125 - DNA, 90 bp, Synthetic construct; identical to SEQ ID NO: 109
  source 1..90: mol_type = other DNA

SEQ ID NO: 126 - DNA, 42 bp, Synthetic construct
  source 1..42: mol_type = other DNA
tgggtgcgccaggctccagggaaggggctggagtgggttgcc

SEQ ID NO: 127 - DNA, 96 bp, Synthetic construct; identical to SEQ ID NO: 95
  source 1..96: mol_type = other DNA

SEQ ID NO: 128 - DNA, 33 bp, Synthetic construct; identical to SEQ ID NO: 96
  source 1..33: mol_type = other DNA

SEQ ID NO: 129 - protein, 11 aa, Synthetic construct
KASQNVVSAVA

SEQ ID NO: 130 - protein, 7 aa, Synthetic construct
SASNRYT

SEQ ID NO: 131 - protein, 9 aa, Synthetic construct
QQYSNYPWT

SEQ ID NO: 132 - protein, 107 aa, Synthetic construct
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIK

SEQ ID NO: 133 - DNA, 33 bp, Synthetic construct
  source 1..33: mol_type = other DNA
aaggccagtcagaatgtggtttctgctgttgcc

SEQ ID NO: 134 - DNA, 21 bp, Synthetic construct
  source 1..21: mol_type = other DNA
tcagcatccaatcggtacact

SEQ ID NO: 135 - DNA, 27 bp, Synthetic construct
  source 1..27: mol_type = other DNA
caacaatatagcaactatccgtggacg

SEQ ID NO: 136 - DNA, 321 bp, Synthetic construct
  source 1..321: mol_type = other DNA
gacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaa

SEQ ID NO: 137 - protein, 23 aa, Synthetic construct
DIVMTQSQRFMSTTVGDRVSITC

SEQ ID NO: 138 - protein, 15 aa, Synthetic construct
WYQQKPGQSPKLLIY

SEQ ID NO: 139 - protein, 32 aa, Synthetic construct
GVPDRFTGSGSGTDFTLTISNMQSEDLADFFC

SEQ ID NO: 140 - protein, 10 aa, Synthetic construct
FGGGTKLEIK

SEQ ID NO: 141 - DNA, 69 bp, Synthetic construct
  source 1..69: mol_type = other DNA
gacattgtgatgacccagtctcaaagattcatgtccacaacagtaggagacagggtcagcatcacctgc

SEQ ID NO: 142 - DNA, 45 bp, Synthetic construct
  source 1..45: mol_type = other DNA
tggtatcaacagaaaccaggacaatctcctaaactactgatttac

SEQ ID NO: 143 - DNA, 96 bp, Synthetic construct
  source 1..96: mol_type = other DNA
ggagtccctgatcgcttcacaggcagtggatctgggacagatttcactctcaccattagcaatatgcagtctgaagacctggctgattttttctgt

SEQ ID NO: 144 - DNA, 30 bp, Synthetic construct
  source 1..30: mol_type = other DNA
ttcggtggaggcaccaagctggaaatcaaa

SEQ ID NO: 145 - protein, 11 aa, Synthetic construct
KASQNVVSAVA

SEQ ID NO: 146 - protein, 7 aa, Synthetic construct
SASNRYT

SEQ ID NO: 147 - protein, 9 aa, Synthetic construct
QQYSNYPWT

SEQ ID NO: 148 - protein, 107 aa, Synthetic construct
DIVMTQSPSFLSASVGDRVTITCKASQNVVSAVAWYQQKPGQAPKLLIYSASNRYTGVPDRFTGSGSGTDFTLTISSLQSEDLADYFCQQYSNYPWTFGGGTKVEIK

SEQ ID NO: 149 - DNA, 33 bp, Synthetic construct
  source 1..33: mol_type = other DNA
aaggcaagtcagaacgtggtgagtgctgtggcc

SEQ ID NO: 150 - DNA, 21 bp, Synthetic construct
  source 1..21: mol_type = other DNA
agcgcctccaacaggtacacc

SEQ ID NO: 151 - DNA, 27 bp, Synthetic construct
  source 1..27: mol_type = other DNA
caacagtacagcaattacccttggacg

SEQ ID NO: 152 - DNA, 321 bp, Synthetic construct
  source 1..321: mol_type = other DNA
gacatcgtgatgacccagtctccatccttcctgtctgcatctgtaggagacagagtcaccatcacttgcaaggcaagtcagaacgtggtgagtgctgtggcctggtatcagcagaaaccagggcaggctcctaagctcctgatctatagcgcctccaacaggtacaccggggtcccagacaggttcaccggcagtggatctgggacagatttcactctcaccatcagcagcctgcagagcgaagatctggcagactatttctgtcaacagtacagcaattacccttggacgttcggcggcgggaccaaggtggaaatcaaa

SEQ ID NO: 153 - protein, 23 aa, Synthetic construct
DIVMTQSPSFLSASVGDRVTITC

SEQ ID NO: 154 - protein, 15 aa, Synthetic construct
WYQQKPGQAPKLLIY

SEQ ID NO: 155 - protein, 32 aa, Synthetic construct
GVPDRFTGSGSGTDFTLTISSLQSEDLADYFC

SEQ ID NO: 156 - protein, 10 aa, Synthetic construct
FGGGTKVEIK

SEQ ID NO: 157 - DNA, 69 bp, Synthetic construct
  source 1..69: mol_type = other DNA
gacatcgtgatgacccagtctccatccttcctgtctgcatctgtaggagacagagtcaccatcacttgc

SEQ ID NO: 158 - DNA, 45 bp, Synthetic construct
  source 1..45: mol_type = other DNA
tggtatcagcagaaaccagggcaggctcctaagctcctgatctat

SEQ ID NO: 159 - DNA, 96 bp, Synthetic construct
  source 1..96: mol_type = other DNA
ggggtcccagacaggttcaccggcagtggatctgggacagatttcactctcaccatcagcagcctgcagagcgaagatctggcagactatttctgt

SEQ ID NO: 160 - DNA, 30 bp, Synthetic construct
  source 1..30: mol_type = other DNA
ttcggcggcgggaccaaggtggaaatcaaa

SEQ ID NO: 161 - protein, 11 aa, Synthetic construct
KASQNVVSAVA

SEQ ID NO: 162 - protein, 7 aa, Synthetic construct
SASNRYT

SEQ ID NO: 163 - protein, 9 aa, Synthetic construct
QQYSNYPWT

SEQ ID NO: 164 - protein, 107 aa, Synthetic construct
DIVMTQSPSSLSASVGDRVTITCKASQNVVSAVAWYQQKPGQAPRLLIYSASNRYTGVPDRFTGSGSGTDFTLTISSLQAEDLADYFCQQYSNYPWTFGGGTKVEIK

SEQ ID NO: 165 - DNA, 33 bp, Synthetic construct; identical to SEQ ID NO: 149
  source 1..33: mol_type = other DNA

SEQ ID NO: 166 - DNA, 21 bp, Synthetic construct
  source 1..21: mol_type = other DNA
agcgcctccaacaggtacacc

SEQ ID NO: 167 - DNA, 27 bp, Synthetic construct
  source 1..27: mol_type = other DNA
caacagtacagcaattacccttggacg

SEQ ID NO: 168 - DNA, 321 bp, Synthetic construct
  source 1..321: mol_type = other DNA
gacatcgtgatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcaccggcagtggatctgggacagatttcactctcaccatcagcagcctgcaggccgaagatctggcagactatttctgtcaacagtacagcaattacccttggacgttcggcggcgggaccaaggtggaaatcaaa

SEQ ID NO: 169 - protein, 23 aa, Synthetic construct
DIVMTQSPSSLSASVGDRVTITC

SEQ ID NO: 170 - protein, 15 aa, Synthetic construct
WYQQKPGQAPRLLIY

SEQ ID NO: 171 - protein, 32 aa, Synthetic construct
GVPDRFTGSGSGTDFTLTISSLQAEDLADYFC

SEQ ID NO: 172 - protein, 10 aa, Synthetic construct
FGGGTKVEIK

SEQ ID NO: 173 - DNA, 69 bp, Synthetic construct
  source 1..69: mol_type = other DNA
gacatcgtgatgacccagtctccatccagcctgtctgcatctgtaggagacagagtcaccatcacttgc

SEQ ID NO: 174 - DNA, 45 bp, Synthetic construct
  source 1..45: mol_type = other DNA
tggtatcagcagaaaccagggcaggctcctaggctcctgatctat

SEQ ID NO: 175 - DNA, 96 bp, Synthetic construct
  source 1..96: mol_type = other DNA
ggggtcccagacaggttcaccggcagtggatctgggacagatttcactctcaccatcagcagcctgcaggccgaagatctggcagactatttctgt

SEQ ID NO: 176 - DNA, 30 bp, Synthetic construct; identical to SEQ ID NO: 160
  source 1..30: mol_type = other DNA

SEQ ID NO: 177 - protein, 11 aa, Synthetic construct
KASQNVVSAVA

SEQ ID NO: 178 - protein, 7 aa, Synthetic construct
SASNRYT

SEQ ID NO: 179 - protein, 9 aa, Synthetic construct
QQYSNYPWT

SEQ ID NO: 180 - protein, 107 aa, Synthetic construct
DIQMTQSPSSLSASVGDRVTITCKASQNVVSAVAWYQQKPGQAPRLLIYSASNRYTGVPDRFSGSGSGTDFTLTISSLQAEDLADYFCQQYSNYPWTFGGGTKVEIK

SEQ ID NO: 181 - DNA, 33 bp, Synthetic construct; identical to SEQ ID NO: 149
  source 1..33: mol_type = other DNA

SEQ ID NO: 182 - DNA, 21 bp, Synthetic construct
  source 1..21: mol_type = other DNA
agcgcctccaacaggtacacc

SEQ ID NO: 183 - DNA, 27 bp, Synthetic construct
  source 1..27: mol_type = other DNA
caacagtacagcaattacccttggacg

SEQ ID NO: 184 - DNA, 321 bp, Synthetic construct
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcagcggcagtggatctgggacagatttcactctcaccatcagcagcctgcaggccgaagatctggcagactatttctgtcaacagtacagcaattacccttggacgttcggcggcgggaccaaggtggaaatcaaa

SEQ ID NO: 185 - protein, 23 aa, Synthetic construct
DIQMTQSPSSLSASVGDRVTITC

SEQ ID NO: 186 - protein, 15 aa, Synthetic construct
WYQQKPGQAPRLLIY

SEQ ID NO: 187 - protein, 32 aa, Synthetic construct
GVPDRFSGSGSGTDFTLTISSLQAEDLADYFC

SEQ ID NO: 188 - protein, 10 aa, Synthetic construct
FGGGTKVEIK

SEQ ID NO: 189 - DNA, 69 bp, Synthetic construct
  source 1..69: mol_type = other DNA
gacatccagatgacccagtctccatccagcctgtctgcatctgtaggagacagagtcaccatcacttgc

SEQ ID NO: 190 - DNA, 45 bp, Synthetic construct; identical to SEQ ID NO: 174
  source 1..45: mol_type = other DNA

SEQ ID NO: 191 - DNA, 96 bp, Synthetic construct
  source 1..96: mol_type = other DNA
ggggtcccagacaggttcagcggcagtggatctgggacagatttcactctcaccatcagcagcctgcaggccgaagatctggcagactatttctgt

SEQ ID NO: 192 - DNA, 30 bp, Synthetic construct; identical to SEQ ID NO: 160
  source 1..30: mol_type = other DNA

SEQ ID NO: 193 - protein, 11 aa, Synthetic construct
KASQNVVSAVA

SEQ ID NO: 194 - protein, 7 aa, Synthetic construct
SASNRYT

SEQ ID NO: 195 - protein, 9 aa, Synthetic construct
QQYSNYPWT

SEQ ID NO: 196 - protein, 107 aa, Synthetic construct
DIQMTQSPSSLSASVGDRVTITCKASQNVVSAVAWYQQKPGQAPRLLIYSASNRYTGVPDRFSGSGSGTDFTLTISSLQAEDLADYYCQQYSNYPWTFGGGTKVEIK

SEQ ID NO: 197 - DNA, 33 bp, Synthetic construct; identical to SEQ ID NO: 149
  source 1..33: mol_type = other DNA

SEQ ID NO: 198 - DNA, 21 bp, Synthetic construct
  source 1..21: mol_type = other DNA
agcgcctccaacaggtacacc

SEQ ID NO: 199 - DNA, 27 bp, Synthetic construct
  source 1..27: mol_type = other DNA
caacagtacagcaattacccttggacg

SEQ ID NO: 200 - DNA, 321 bp, Synthetic construct
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcagcggcagtggatctgggacagatttcactctcaccatcagcagcctgcaggccgaagatctggcagactattactgtcaacagtacagcaattacccttggacgttcggcggcgggaccaaggtggaaatcaaa

SEQ ID NO: 201 - protein, 23 aa, Synthetic construct
DIQMTQSPSSLSASVGDRVTITC

SEQ ID NO: 202 - protein, 15 aa, Synthetic construct
WYQQKPGQAPRLLIY

SEQ ID NO: 203 - protein, 32 aa, Synthetic construct
GVPDRFSGSGSGTDFTLTISSLQAEDLADYYC

SEQ ID NO: 204 - protein, 10 aa, Synthetic construct
FGGGTKVEIK

SEQ ID NO: 205 - DNA, 69 bp, Synthetic construct; identical to SEQ ID NO: 189
  source 1..69: mol_type = other DNA

SEQ ID NO: 206 - DNA, 45 bp, Synthetic construct; identical to SEQ ID NO: 174
  source 1..45: mol_type = other DNA

SEQ ID NO: 207 - DNA, 96 bp, Synthetic construct
  source 1..96: mol_type = other DNA
ggggtcccagacaggttcagcggcagtggatctgggacagatttcactctcaccatcagcagcctgcaggccgaagatctggcagactattactgt

SEQ ID NO: 208 - DNA, 30 bp, Synthetic construct; identical to SEQ ID NO: 160
  source 1..30: mol_type = other DNA

SEQ ID NO: 209 - protein, 11 aa, Synthetic construct
KASQNVVSAVA

SEQ ID NO: 210 - protein, 7 aa, Synthetic construct
SASNRYT

SEQ ID NO: 211 - protein, 9 aa, Synthetic construct
QQYSNYPWT

SEQ ID NO: 212 - protein, 107 aa, Synthetic construct
DIQMTQSPSSLSASVGDRVTITCKASQNVVSAVAWYQQKPGQAPRRLIYSASNRYTGVPDRFSGSGSGTDFTLTISSLQAEDLADYYCQQYSNYPWTFGGGTKVEIK

SEQ ID NO: 213 - DNA, 33 bp, Synthetic construct; identical to SEQ ID NO: 149
  source 1..33: mol_type = other DNA

SEQ ID NO: 214 - DNA, 21 bp, Synthetic construct
  source 1..21: mol_type = other DNA
agcgcctccaacaggtacacc

SEQ ID NO: 215 - DNA, 27 bp, Synthetic construct
  source 1..27: mol_type = other DNA
caacagtacagcaattacccttggacg

SEQ ID NO: 216 - DNA, 321 bp, Synthetic construct
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggaggctgatctatagcgcctccaacaggtacaccggggtcccagacaggttcagcggcagtggatctgggacagatttcactctcaccatcagcagcctgcaggccgaagatctggcagactattactgtcaacagtacagcaattacccttggacgttcggcggcgggaccaaggtggaaatcaaa

SEQ ID NO: 217 - protein, 23 aa, Synthetic construct
DIQMTQSPSSLSASVGDRVTITC

SEQ ID NO: 218 - protein, 15 aa, Synthetic construct
WYQQKPGQAPRRLIY

SEQ ID NO: 219 - protein, 32 aa, Synthetic construct; identical to SEQ ID NO: 203

SEQ ID NO: 220 - protein, 10 aa, Synthetic construct
FGGGTKVEIK

SEQ ID NO: 221 - DNA, 69 bp, Synthetic construct; identical to SEQ ID NO: 189
  source 1..69: mol_type = other DNA

SEQ ID NO: 222 - DNA, 45 bp, Synthetic construct
  source 1..45: mol_type = other DNA
tggtatcagcagaaaccagggcaggctcctaggaggctgatctat

SEQ ID NO: 223 - DNA, 96 bp, Synthetic construct; identical to SEQ ID NO: 207
  source 1..96: mol_type = other DNA

SEQ ID NO: 224 - DNA, 30 bp, Synthetic construct; identical to SEQ ID NO: 160
  source 1..30: mol_type = other DNA

SEQ ID NO: 225 - protein, 330 aa, Synthetic construct [sequence omitted]

SEQ ID NO: 226 - protein, 330 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 227 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 228 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 229 - protein, 107 aa, Synthetic construct [sequence omitted]

SEQ ID NO: 230 - protein, 449 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 449 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 232 - protein, 446 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 233 - protein, 446 aa, Synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 234 - protein, 214 aa, Synthetic construct
DIQMTQSPSSLSASVGDRVTITCKASQNVVSAVAWYQQKPGQAPRLLIYSASNRYTGVPDRFSGSGSGTDFTLTISSLQAEDLADYYCQQYSNYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 330 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 330 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 237 - protein, 327 aa, Synthetic construct; identical to SEQ ID NO: 228

SEQ ID NO: 238 - protein, 107 aa, Synthetic construct [sequence omitted]

SEQ ID NO: 239 - protein, 445 aa, Synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 240 - protein, 442 aa, Synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 241 - protein, 445 aa, Synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAYWLFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 242 - protein, 445 aa, Synthetic construct; identical to SEQ ID NO: 241

SEQ ID NO: 243 - protein, 214 aa, Synthetic construct
DIQMTQSPSTLSASVGDRVTITCKASQDVGTAVDWYQQKPGQAPKLLIYWASTRHTGVPDRFSGSGSGTDFTLTISRLQPEDFAVYYCQQYNSYPLTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 10 aa, Synthetic construct
GYTFTEYTIH

SEQ ID NO: 245 - protein, 115 aa, Synthetic construct
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 246 - protein, 107 aa, Synthetic construct
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

(19) United States
(12) Patent Application Publication
(10) Pub. No.: US 2024/0374764 A1
(43) Pub. Date: Nov. 14, 2024

(54) COMBINATION RADIOTHERAPY

(71) Applicants: Telix Pharmaceuticals (Innovations) Pty Ltd., Melbourne (AU); Merck Patent GmbH, Darmstadt (DE)

(72) Inventors: Michael Paul Wheatcroft, North Melbourne (AU); Edwin Bingbing Yan, North Melbourne (AU); Andrew Mark Scott, Heidelberg (AU); Cameron Johnstone, North Melbourne (AU); Astrid Zimmermann, Muehltal (DE); Frank Zenke, Darmstadt (DE)

(73) Assignees: Telix Pharmaceuticals (Innovations) Pty Ltd., Melbourne (AU); Merck Patent GmbH, Darmstadt (DE)

(21) Appl. No.: 18/684,604

(22) PCT Filed: Aug. 17, 2022
(86) PCT No.: PCT/AU2022/050911

§ 371 Date: Feb. 16, 2024

(30) Foreign Application Priority Data
Aug. 17, 2021 (AU) 2021902557
Aug. 18, 2021 (AU) 2021902582

(51) Int. Cl. A61K 51/10; A61K 31/5377; A61P 35/04; C07K 16/28; C07K 16/40

(52) U.S. Cl. CPC A61K 51/1075; A61K 31/5377; A61P 35/04; C07K 16/2818; C07K 16/2827; C07K 16/40; A61K 2121/00

(57) ABSTRACT

The present invention relates to methods for treating diseases and conditions characterised by aberrant cell growth, e.g., cancers, comprising administering a combination of a DNA repair inhibitor and a molecular targeted radioimmunotherapeutic agent.

### FIELD OF THE INVENTION

The invention relates to a combined modality for the treatment of cancers, wherein the modality comprises administration of inhibitors of DNA Protein Kinase (DNA-PK) and molecular targeted radiotherapy.

### RELATED APPLICATIONS

This application claims priority from Australian provisional patent applications AU 2021902557 and AU 2021902582, the entire contents of both of which are incorporated herein by reference.

### BACKGROUND OF THE INVENTION

To ensure the accurate maintenance and transfer of genetic information to progeny, mammalian cells have evolved sophisticated mechanisms to sense DNA damage, coordinate its repair, and prevent potential tumorigenic effects; this is collectively known as the DNA damage response (DDR). Defects in the DDR contribute to genomic instability and represent one of the key hallmarks of cancer.

DNA can be damaged by multiple endogenous and exogenous factors. Many established therapeutic modalities, such as radiotherapy and chemotherapy that attack cancer cell DNA are in clinical use but provide limited benefit to patients with cancer. This is due, at least in part, to the competence of tumour cells to deal with DNA damage.

Diverse types of lesions can be generated in DNA, ranging from base modifications to strand breaks, leading to large deletions or genomic rearrangements. Of those, double-strand breaks (DSB) are considered the most harmful and can have lethal consequences for the cells and organism if left unrepaired. DSB repair is accomplished through two major pathways, homologous recombination-guided repair (HR) and nonhomologous end joining (NHEJ). HR requires an intact DNA strand as a template for break repair and is restricted to the S and G2 phases of the cell cycle. Therefore, HR is considered less error prone than NHEJ. Conversely, NHEJ repairs DSBs in the absence of a template and leads to alterations in the repaired DNA. However, NHEJ is functional in all phases of the cell cycle and is believed to participate in the repair of over 80% of DSBs induced by ionizing radiation (IR) in cancer cells.

DNA-dependent protein kinase (DNA-PK) is a serine/threonine kinase and a key driver of NHEJ repair, working in co-ordination with five additional factors, Ku70, Ku80, XRCC4, ligase IV, and Artemis. A heterodimer consisting of Ku70 and Ku80 binds specifically to DSBs, recruits and activates the catalytic subunit DNA-PKc, which in turn recruits the XRCC4/ligase IV heterodimer responsible for resealing the break. Trimming of the DSB ends may require Artemis and other DNA polymerases specialized in repair-mediated DNA polymerization. The activation of DNA-PK through autophosphorylation is essential for proper execution of the repair process

Potentiating radiotherapy and chemotherapy by inhibiting DNA damage repair has been proposed as a therapeutic strategy to improve, for instance, outcomes for patients with solid tumours. However, the success of such combination therapies has often been hampered by toxicity issues.

There remains a need for improved treatments of cancer and other disorders characterised by aberrant cell function and growth. In particular, there is a need for increased efficacy for such treatments, while at the same time, maintaining a favourable toxicity and side effect profile.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect, the invention provides a method for treating a disease or disorder characterised by aberrant cell growth and function in a subject, the method comprising administering to a subject in need thereof, a combination therapy comprising:
i) a DNA-PK inhibitor (DNA-PKi);
ii) a molecular targeted radiotherapeutic capable of cellular internalisation and/or retention in the circulation of the subject;
   wherein the radiotherapeutic comprises a radionuclide that is a beta emitter;
      thereby treating the disease or disorder characterised by aberrant cell growth and function in the subject.

In a second aspect, the invention provides a method for treating a disease or disorder characterised by aberrant cell growth and function in a subject, the method comprising administering to a subject in need thereof, a combination therapy comprising:
i) (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof;
ii) a molecular targeted radiotherapeutic capable of cellular internalisation and/or retention in the circulation of the subject;
   thereby treating the disease or disorder characterised by aberrant cell function in the subject.

In preferred embodiments of any aspect of the invention, the molecular targeted radiotherapeutic is a radioimmunoconjugate. Preferably, the radioimmunoconjugate comprises an antibody or antigen binding fragment thereof for binding to an antigen associated with the disease or disorder for which treatment is required; to which is conjugated a radionuclide.

The antibody may be an antibody (e.g. a monoclonal antibody) which is in itself an immunotherapeutic agent which binds to certain cells or proteins and then stimulates the patient's immune system to attack those cells. In this case, the radiotherapeutic acts in tandem with the immunotherapeutic effects of the antibody. Alternatively, the antibody may act solely as a targeting agent and does not provoke any immunotherapeutic effects by itself. In this case, it is solely the radionuclide conjugated to the antibody acts as the active, cell-destroying agent, supported in the combination therapy methods of the present invention by a DNA-PKi as described herein.

In particularly preferred embodiments of any aspect of the invention, the molecular targeted radiotherapeutic is a monoclonal antibody comprising an antigen binding domain for binding to an antigen associated with the disease or disorder for which treatment is required, wherein the monoclonal antibody is conjugated to a radionuclide for providing a radiotherapeutic dose to cells expressing the antigen.

In any aspect of the invention, the disease or disorder characterised by aberrant cell growth or function is a cancer. However, it will be appreciated that the invention also applies to the treatment of other diseases or conditions where cell replication is unchecked, as further described herein.

It will be appreciated that where the disease or disorder to be treated is a cancer, the molecular targeted radiotherapeutic, preferably a radiolabelled antibody, binds to a tumour-specific or tumour-associated antigen, expressed by the cancer cells to be treated.

In preferred embodiments of any aspect of the invention, the disease or disorder to be treated is a cancer characterised by the expression of carbonic anhydrase IX (CAIX). Accordingly, in such embodiments, the molecular targeted radiotherapeutic preferably comprises an antibody or antigen binding fragment thereof capable of specifically binding to CAIX. Examples of cancers which may be treated accordingly include renal cell carcinoma (including clear renal cell carcinoma), colon cancer, breast cancer, lung cancer, cervical cancer and melanoma.

In preferred embodiments of any aspect of the invention, the disease or disorder to be treated is a cancer characterised by the expression of prostate specific membrane antigen (PSMA). Accordingly, in such embodiments, the molecular targeted radiotherapeutic preferably comprises an antibody or antigen binding fragment thereof capable of specifically binding to PSMA. Examples of cancers which may be treated accordingly include prostate cancer, bladder cancer, testicular-embryonal cancer, neuroendocrine cancer, renal cell carcinoma, and breast cancer.

In accordance with any aspect the invention, radioimmunotherapeutic, preferably a radiolabelled monoclonal antibody, may be labelled with any suitable radionuclide that can be used to provide a therapeutic dose of radiation to a cell. Examples of suitable therapeutic radionuclides include: an alpha emitter selected from the group consisting of Astatine-²¹¹ (²¹¹At), Bismuth-²¹² (²¹²Bi), Bismuth-²¹³ (²¹³Bi), Actinium-²²⁵ (²²⁵Ac), Radium-²²³ (²²³Ra), Lead-²¹² (²¹²Pb), Thorium-²²⁷ (²²⁷Th), and Terbium-¹⁴⁹ (¹⁴⁹Tb). In some embodiments, the radionuclide is ²²⁵Ac. In other embodiments, the radionuclide is ²¹¹Astatine.

In preferred aspects of the invention the radionuclide is a beta or beta/gamma emitter, selected from the group consisting of: Lutetium-¹⁷⁷ (¹⁷⁷Lu), Yttrium-⁹⁰ (⁹⁰Y), Iodine-¹³¹ (¹³¹I), Samarium-¹⁵³ (¹⁵³Sm), Holmium-¹⁶⁶ (¹⁶⁶Ho), Rhenium-¹⁸⁶ (¹⁸⁶Re), or Rhenium-¹⁸⁸ (¹⁸⁸Re). In some embodiments, the radionuclide is ¹⁷⁷Lutetium. In other embodiments, the radionuclide is ¹⁸⁸Rhenium.

In a particularly preferred embodiment of any aspect of the invention, the disease or disorder requiring treatment is a cancer characterised by the expression of CAIX. Accordingly, there is provided a method for treating a cancer characterised by the expression of CAIX, the method comprising administering to a subject in need thereof, a combination therapy comprising:
i) (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof;
ii) an antibody or antigen binding fragment thereof for binding to CAIX, wherein the antibody or fragment thereof is conjugated to a radionuclide for delivering a radiotherapeutic dose to the cancer;
   thereby treating the cancer in the subject. Preferably, the radionuclide is a beta or beta/gamma emitter and the antibody or antigen binding fragment for binding to CAIX is one as described herein, preferably comprising an antigen binding domain comprising an amino acid sequence as defined in any of SEQ ID NOs: 52, 68, 84, 100 and 116 and an amino acid sequence as defined in any of SEQ ID NOs: 132, 148, 164, 180, 196 and 212; most preferably, wherein the antibody comprises the amino acid sequences set out at SEQ ID NO: 231 and 234 herein.

In a particularly preferred embodiment, the disease or disorder requiring treatment is a cancer characterised by the expression of PSMA. Accordingly, the invention also provides a method for treating a cancer characterised by the expression of PSMA, the method comprising administering to a subject in need thereof, a combination therapy comprising:
i) (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof;
ii) an antibody or antigen binding fragment thereof for binding to PSMA, wherein the antibody or fragment thereof is conjugated to a radionuclide for delivering a radiotherapeutic dose to the cancer;
   thereby treating the cancer in the subject. Preferably, the radionuclide is a beta or beta/gamma emitter. Preferably the antibody or antigen binding fragment for binding to PSMA is one as described herein, preferably an antibody having the CDRs of antibody J591, as described herein. In preferred embodiments, the antibody comprises a heavy chain CDR1 as set forth in any of SEQ ID NOs: 1, 17 and 244, a CDR2 as set forth in SEQ ID NO: 2 or 18 and a CDR3 as set forth in SEQ ID NO: 3 or 19; and a light chain CDR1 as set forth in SEQ ID NO: 33, a CDR2 as set forth in SEQ ID NO: 34 and a CDR3 as set forth in SEQ ID NO: 35. In further preferred embodiments, the antibody or antigen binding fragment thereof comprises an antigen binding domain comprising the CDRs of the heavy chain variable domain as defined in any of SEQ ID NOs: 4 or 20; and the CDRs of the light chain variable domain as defined in SEQ ID NO: 36. In certain embodiments, the antibody comprises the amino acid sequences set out at SEQ ID NO: 239 and 243 herein.

In any aspect of the invention, the molecular targeted radiotherapeutic and the DNA-PKi may be administered sequentially in either order, or simultaneously. In a particular embodiment, the radiotherapeutic and the DNA-PKi are administered sequentially in either order. In a further embodiment, the radiotherapeutic may be administered before the DNA-PKi. In this case, the DNA-PKi may be administered at a later time, on the same day as the radiotherapeutic. Preferably, the DNA-PKi is administered no more than 15 days after administration of the radiotherapeutic, such as 1-15 days, preferably 4-10 days, more preferably 2-8 days, most preferably 1-5 days after administration of the radiotherapeutic. In preferred examples, the DNA-PKi is administered one day after the administration of the radiotherapeutic. Administration of DNA-PKi in this context may comprise a single administration of DNA-PKi, or administration of DNA-PKi over one or more days, for instance for a duration as described below.

It will also be appreciated that the DNA-PKi and the radiotherapeutic may be administered via the same or via differing routes of administration. For example, in preferred embodiments, the radiotherapeutic may be administered intravenously, while the DNA-PKi may be administered orally.

In a preferred embodiment of any aspect of the invention, the molecular targeted radiotherapeutic is administered at a dose level below the level required for a monotherapy response. This indicates a synergistic effect between the molecular targeted radiotherapeutic and the DNA-PKi. Preferably, the molecular targeted radiotherapeutic is administered at doses of greater than 10%, preferably greater than 20% less radioactivity compared to the monotherapy response (i.e. the therapy which involves administration of the molecular targeted radiotherapeutic only), preferably 20-50% less radioactivity compared to the monotherapy response. In the alternative or most preferably in addition to said dose of the molecular targeted radiotherapeutic, the DNA-PKi is administered at a dose level below the maximum tolerated dose level, for instance at a dose of up to 90%, 85%, 80%, 75%, 60%, 65%, 60% or 55% of the maximum tolerated dose level, and/or at least 10%, or 20%, 30%, 40% or 50% of the maximum tolerated dose level of the combination.

The DNA-PKi may be administered at a dose of 0.02-100 mg/kg, preferably 0.02-50 mg/kg bodyweight. The daily dose in particular may be between 0.02 and 100 mg/kg of body weight, for instance at least 1 mg/kg body weight, at least 2 mg/kg body weight, at least 3 mg/kg body weight, at least 4 mg/kg body weight or at least 5 mg/kg body weight and up to 25, 30, 40, 45, 50, 60, 70, 80, 90 or 100 mg/kg body weight. The DNA-PKi may, for instance, be administered at a dose of between 1 to 800 mg, such as 50-400 mg, 50 to 500 mg, 5 to 600 mg, more preferably 100 to 400 mg, 100 to 300 mg, 100 to 250 mg, 100 to 200 mg, preferable once daily. Alternatively the DNA-PKi may be administered at a dose of between 150-400 mg by administration twice daily (b.i.d).

The DNA-Pki may be administered at a dose of 0.01 mg to 1 g per dosage unit, preferably between 1 to 700 mg, particularly preferably 5 to 200 mg.

In alternative embodiments, the DNA-PKi may be administered each day over the course of the treatment. In other embodiments, the DNA-PKi may be administered each day starting the day after administration of the radiotherapeutic. For example, the DNA-PKi may be administered every day, for at least 7 days, at least 10 days, at least 14 days, at least 21 days, at least 28 days, or longer.

In particularly preferred embodiments, the DNA-PKi is M3814 and the dosing regimen is within one of the following ranges: 25 to 600 mg, 50 to 600 mg, 100 to 600 mg, 150-600 mg, 175 to 500 mg, 200 to 500 mg, 300 to 400 mg, 50 to 300 mg, 75 to 275 mg, 100 to 250 mg or 100 to 200 mg or a combination thereof. In especially preferred embodiments, the afore-stated dose is administered once daily, but may advantageously also be administered twice daily (b.i.d.). M3814 may, for instance, be administered at a dose of 75 mg, 100 mg, 125 mg, 150 mg, 175 mg, 200 mg, 250 mg, 275 mg or 300 mg, 350 or 400 mg preferably once a day, but suitably also b.i.d, Twice daily administration would most preferably be for doses of 300 mg or more.

One advantage of the present invention, and a distinction from EBRT approaches to delivering radiotherapy, is that the radiotherapeutic is not required to be administered each and every day of the treatment protocol. Accordingly, in any embodiment of the invention, the radiotherapeutic may be administered at intervals of approximately once a week, approximately once every two weeks, approximately once every three weeks; approximately once every four weeks, or at a greater dosing interval. In preferred embodiments, the radiotherapeutic is administered in two doses, at least 7 days, at least 10 days, at least 14 days, at least 21 days, or at least 28 days, at least 35 days, at least 42 days, at least 49 days, at least 56 or more days apart, or in 3 doses, at least 7 days, at least 10 days, at least 14 days, at least 21 days, at least 28 days, at least 35 days, at least 42 days, at least 49 days, at least 56 or more days apart. It will be appreciated that additional dosing of the radiotherapeutic may be required. In certain embodiments, a single administration of the radiotherapeutic may be all that is required for successful treatment and accordingly, the present invention contemplates treatment regimens where the radiotherapeutic is administered once only, along with administration of a DNA-PKi as herein described.

In some embodiments, the treatment comprises one or more treatment cycles, wherein a treatment cycle comprises administration of the radiotherapeutic on the first day of the cycle, followed by administration of DNA-PKi starting on the second, third, fourth, fifth, sixth or seventh day of the cycle, for at least 7 days, for instance 14 days, resulting, for instance, in a 14-day or 15-day cycle. In some embodiments, a treatment cycle comprises administration of the radiotherapeutic on the first day of the cycle, followed by administration of DNA-PKi starting on the second, third, fourth, fifth, sixth or seventh day of the cycle, for at least 20 days, resulting, for instance, in a 21- or 22-day cycle. Starting DNA-PKi treatment on the second day of the cycle is preferred. A treatment may, for instance, include 1, 2, 3 or more of such treatment cycles, optionally with a treatment break.

In embodiments where the treatment comprises more than one treatment cycle, a subsequent treatment cycle may commence immediately after completion of a first treatment cycle (such as the days after completion of the first treatment cycle), or may commence several days or weeks after completion of a first treatment cycle and after a period during which no radiotherapeutic or DNA-PKi is administered (ie a treatment break). The period of treatment break may be at least one day, or may be at least one week, at least two weeks, at least three weeks or longer.

In some embodiments, the treatment may comprise at least two treatment cycles, or at least three treatment cycles, wherein each treatment cycle comprises administration of the radiotherapeutic on the first day of the cycle, followed by administration of the DNA-PKi on subsequent days (eg, commencing on the second, third or fourth days) of the treatment cycle, and comprising administration of the DNA-PKi up to at least the 7^{th}, at least the 14^{th} or at least the 21^{st} days of the treatment cycle; or comprising administration of the DNA-PKi for a period of at least 7 days, at least 14 days or at least 21 days. The period between the end of a first treatment cycle and the commencement of a second treatment cycle (ie, wherein no DNA-PKi and no radiotherapeutic are administered) may be at least 7 days, at least 14 days, at least 21 days, at least 28 days, at least 35 days, at least 42 days, at least 49 days, at least 56 days or more.

For example, a treatment cycle may comprise administration of the radiotherapeutic on the first day of the cycle, followed by administration of DNA-PKi starting on the second day of the cycle, for a period of at least 7 days, at least 14 days or at least 20 days resulting, for instance, in a 21-day cycle. The commencement of a second treatment cycle may be delayed for a period of at least 2 days, at least 5 days, 7 days, at least 14 days, at least 21 days, at least 28 days, at least 35 days, at least 42 days, at least 49 days, at least 56 days or more, resulting in the commencement of the second treatment cycle, for example at least day 56 following initial dosing of the radiotherapeutic. In such embodiments, the second treatment cycle may substantially replicate the first treatment cycle, for example, commencing with administration of the radiotherapeutic on the first day of the second treatment cycle, followed by administration of the DNA-PKi starting on the second day of the second cycle, and administered for at least 7 days, at least 14 days, or at least 21 days, resulting in an overall treatment period, for instance of at least 77 days.

In another example, a treatment cycle may comprise administration of the radiotherapeutic on the first day of the cycle, followed by administration of DNA-PKi starting on day 4 of the cycle, for a period of at least 7 days, at least 14 days, at least 17 days or at least 20 days resulting, for instance, in a 21-day cycle. The commencement of a second treatment cycle may be delayed for a period of at least 2 days, at least 5 days, 7 days, at least 14 days, at least 21 days, at least 28 days, at least 35 days, at least 42 days, at least 49 days, at least 56 days or at least 63 days or more, resulting in the commencement of the second treatment cycle, for example on at least day 85 following initial dosing of the radiotherapeutic (for example, where the initial dosing is on day 1, DNA-PKi treatment is from day 4 to 21, optionally where a treatment break is from day 22 to day 84). In such embodiments, the second treatment cycle may substantially replicate the first treatment cycle, for example, commencing with administration of the radiotherapeutic on the first day of the second treatment cycle, followed by administration of the DNA-PKi starting on day 4 of the second cycle, and administered for at least 7 days, at least 14 days, at least 17 days or at least 20 days, resulting in an overall treatment period, for instance of at least 84 days.

In further embodiments, the treatment may comprise at least three treatment cycles, for instance, where each treatment cycle comprises administration of the radiotherapeutic on the first day of the cycle, followed by administration of the DNA-PKi on subsequent days, for a period of least 7 days, or at least 14 days. A treatment break may be included between the end of first treatment cycle and before the commencement of the second treatment cycles and similarly, between the end of the second treatment cycle and commencement of the third treatment cycles, optionally wherein each treatment break is for a period of at least 2 days, at least 5 days, at least 7 days, at least 10 days, at least 14 days or at least 21 days, at least 28 days, at least 35 days, at least 42 days, at least 49 days, at least 56 days, at least 63 days or more; most preferably wherein the treatment break is for at least 7 days.

Suitable time schedules for treatments or treatment cycles are also described in the examples. Those time schedules are generally also suitable for combinations of other DNA-PKis and radiotherapeutics, and other cancer or tumour types.

The combination therapy of the present invention may be used alone or in combination with other treatment modalities including surgery, external beam radiation therapy, chemotherapy, other radionuclides, or tissue temperature adjustment etc. This forms a further, preferred embodiment of the method of the invention and formulations/medicaments may correspondingly comprise at least one additional therapeutically active agent such as another radioactive agent or a chemotherapeutic agent.

In any aspect of the invention, optionally, the therapy may further comprise iii) an additional anti-cancer therapy selected from the group consisting of: an immune check-point modulator, a chemotherapeutic, a radiation sensitiser, and EBRT.

In any embodiment, the additional anti-cancer therapy may comprise an immune checkpoint modulator. The immune checkpoint modulator may be an immune checkpoint inhibitor selected from: an inhibitor of PD-1, PD-L1 and CTLA-4 or any other immune checkpoint inhibitor described herein.

Optionally, the immune checkpoint inhibitor is an inhibitor of PD-1 selected from: pembrolizumab nivolumab, cemiplimab, spartalizumab, camrelizumab, sintilimab, tislelizuma, toripalimab, dostarlimab, INCMGA00012, AMP-224 and AMP-514.

Optionally, the immune checkpoint inhibitor is an inhibitor of PD-L1 selected from: atezolizumab, avelumab, durvalumab, KN035, CK-301, AUNP12, CA-170, and BMS-986189.

The immune checkpoint inhibitor may be an inhibitor of CTLA-4, selected from: ipilimumab and tremelimumab.

In any embodiment, the additional anti-cancer therapy is a chemotherapeutic, as further described herein.

In any embodiment of the first aspect of the invention, the DNA-PKi may be selected from: M3814, N-methyl-8-[(2S)-1-{[2′-methyl(4′,6′-²H2)-[4,5′-bipyrimidine]-6-yl]amino}propan-2-yl]quinoline-4-carboxamide, 7,9-dihydro-7-methyl-2-[(7-methyl[1,2,4]triazolo[1,5-a]pyridin-6-yl)amino]-9-(tetrahydro-2H-pyran-4-yl)-8H-purin-8-one (AZD7648), 4-ethyl-N-[4-[2-(4-morpholinyl)-4-oxo-4H-1-benzopyran-8-yl]-1-dibenzothienyl]-1-piperazineacetamide (KU-0060648), 2-(4-morpholinyl)-4H-naphtho[1,2-b]pyran-4-one (NU7026), 8-(4-dibenzothienyl)-2-(4-morpholinyl)-4H-1-benzopyran-4-one (NU7441, KU-57788), 3-[4-(4-morpholinyl)pyrido[3′,2′:4,5]furo[3,2-d]pyrimidin-2-yl]-phenol (PI-103), 2-methyl-5-nitro-2-[(6-bromoimidazo[1,2-a]pyridin-3-yl)methylene]-1-methylhydrazide-benzenesulfonic acid, monohydrochloride (PIK-75 HCl), 1-cyclopentyl-3-(1 H-pyrrolo[2,3-b]pyridin-5-yl)-1H-pyrazolo[3,4-d]pyrimidin-4-amine (PP121), SF2523, and analogs thereof.

Preferably, the DNA-PKi is (S)-[2-chloro-4-fluoro-5-(7-morpholin-4-yl-quinazolin-4-yl)-phenyl]-(6-methoxypyridazin-3-yl)-methanol (M3814), or a pharmaceutically acceptable salt thereof.

In accordance with any aspect of the invention, there is a provided (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof, for use in a method of treatment as described herein.

In accordance with any aspect of the invention, there is a provided:
i) an antibody or antigen binding fragment thereof for binding to CAIX, preferably one as described herein, wherein the antibody is conjugated to a radionuclide; or
ii) an antibody or antigen binding fragment thereof for binding to PSMA, preferably one as described herein, wherein the antibody is conjugated to a radionuclide
   for use in a method of treatment as described herein.

Further still, in accordance with the second aspect of the invention, there is provided (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof; for use in a method of treatment as described herein, when administered in a treatment in combination with an antibody or antigen binding fragment thereof for binding to CAIX, preferably one as described herein, wherein the antibody is conjugated to a radionuclide.

Further still, in accordance with the second aspect of the invention, there is provided (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof; for use in a method of treatment as described herein, when administered in a treatment in combination with an antibody or antigen binding fragment thereof for binding to PSMA, preferably one as described herein, wherein the antibody is conjugated to a radionuclide.

In accordance with any aspect of the invention, there is a provided use of:
i) an antibody or antigen binding fragment thereof for binding to CAIX, preferably one as described herein, wherein the antibody is conjugated to a radionuclide; or
ii) an antibody or antigen binding fragment thereof for binding to PSMA, preferably one as described herein, wherein the antibody is conjugated to a radionuclide
   in the manufacture of a medicament, for use in accordance with a method of treatment as described herein.

In accordance with the second aspect of the invention, there is also provided a use of (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof; in the manufacture of a medicament or kit of parts, for use in the treatment of a disease or condition as described herein, wherein the treatment comprises administration of an antibody or antigen binding fragment thereof for binding to CAIX, preferably one as described herein, wherein the antibody is conjugated to a radionuclide.

Further still, in accordance with the second aspect of the invention, there is provided a use of (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof; in the manufacture of a medicament or kit of parts, for use in the treatment of a disease or condition as described herein, wherein the treatment comprises administration of an antibody or antigen binding fragment thereof for binding to PSMA, preferably one as described herein, wherein the antibody is conjugated to a radionuclide.

The present invention also provides a use of:
i) an antibody or antigen binding fragment thereof for binding to CAIX, preferably one as described herein, wherein the antibody is conjugated to a radionuclide; or
ii) an antibody or antigen binding fragment thereof for binding to PSMA, preferably one as described herein, wherein the antibody is conjugated to a radionuclide
   in the manufacture of a first medicament,
   and the use of a DNA-PKi, preferably (S)-[2-chloro-4-fluoro-5-(7-morpholin-4-yl-quinazolin-4-yl)-phenyl]-(6-methoxypyridazin-3-yl)-methanol (M3814), in the manufacture of a second medicament,
   wherein the first and second medicaments are administered in accordance with any method of treatment as described herein.

The present invention also provides a kit for use in any method of the invention described herein. Preferably the kit comprises an antibody as described herein, with a DNA-PKi, preferably (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof; optionally with instructions for use in accordance with the methods of the invention.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: ¹⁷⁷Lu-anti-CAIX antibody SPECT imaging in metastatic renal cell carcinoma SK-RC-52 xenograft model.

FIG. 2: Combination treatment with ¹⁷⁷Lu-anti-CAIX antibody+M3814. Mean tumour volume (mm³) is shown over >100 days following 14 days of treatment. Percentage change in tumour volume is also shown. Kaplan Meier survival curve for 6 MBq dosing shown at 100 days of the study.

FIG. 3: ¹⁷⁷Lu-anti-PSMA antibody SPECT imaging in LNCaP (PSMA^{high}) xenograft model.

FIG. 4: Combination treatment with ¹⁷⁷Lu-anti-PSMA antibody+M3814. Mean tumour volume (mm³) is shown over >100 days following 14 days of treatment. Percentage change in tumour volume is also shown. Kaplan Meier survival curve to day 120.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present invention relates to a new treatment modality, that comprises the combination of an inhibitor of DNA-PK, and a molecular targeted radiotherapeutic capable of cellular internalisation and/or retention in the circulation of the subject.

Conventional treatment for cancer using a DNA-damage repair inhibitor (DDRi) has included concomitant administration of DDRi with administration of external beam radiation (EBRT). Such treatment modalities, while effective, result in increased toxicity of the DDRi administered. Moreover, the treatment method is complex, requiring frequent outpatient visits to receive EBRT. For example, a course of EBRT typically involves several daily treatments (fractions) over days to several weeks, during which the patient receives radiation treatment. Although the radiation beams are targeted to specific regions of the body comprising tumours, the radiation is non-specific, and healthy tissues are also subjected to the radiation during the course of treatment.

The inventors have recognised the benefits of substituting EBRT with molecularly targeted radiotherapy, but in particular, where the radiotherapeutic is capable of internalisation and retention in the circulation of the patient, such as is the case with large molecule (e.g., antibody) radiotherapeutic.

The inventors have demonstrated that successful combination radiotherapy and DNA-PKi treatment can be accomplished using as little as a single administration of a radioimmunoconjugate. This provides significant advantages in the clinical setting, such that the patient requires only a single dose of radiotherapy, administered via injection, rather than frequent visits over the course of an extended period of time to receive EBRT. In addition, the use of molecular targeted radiation delivers the radiotherapeutic dose directly to the tissue requiring treatment, rather than requiring an external beam of radiation to pass through healthy tissues. Molecular targeted radiation therefore reduces unnecessary exposure of healthy tissue to radiation.

Further still, the use of a large molecule radioimmunoconjugate provides a significant advantage compared to the use of a small molecule or peptide, which may bind to the same molecular target and be used to deliver a radiotherapeutic dose. Without wishing to be bound by theory, the inventors believe that the rapid urinary excretion of radiolabelled small molecules or peptides compared to the slower, liver-based degradation of larger molecules such as antibodies, means that smaller doses of radiation can be administered to patients. This has advantages for reducing risk of nephrotoxicity which may otherwise arise through the use of radioconjugates that are excreted via the kidneys.

Moreover, radioimmunoconjugates deliver the radiotherapy in a significantly more targeted manner compared to EBRT and can be designed to be functionally specific for tumour-expressed antigens. For example, small molecules and peptides used to target the same antigens (e.g. PSMA), typically also target non-cancerous tissues expressing the same antigens (in the case of PSMA, lacrimal/salivary glands, ganglia, and small bowel), producing unpleasant side effects. The use of an antibody-based approach for delivery of radiotherapy therefore significantly reduces unwanted side-effects resulting from on-target, off-tumour binding.

Further still, antibodies, and fragments thereof, can be modified to either increase or reduce persistence in the circulation (for example, by altering binding sites for FcRn and reducing serum half-life).

Thus, the inventors believe that the particular combination of antibody-delivered radiotherapy with a DNA-PKi provides for significant advantages in terms of reduced radiation dosing and improved clinical set-up for delivery of the radiotherapy, requiring in some instances, only a single administration of molecular targeted radiation.

The inventors also believe that the use of DNA-PKi in combination with a radioimmunotherapeutic conjugated to a long range radioisotope, such as a beta-emitting radionuclide, provides a further advantage, for example, over the use of shorter range particles, such as alpha-emitting radionuclides. Without wishing to be bound by theory, it is believed that the cross-fire effect that results from beta-emitters can produce improved therapeutic outcomes, particularly in patients with large tumour masses.

### General and Definitions

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more: reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in diagnostic technology, radioimaging, cell culture, molecular genetics, immunology, immunohistochemistry, protein chemistry, and biochemistry).

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

As used herein, the term “antigen binding domain” and shall be taken to mean a region of an antibody that is capable of specifically binding to an antigen, i.e., a V_{H} or a V_{L} or an F_{V} comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens by virtue of an antigen binding domain contained within a F_{V}. This term includes four chain antibodies (e.g., two light chains and two heavy chains), recombinant or modified antibodies (e.g., chimeric antibodies, humanised antibodies, human antibodies, CDR-grafted antibodies, primatised antibodies, de-immunised antibodies, synhumanised antibodies, half-antibodies, bispecific antibodies).

An antibody generally comprises constant domains, which can be arranged into a constant region or constant fragment or fragment crystallisable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are ˜110 amino acids in length) and one or more constant domains at the C-terminus. The constant domain of the light chain (C_{L} which is ˜110 amino acids in length) is aligned with and disulfide bonded to the first constant domain of the heavy chain (C_{H1} which is 330 to 440 amino acids in length). The light chain variable region is aligned with the variable region of the heavy chain. The antibody heavy chain can comprise 2 or more additional C_{H} domains (such as, C_{H2}, C_{H3} and the like) and can comprise a hinge region between the C_{H1} and C_{H2} constant domains. Antibodies can be of any type (e.g., IgG, IgE, IgM, IgD, IgA, and IgY), class (e.g., IgG₁, IgG₂, IgG₃, IgG₄, IgA₁ and IgA₂) or subclass. In one example, the antibody is a murine (mouse or rat) antibody or a primate (such as, human) antibody. In one example the antibody heavy chain is missing a C-terminal lysine residue. In one example, the antibody is humanised, synhumanised, chimeric, CDR-grafted or deimmunised.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an F_{C} region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR₁, CDR₂, and CDR₃, and framework regions (FRs). For example, the variable region comprises three or four FRs (e.g., FR₁, FR₂, FR₃ and optionally FR₄) together with three CDRs. V_{H} refers to the variable region of the heavy chain. V_{L} refers to the variable region of the light chain.

As used herein, the term “complementarity determining regions” (syn. CDRs; i.e., CDR₁, CDR₂, and CDR₃) refers to the amino acid residues of an antibody variable region the presence of which are major contributors to specific antigen binding. Each variable region domain (V_{H} or V_{L}) typically has three CDRs identified as CDR₁, CDR₂ and CDR₃. The CDRs of V_{H} are also referred to herein as CDR H₁, CDR H₂ and CDR H₃, respectively, wherein CDR H₁ corresponds to CDR 1 of V_{H}, CDR H₂ corresponds to CDR 2 of V_{H} and CDR H₃ corresponds to CDR 3 of V_{H}. Likewise, the CDRs of V_{L} are referred to herein as CDR L₁, CDR L₂ and CDR L₃, respectively, wherein CDR L₁ corresponds to CDR 1 of V_{L}, CDR L₂ corresponds to CDR 2 of V_{L} and CDR L₃ corresponds to CDR 3 of V_{L}. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997.

In one example, the CDRs are defined according to the Kabat numbering system. Optionally, heavy chain CDR₂ according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR₂ are not generally involved in antigen binding.

“Framework regions” (FRs) are those variable region residues other than the CDR residues. The FRs of V_{H} are also referred to herein as FR H₁, FR H₂, FR H₃ and FR H₄, respectively, wherein FR H₁ corresponds to FR 1 of V_{H}, FR H₂ corresponds to FR 2 of V_{H}, FR H₃ corresponds to FR 3 of V_{H} and FR H₄ corresponds to FR 4 of V_{H}. Likewise, the FRs of V_{L} are referred to herein as FR L₁, FR L₂, FR L₃ and FR L₄, respectively, wherein FR L₁ corresponds to FR 1 of V_{L}, FR L₂ corresponds to FR 2 of V_{L}, FR L₃ corresponds to FR 3 of V_{L} and FR L₄ corresponds to FR 4 of V_{L}.

As used herein, the term “Fv” shall be taken to mean any protein, whether comprised of multiple polypeptides or a single polypeptide, in which a V_{L} and a V_{H} associate and form a complex having an antigen binding domain, i.e., capable of specifically binding to an antigen. The V_{H} and the V_{L} that form the antigen binding domain can be in a single polypeptide chain or in different polypeptide chains. Furthermore, an Fv of the invention (as well as any protein of the invention) may have multiple antigen binding domains that may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH₂ or CH₃ domain, e.g., a minibody.

A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)₂ fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH₃ domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells. Generally, but not necessarily, reference to binding means specific binding, and each term shall be understood to provide explicit support for the other term.

As used herein, the term “does not detectably bind” shall be understood to mean that an antigen binding protein, e.g. an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding protein is immobilised and contacted with an antigen.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using biosensor analysis (e.g. Biacore or Blitz) in which the antigen binding protein is immobilised and contacted with an antigen.

As used herein, the term “epitope” (syn. “antigenic determinant”) shall be understood to mean a region of the antigen to which an antigen binding protein comprising an antigen binding domain of an antibody binds. Unless otherwise defined, this term is not necessarily limited to the specific residues or structure to which the antigen binding protein makes contact. For example, this term includes the region spanning amino acids contacted by the antigen binding protein and 5-10 (or more) or 2-5 or 1-3 amino acids outside of this region. In some examples, the epitope comprises a series of discontinuous amino acids that are positioned close to one another when antigen binding protein is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

As used herein, the terms “preventing”, “prevent” or “prevention” include administering an antigen binding protein of the invention to thereby stop or hinder the development of at least one symptom of a condition. This term also encompasses treatment of a subject in remission to prevent or hinder relapse.

As used herein, the terms “treating”, “treat” or “treatment” include administering an antigen binding protein described herein to thereby reduce or eliminate at least one symptom of a specified disease or condition.

As used herein, the term “subject” shall be taken to mean any animal including humans, for example a mammal. Exemplary subjects include but are not limited to humans and non-human primates. For example, the subject is a human.

### Inhibitors of Cellular and DNA Damage Repair

In accordance with the methods of the present invention, there is provided a combination therapy comprising a DNA-PK inhibitor (DNA-PKi) and a radioimmunoconjugate. It has been found that this type of combination therapy results in unexpected improvement in the treatment of cancer.

DNA-dependent protein kinase (DNA-PK) is a serine/threonine protein kinase which is activated in conjunction with DNA. Biochemical and genetic data show that DNA-PK consists (a) of a catalytic sub-unit, which is called DNA-PKcs, and (b) two regulatory components (Ku70 and Ku80). In functional terms, DNA-PK is a crucial constituent on the one hand of the repair of DNA double-strand breaks (DSBs) and on the other hand of somatic or V(D)J recombination. In addition, DNA-PK and its components are connected with a multiplicity of further physiological processes, including modulation of the chromatin structure and telomeric maintenance (Smith & Jackson (1999) Genes and Dev 13: 916; Goytisolo et al. (2001) Mol. Cell. Biol. 21: 3642; Williams et al. (2009) Cancer Res. 69: 2100).

The present invention relates to the use of compounds which can inhibit activity of DNA-PK. As used herein, the term “inhibition” or “inhibit” relates to any reduction in the activity which is based on the action of the specific compounds described herein, in that the latter are capable of interacting with the target molecule in such a way that recognition, binding and blocking is made possible. The compounds are distinguished by high affinity to at least one serine/threonine protein kinases, ensuring reliable binding and preferably complete blocking of the kinase activity. The compounds are particularly preferably monospecific in order to guarantee exclusive and direct recognition of the selected kinase. The term “recognition” relates here to any type of interaction between the compound and the said target molecules, in particular covalent or non-covalent bonds, such as, for example, a covalent bond, hydrophobic/hydrophilic interactions, van der Waals forces, ion attraction, hydrogen bonds, ligand/receptor interactions, base pairs of nucleotides or interactions between epitope and antibody binding site.

As used herein, a DNA-PKi is to be understood to include a molecule that substantially inhibits DNA-PK. Preferably, the DNA-PKi inhibits DNA-PK with an IC₅₀ (half maximal inhibitory concentration) of less than 500 nM, preferably less than 250, 100, 50, 10 or 1 nM, at an ATP concentration close to Km (10 μM). The IC₅₀ value may, for instance, be determined by the biochemical assay described further below.

Preferably, the DNA-PKi has a selectivity (ie, specificity) for DNA-PK that is at least 5-fold, at least 10-fold, at least 15-fold or at least 20-fold greater than for other, in particular, related kinases, such as kinases from the PI3K family. Most preferably, the DNA-PKi has a selectivity (specificity) for DNA-PK that is at least 10-fold greater than the specificity for a related serine/threonine, tyrosine or lipid kinase. In preferred embodiments, the DNA-PKi does not substantially inhibit ATR, ATM and mTOR. In preferred embodiments, the selectivity of the DNA-PKi over PI3Kalpha, PI3Kbeta, PI3Kgamma, PI3Kdelta, mTOR, ATM and/or ATR is at least 10-fold, more preferably at least 50-fold or 100-fold. The selectivity may be calculated on the basis of the biochemical IC₅₀ values (e.g. ratio of IC₅₀ PI3Kalpha/IC₅₀DNA-PK).

The ability of a molecule to inhibit DNA-PK can be determined by known methods in the prior art. For example, such methods are described in WO 2014/183850, incorporated herein by reference. Briefly, inhibition can be determined via changes in levels of kinase activity. Measurement of kinase activity is a technique which is well known to the person skilled in the art. Generic test systems for the determination of the kinase activity using substrates, for example histone (Alessi et al. (1996) FEBS Lett. 399(3): 333) or the basic myelin protein, are described in the literature (Campos-Gonzalez & Glenney (1992) JBC 267: 14535). Various assay systems are available for the identification of kinase inhibitors. In the scintillation proximity assay (Sorg et al. (2002) J Biomolecular Screening 7: 11) and the flash-plate assay, the radioactive phos¬phorylation of a protein or peptide as substrate are measured using ATP. In the presence of an inhibitory compound, a decreased radioactive signal, or none at all, is detectable. Furthermore, homogeneous time-resolved fluorescence resonance energy transfer (HTR-FRET) and fluorescence polarisation (FP) technologies are useful as assay methods (Sills et al. (2002) J Biomolecular Screening 191). Other non-radioactive ELISA methods use specific phospho-antibodies (phospho-ABs). The phospho-AB binds only the phosphorylated substrate. This binding can be detected by chemiluminescence using a second peroxidase-conjugated anti-sheep antibody.

The susceptibility of a particular cell to treatment with the compounds according to the invention can be determined by testing in vitro. Typically, a culture of the cell is incubated with a compound according to the invention at various concentrations for a period of time which is sufficient to enable the active agents to induce cell death or to inhibit cell proliferation, cell vitality or migration, usually between about one hour and up to 9 days. For testing in vitro, cultivated cells from a biopsy sample can be used. The amount of cells remaining after the treatment is then determined. The use in vitro takes place, in particular, on samples of mammal species which are suffering from cancer, tumours or metastases. The host or patient can belong to any mammal species, for example a primate species, in particular humans, but also rodents (including mice, rats and hamsters), rabbits, horses, cows, dogs, cats, etc. Animal models are of interest for experimental investigations, providing a model for the treatment of a human disease.

In more detail, biochemical methods for assessing DNA-PK activity may be as described in Kashishian et al. (2003) Molecular Cancer Therapeutics 1257. Briefly, this assay can be carried out in streptavidin-coated 348-well microtitre flashplates. To this end, 1.5 μg of DNA-PK/protein complex and 100 ng of biotinylated substrate, such as, for example, PESQEAFADLWKK-biotin-NH2 (“biotin-DNA-PK peptide”), may be incubated for 90 min at room temperature in a total volume of 36.5 μl (34.25 mM HEPES/KOH; 7.85 mM Tris HCl; 68.5 mM KCl; 5 μM ATP; 6.85 mM MgCl2; 0.5 mM EDTA; 0.14 mM EGTA; 0.69 mM DTT; pH 7.4) with 500 ng of DNA from calf thymus, 0.1 μCi of 33P-ATP and 1.8% of DMSO per well with and without the test compound. The reaction is then stopped using 50 μl/well of 200 mM EDTA. After incubation for a further 30 min at room temperature, the liquid is removed. Each well is washed three times with 100 μl of 0.9% saline solution. A non-specific reaction (blank value) is determined using 10 μM of an innate kinase inhibitor. The radioactivity measurement can be carried out using a TopCount. IC50 values were calculated in RS1.

Cell-based assays for assessing DNA-PK activity may also be used. In one example, HCT116 cells are cultivated at 37° C. and 10% CO₂ in MEM alpha medium with 10% of foetal calf serum and 2 mM glutamine. The cells are detached from the base of the culture vessels with the aid of trypsin/EDTA, centrifuged off in centrifuge tubes, taken up in fresh medium, and the cell density determined. 100,000 cells are sown in 1 ml of culture medium per cavity of a 24-well cell culture plate and cultivated overnight. Next day, 10 μM bleomycin (DNA intercalator and inductor of DNA double-strand breaks) and test substances in fresh culture medium are added to the cells, and these are cultivated for a further six hours. Cell lysis is subsequently carried out, and the cell lysates are added to a blocked 96-well ELISA plate coated with DNA-PK-specific antibodies (Sigma-Aldrich WH0005591M2: total DNA-PK; Abcam ab18192 or Epitomics EM09912: phospho-serine 2056 DNA-PK) and incubated at 4° C. overnight. The 96-well ELISA plates are subsequently treated with a detection antibody (Abcam ab79444: total DNA-PK) and a streptavidin-HRP conjugate. The development of the enzymatic reaction is carried out with the aid of a chemiluminescent reagent, the chemiluminescence may be measured with the aid of a Mithras LB940. The signals with the phospho-DNA-PK-specific antibody are standardised to the signal with the antibody against the total protein DNA-PKc. The determination of IC₅₀ values or of percentage values was carried out by referencing to the signal level of the bleomycin-treated vehicle control group (100% of the control). A DMSO control may be used as blank.

Non-limiting examples of DNA-PK inhibitors include (S)-[2-chloro-4-fluoro-5-(7-morpholin-4-yl-quinazolin-4-yl)-phenyl]-(6-methoxypyridazin-3-yl)-methanol (M3814), N-methyl-8-[(2S)-1-{[2′-methyl(4′,6′-²H2)-[4,5′-bipyrimidine]-6-yl]amino}propan-2-yl]quinoline-4-carboxamide, 7,9-dihydro-7-methyl-2-[(7-methyl[1,2,4]triazolo[1,5-a]pyridin-6-yl)amino]-9-(tetrahydro-2H-pyran-4-yl)-8H-purin-8-one (AZD7648), 4-ethyl-N-[4-[2-(4-morpholinyl)-4-oxo-4H-1-benzopyran-8-yl]-1-dibenzothienyl]-1-piperazineacetamide (KU-0060648), 2-(4-morpholinyl)-4H-naphtho[1,2-b]pyran-4-one (NU7026), 8-(4-dibenzothienyl)-2-(4-morpholinyl)-4H-1-benzopyran-4-one (NU7441, KU-57788), 3-[4-(4-morpholinyl)pyrido[3′,2′:4,5]furo[3,2-d]pyrimidin-2-yl]-phenol (PI-103), 2-methyl-5-nitro-2-[(6-bromoimidazo[1,2-a]pyridin-3-yl)methylene]-1-methylhydrazide-benzenesulfonic acid, monohydrochloride (PIK-75 HCl), 1-cyclopentyl-3-(1H-pyrrolo[2,3-b]pyridin-5-yl)-1H-pyrazolo[3,4-d]pyrimidin-4-amine (PP121), SF2523 (CAS N° 1174428-47-7), and analogs thereof. Preferably, the DNA-PKi is (S)-[2-chloro-4-fluoro-5-(7-morpholin-4-yl-quinazolin-4-yl)-phenyl]-(6-methoxypyridazin-3-yl)-methanol (M3814), or a pharmaceutically acceptable salt thereof.

In accordance with the second aspect of the invention, and a preferred embodiment of the first aspect of the invention, the DNA-PKi is (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof.

As used herein (S)-[2-chloro-4-fluoro-5-(7-morpholin-4-yl-quinazolin-4-yl)-phenyl]-(6-methoxypyridazin-3-yl)-methanol is a DNA-PK inhibitor, and is also known by the names M3814 and peposertib. M3814 is described in detail in United States patent application US 2016/0083401, the entirety of which is hereby incorporated herein by reference. M3814 is designated as compound 136 in Table 4 of US 2016/0083401. M3814 is active in a variety of assays and therapeutic models demonstrating inhibition of DNA-PK. M3814 is an orally bioavailable, potent and selective ATP-competitive inhibitor of DNA-PK, as demonstrated by crystallographic and enzyme kinetics studies. DNA-PK, together with five additional protein factors (Ku70, Ku80, XRCC4, Ligase IV and Artemis) plays a critical role in the repair of DSB via NHEJ. Kinase activity of DNA-PK is essential for proper and timely DNA repair and the long-term survival of cancer cells. Without wishing to be bound by any particular theory, it is believed that the primary effects of M3814 are suppression of DNA-PK activity and DNA double strand break (DSB) repair, leading to altered repair of DNA and potentiation of antitumor activity of DNA-damaging agents.

### Radiotherapeutic

Radiolabelled targeting moieties (also known as radioimmunoconjugates) are designed to target a protein or receptor that is upregulated in a disease state and/or specific to diseased cells (e.g., tumor cells) to deliver a radioactive payload to damage and kill cells of interest. “Radioimmunotherapy” refers to this therapy when the targeting moiety comprises an antibody, typically a monoclonal antibody.

Radioactive decay of the payload produces an alpha, beta, or gamma particle or Auger electron that can cause direct effects to DNA (such as single or double stranded DNA breaks) or indirect effects such as by-stander or crossfire effects.

Radioimmunoconjugates typically contain a biological targeting moiety (e.g., an antibody or antigen binding fragment thereof that specifically binds to a molecule expressed on or by a tumor, e.g., CAIX or PSMA), a chelating moiety or a metal complex of a chelating moiety (e.g., comprising a radioisotope), and a linker. Conjugates may be formed by appending a bifunctional chelate to the biological targeting molecule so that structural alterations are minimal while maintaining target affinity. A radioimmunoconjugate may be formed by radiolabelling such a conjugate.

Bifunctional chelates structurally contain a chelate, a linker, and a cross-linking group. When developing new bifunctional chelates, most efforts focus around the chelating portion of the molecule. Several examples of bifunctional chelates have been described with various cyclic and acyclic structures conjugated to a targeted moiety.

The term “radioconjugate,” as used herein, refers to any conjugate that includes a radioisotope or radionuclide, such as any of the radioisotopes or radionuclides described herein.

The term “radioimmunoconjugate,” as used herein, refers to any immunoconjugate that includes a radioisotope or radionuclide, such as any of the radioisotopes or radionuclides described herein. The term “immunoconjugate,” as used herein, refers to a conjugate that includes a targeting moiety, such as an antibody, or antigen binding fragment thereof. In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 4, 5, or 8 conjugates per targeting moiety).

The term “radioimmunotherapy,” as used herein, refers a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell, wherein administration of the radioimmunoconjugate kills the cell. Where radioimmunotherapy involves the selective killing of a cell, in some embodiments the cell is a cancer cell in a subject having cancer.

As used herein, the term “radionuclide,” refers to an atom capable of undergoing radioactive decay (e.g., ³H, ¹⁴C, ¹⁵N, ¹⁸F, ³⁵S, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ⁹⁹mTc ¹⁰⁵Rh, ¹⁰⁹Pd, ¹¹¹In, ¹²³I, ¹²⁴I, ¹²⁵I, ¹³¹I, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb ²¹²Bi, ²¹³Bi ²²³Ra, ²²⁵Ac, ²²⁷Th, ²²⁹Th, ⁶⁶Ga, ⁶⁷Ga, ⁶⁸Ga, ⁸²Rb, ¹¹⁷mSn, 201Tl).

The terms radioactive nuclide, radioisotope, or radioactive isotope may also be used to describe a radionuclide. Radionuclides may be used as detection agents, as described above. In some embodiments, the radionuclide is an alpha-emitting radionuclide.

### Antibodies

As used herein, “antibody” refers to a polypeptide whose amino acid sequence including immunoglobulins and fragments thereof which specifically bind to a designated antigen, or fragments thereof. Antibodies may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acid sequences found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains and may include sequence elements found in the same polypeptide chain or in different polypeptide chains. Antibodies typically comprise two identical light polypeptide chains and two identical heavy polypeptide chains linked together by disulfide bonds. The first domain located at the amino terminus of each chain is variable in amino acid sequence, providing the antibody-binding specificities of each individual antibody. These are known as variable heavy (VH) and variable light (VL) regions. The other domains of each chain are relatively invariant in amino acid sequence and are known as constant heavy (CH) and constant light (CL) regions. Light chains typically comprise one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Methods for generating antibodies are known in the art and/or described in Harlow and Lane (editors) Antibodies: A Laboratory Manual, Cold Spring Harbor Laboratory, (1988). Generally, in such methods dysfunctional P2X₇ receptor or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunised animal at various points following immunisation. One or more further immunisations may be given, if required to achieve a desired antibody titre. The process of boosting and titreing is repeated until a suitable titre is achieved. When a desired level of immunogenicity is obtained, the immunised animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunised with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunisation, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunised animal are then fused with cells of an immortal myeloma cell, generally derived from the same species as the animal that was immunised with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titre, such as, for example, by flow cytometry and/or immunohistochemstry and/or immunoassay (e.g. radioimmunoassay, enzyme immunoassay, cytotoxicity assay, plaque assay, dot immunoassay, and the like).

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody for use according to the methods of the present invention may be a synthetic antibody. For example, the antibody is a chimeric antibody, a humanised antibody, a human antibody synhumanised antibody, primatised antibody or a de-immunised antibody

Antibodies described herein can include, for example, monoclonal antibodies, polyclonal antibodies, multispecific antibodies, human antibodies, humanized antibodies, camelid antibodies, chimeric antibodies, single-chain Fvs (scFv), disulfide-linked Fvs (sdFV), and anti-idiotypic (anti-Id) antibodies, and antigen-binding fragments of any of the above. In some embodiments, the antibody or antigen-binding fragment thereof is humanized. In some embodiments, the antibody or antigen-binding fragment thereof is chimeric. Antibodies can be of any type (e.g., IgG, IgE, IgM, IgD, IgA and IgY), class (e.g., IgG1, IgG2, IgG3, IgG4, IgA1 and IgA2) or subclass.

Preferably, the antibody is of a format and size that ensures that the antibody is not substantially subject to renal clearance, but rather, is predominantly subject to hepatic clearance.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)2 fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

Antibodies or fragments described herein can be produced by any method known in the art for the synthesis of antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using the methods described in, e.g., Morrison, 1985, Science 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods 248:1-6 (2001); and Tutt et al., J. Immunol. 147: 60 (1991).

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG₁, IgG₂, IgG₃ and IgG₄. In one example, the constant region is human isotype IgG₄ or a stabilised IgG₄ constant region.

In one example, the Fc region of the constant region has a reduced ability to induce effector function, e.g., compared to a native or wild-type human IgG₁ or IgG₃ Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG₄ Fc region (i.e., from an IgG₄ constant region), e.g., a human IgG₄ Fc region. Sequences of suitable IgG₄ Fc regions will be apparent to the skilled person and/or available in publically available databases (e.g., available from National Center for Biotechnology Information).

In one example, the constant region is a stabilised IgG₄ constant region. The term “stabilised IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG₄ antibody dissociates to form two molecules each containing a single heavy chain and a single light chain.

In one example, a stabilised IgG₄ constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG₄, this residue is generally a serine. Following substitution of the serine for proline, the IgG₄ hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues that are involved in inter-heavy chain disulfide bonds. It is generally defined as stretching from Glu226 to Pro243 of human IgG₁ according to the numbering system of Kabat. Hinge regions of other IgG isotypes may be aligned with the IgG₁ sequence by placing the first and last cysteine residues forming inter-heavy chain disulphide (S-S) bonds in the same positions (see for example WO2010/080538).

Additional examples of stabilised IgG₄ antibodies are antibodies in which arginine at position 409 in a heavy chain constant region of human IgG₄ (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG₁ Fc region comprising a substitution at one or more positions selected from the group consisting of 268, 309, 330 and 331. In another example, the Fc region is an IgG₁ Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one CH₂ domain from an IgG₄ antibody and at least one CH₃ domain from an IgG₁ antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present invention also contemplates additional modifications to an antibody or antigen binding protein comprising an Fc region or constant region.

For example, the antibody comprises one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

### Antibodies for Binding to CAIX

As used herein, CAIX refers to the transmembrane protein carbonic anhydrase IX (CAIX), which is a member of the large family of carbonic anhydrase enzymes which share the ability to catalyse the reversible hydration of carbon dioxide to carbonic acid, leading to a decrease in pH. Up-regulation of CAIX gene expression occurs in response to hypoxia via direct transcriptional activation by hypoxia inducible factor-1 alpha (HIF-1a), and is believed to be involved in sensing and maintaining the acidic environment of hypoxic cells, particularly within the hypoxic regions of tumours.

The term “carbonic anhydrase IX” and “CAIX”, “CA9”, “MN” and “G250” may be used interchangeably.

CAIX is highly expressed in different tumour types and has relatively low expression in normal tissues; has an important role in tumour progression, acidification and metastasis; and is located on the extracellular surface of cell membranes, allowing for efficient targeting by antibodies or small molecule inhibitors.

Various molecules for binding to CAIX are known, including radiolabelled small molecules, antibodies and antibody fragments for use in immunohistochemistry or immuno-imaging techniques.

Anti-CAIX antibodies, variants and fragments thereof are described, for example, in EP 637 336, WO 93/18152, WO 95/34650, WO 00/24913, WO 02/063010, WO 04/025302, WO 05/037083, WO 2011/139375, WO 2014/096163, WO 2019/122025 and foreign counterparts thereof. Further, WO 02/062972 describes a hybridoma cell line DSM ACC 2526 which produces the monoclonal antibody G250. The monoclonal antibody G250 recognizes an antigen preferably expressed on membranes of renal cell carcinoma cells (RCC), but not expressed in normal proximal tubular epithelium.

In another preferred embodiment, the antibody and/or the antibody fragment thereof is selected from the group consisting of polyclonal antibodies, monoclonal antibodies, antigen-binding fragments thereof such as F(ab′)2, Fab′, sFv, dsFv and chimerized, humanized and fully human variants thereof. According to a further preferred embodiment, this anti-CAIX antibody or epitope-binding fragment thereof binds to the amino acid sequence LSTAFARV and/or ALGPGREYRAL.

According to a further particularly preferred embodiment, the CAIX targeting compound is the antibody cG250 and/or an epitope-binding fragment thereof (e.g., as described in EP-B-0 637 336). Preferably the CAIX targeting molecule is chimeric or humanised G250 antibody and/or a fragment thereof. The antibodies for use in the present invention may be produced by any suitable method known in the art including but not limited by methods as described in PCT/EP02/01282 and PCT/EP02/01283, which are incorporated herein by reference.

An especially preferred antibody is cG250, preferably girentuximab (INN), also referred to herein as GmAb. Another especially preferred embodiment is the monoclonal antibody G250 produced by the hybridoma cell line DSM ACC 2526. The antibody cG250 is an IgG1 kappa light chain chimeric version of an originally murine monoclonal antibody mG250.

In particularly preferred embodiments, the antibody for binding to CAIX is one that is described in WO 2021/000017 (eg., GmAb and radiolabelled conjugated form DOTA-GmAb), the contents of which are hereby incorporated by reference. Radioimmunoconjugates for binding to CAIX are also described in WO 2021/000017, as are radioimmunoconjugates for binding to CAIX, and which have a reduced serum half-life (i.e., increased rate of serum clearance).

In a particularly preferred embodiment, the antibody for binding to CAIX comprises:

FR1-CDR1-FR2-CDR2-FR3-CDR3-FR4

and

FR1a-CDR1a-FR2a-CDR2a-FR3a-CDR3a-FR4a

wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 2 below. Preferably, the framework regions have an amino acid sequence also as described in Table 2 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

Preferably the antibody is in the format: FR1-CDR1-FR2-CDR2-FR3-CDR3-FR4-linker-FR1a-CDR1a-FR2a-CDR2a-FR3a-CDR3a-FR4a; or FR1a-CDR1a-FR2a-CDR2a-FR3a-CDR3a-FR4a-FR1-CDR1-FR2-CDR2-FR3-CDR3-FR4.

As defined herein, the linker may be a chemical, one or more amino acids, or a disulphide bond formed between two cysteine residues. In preferred embodiments, the linker is comprised of one or more amino acid residues.

The antibody for specifically binding to CAIX preferably comprises an antigen binding site that consists essentially of or consists of an amino acids sequence of (in order of N to C terminus or C to N terminus) SEQ ID NO: 52, 68, 84, 100 or 116.

In a further embodiment, the antibody that specifically binds to CAIX comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 49, 65, 81, 97 or 113, a CDR2 comprising a sequence at least about 80%, at least 85%, at least 90%, at least 92%, at least 95%, at least 97%, at least 99% identical to a sequence set in SEQ ID NO:50, 66, 82, 98 or 114, and a CDR3 comprising a sequence at least about 80%, at least 85%, at least 90%, at least 92%, at least 95%, at least 97%, at least 99% identical to a sequence set forth in SEQ ID NO: 51, 67, 83, 99 or 115;
(ii) a VH comprising a sequence at least about 95% or 96% or 97% or 98% or 99% identical to a sequence set forth in SEQ ID NO: 52, 68, 84, 100 or 116;
(iii) a VL comprising a CDR1 comprising a sequence at least about 80%, at least 85%, at least 90%, at least 92%, at least 95%, at least 97%, at least 99% identical to a sequence set forth in SEQ ID NO: 129, 145, 161, 177, 193, or 209, a CDR2 comprising a sequence at least about 80%, at least 85%, at least 90%, at least 92%, at least 95%, at least 97%, at least 99% identical to a sequence set forth in SEQ ID NO: 130, 146, 162, 178, 194 or 210 and a CDR3 comprising a sequence at least about 80%, at least 85%, at least 90%, at least 92%, at least 95%, at least 97%, at least 99% identical to a sequence set forth in SEQ ID NO: 131, 147, 163, 179, 195, or 211;
(iv) a VL comprising a sequence at least about 95% identical to a sequence set forth in SEQ ID NO:132, 148, 164, 180, 196 or 212;
(v) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115;
(vi) a VH comprising a sequence set forth in SEQ ID NO: 52, 68, 84, 100 or 116; (vii) a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211;
(viii) a VL comprising a sequence set forth in SEQ ID NO: 132, 148, 164, 180, 196 or 212;
(ix) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115; and a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211; or
(x) a VH comprising a sequence set forth in SEQ ID NO: 52, 68, 84, 100 or 116 and a VL comprising a sequence set forth in SEQ ID NO: 132, 148, 164, 180, 196 or 212.

Preferably, the heavy chain constant region comprises amino acid substitutions at both His310 and His435. The antibody may also comprise amino acid substitutions at residues equivalent to Ser228 and Leu235 of the constant heavy chain region.

Preferably, the antibody comprises a heavy chain constant region comprising the sequence as set forth in any one of SEQ ID NOs: 225 to 228, preferably as set forth in SEQ ID NO: 226.

In a still further embodiment, the antibody preferably comprises a heavy chain comprising the sequence set forth in any one of SEQ ID NOs: 230 to 233, preferably as set forth in SEQ ID NO: 231.

In any embodiment, the antibody comprises a light chain constant region comprising the amino acid sequence as set forth in SEQ ID NO: 229. Preferably, the antibody comprises a light chain comprising the amino acid sequence as set forth in SEQ ID NO:234.

In a particular preferred embodiment, the antibody comprises the sequence set forth in SEQ ID NO:231 and the sequence set forth in SEQ ID NO: 234.

### Antibodies for Binding PSMA

As used herein, PSMA refers to “prostate specific membrane antigen”.

Preferably, an antibody for binding to PSMA interacts with, e.g., binds to, the extracellular domain of PSMA, e.g., the extracellular domain of human PSMA located at about amino acids 44-750 of human PSMA (amino acid residues correspond to the human PSMA sequence disclosed in U.S. Pat. No. 5,538,866). In some embodiments, the antibody binds to a dimer of PSMA, e.g., the agent binds to a portion of PSMA exposed in both a dimer of PSMA and a monomer of PSMA, or the agent binds to a portion of PSMA exposed on a PSMA dimer but not a PSMA monomer. Preferably, the interaction, e.g., binding, occurs with high affinity and specificity. Preferably, the PSMA binding agent treats, e.g., ablates or kills, a cell, e.g., a PSMA-expressing cell (e.g., a cancerous cell or a vascular endothelial cell). The mechanism by which the PSMA binding agent treats, e.g., ablates or kills, the cell is not critical to the practice of the invention. In some embodiments, the PSMA binding agent may bind to and be internalized with the PSMA expressed in the cells and/or vascular endothelial cells proximate to the cells. In those embodiments, the binding agent can be used to target a second moiety, e.g., a cytotoxic agent, to the cell. In other embodiments, the PSMA binding agent may mediate host mediated-killing, e.g., complement- or ADCC-mediated killing, of the cell and/or the vascular cell proximate thereto, upon binding to the extracellular domain of PSMA. The cell can be killed directly by the PSMA binding agent binding directly to the cell (e.g., to a cancerous cell) or to vascular endothelial cells proximate thereto. Alternatively, the PSMA binding agent can treat, e.g., kill or ablate, or otherwise change the properties of the vascular endothelial cells to which it binds so that blood flow to the cells proximate thereto is reduced, thereby causing the proximate cells to be killed or ablated.

An “anti-PSMA antibody” is an antibody that interacts with (e.g., binds to) PSMA, preferably human PSMA protein. The antibody can be any PSMA-specific antibody (e.g., a monospecific, or a recombinant or modified antibody), and includes antigen-binding fragments thereof.

Anti-PSMA antibodies, and fragments thereof are known, including antibodies that binds to PSMA, preferably human PSMA, with high affinity and specificity. In some embodiments, the antibodies are those having one or more complementarity determining regions (CDRs) from a J591, J415, J533 or E99 antibody or from an antibody which competes with or has an overlapping epitope with one of these antibodies. Antibody J591 is described in Liu et al., Cancer Res 1997; 57: 3629-34. In any embodiment of the invention, the anti-PSMA binding antibody or antigen binding fragment thereof may have a light chain variable region comprising one or more complementarity determining regions (CDRs) from a monoclonal antibody selected from the group consisting of J591, J415, J533 and E99 or from an antibody which competes with or has an overlapping epitope with one of these antibodies, and/or a heavy chain variable region comprising one or more CDRs from a monoclonal antibody selected from the group consisting of J591, J415, J533 and E99 or from an antibody which competes with or has an overlapping epitope with one of these antibodies. In some embodiments, the antibody or antigen binding portion thereof comprises all six CDRs from murine J591, or all six CDRs from murine J415. In other embodiments, the antibodies are those having one or more complementarity determining regions (CDRs) from a 4A3, 7F12, 8A11, 8C12, 16F9 026 or PSMA 4.40 antibody or from an antibody which competes with or has an overlapping epitope with one of these antibodies, e.g., having a light chain variable region comprising one or more complementarity determining regions (CDRs) from a monoclonal antibody selected from the group consisting of 4A3, 7F12, 8A11, 8C12, 16F9 026 and PSMA 4.40 or from an antibody which competes with or has an overlapping epitope with one of these antibodies, and/or a heavy chain variable region comprising one or more CDRs from a monoclonal antibody selected from the group consisting of 4A3, 7F12, 8A11, 8C12, 16F9 026 and PSMA mAb 4.40 or from an antibody which competes with or has an overlapping epitope with one of these antibodies. In some embodiments, the antibody or antigen binding portion thereof comprises all six CDRs from one of the aforementioned antibodies.

Further antibodies for binding PSMA, and contemplated for use according to the invention include the antibodies disclosed in US 20190022205 (“10B3” antibody), the contents of which, particular antibody sequences thereof, are herein disclosed by reference.

In some embodiments, the anti-PSMA monospecific antibody is a monoclonal, chimeric, CDR-grafted, humanized, e.g., a humanized mouse antibody, deimmunized, e.g., a deimmunized mouse antibody, or human antibody or an antigen-binding fragment thereof. The anti-PSMA antibody (e.g., recombinant or modified antibodies) can be full-length (e.g., an IgG (e.g., an IgGI, IgG2, IgG3, IgG4), IgM, IgA (e.g., IgAI, IgA2), IgD, and IgE, but preferably an IgG) or can include only an antigen-binding fragment (e.g., a Fab, F(ab′)2 or scFv fragment, or one or more CDRs). An antibody, or antigen-binding fragment thereof, can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. A preferred anti-PSMA antibody includes a heavy and/or light chain constant region substantially from a human antibody, e.g., a human IgG1 constant region or a portion thereof. In some embodiments, the anti-PSMA antibodies are human antibodies.

The antibody (or fragment thereof) can be a murine or a human antibody. Examples of murine monoclonal antibodies that can be used include a E99, J415, J533 and J591 antibody, which are produced by hybridoma cell lines having an ATCC Accession Number HB-12101, HB-12109, HB-12127, and HB-12126, respectively. Also within the scope of the invention are methods and compositions using antibodies, or antigen-binding fragments thereof, which bind overlapping epitopes of, or competitively inhibit, the binding of an anti-PSMA antibody disclosed herein to PSMA, e.g., antibodies which bind overlapping epitopes of, or competitively inhibit, the binding of one or more of monoclonal antibody E99, J415, J533, J591, 4A3, 7F12, 8A11, 8C12, 16F9 026 or PSMA 4.40 to PSMA. Any combination of anti-PSMA antibodies can be used, e.g., two or more antibodies that bind to different regions of PSMA, e.g., antibodies that bind to two different epitopes on the extracellular domain of PSMA.

In some embodiments, the binding agent is an anti-PSMA antibody that binds to all or part of the epitope of an antibody described herein, e.g., a J591, E99, J415, J533, 4A3, 7F12, 8A11, 8C12, 16F9 026 and PSMA 4.40 antibody. The anti-PSMA antibody can inhibit, e.g., competitively inhibit, the binding of an antibody described herein, e.g., a J591, E99, J415, J533, 4A3, 7F12, 8A11, 8C12, 16F9 026 and PSMA 4.40 antibody, to human PSMA. An anti-PSMA antibody may bind to an epitope, e.g., a conformational or a linear epitope, which epitope when bound prevents binding of an antibody described herein, a J591, E99, J415, J533, 4A3, 7F12, 8A11, 8C12, 16F9 026 and PSMA 4.40 antibody. The epitope can be in close proximity spatially or functionally associated, e.g., an overlapping or adjacent epitope in linear sequence or conformationally to the one recognized by the J591, E99, J415, J533, 4A3, 7F12, 8A11, 8C12, 16F9 026 or PSMA 4.40 antibody.

In one embodiment, the anti-PSMA antibody binds to an epitope located wholly or partially within the region of about amino acids 120 to 500, e.g., 130 to 450, 134 to 437, or 153 to 347, of human PSMA. Typically, the epitope includes at least one glycosylation site, e.g., at least one N-linked glycosylation site (e.g., the N-linked glycosylation site located at about amino acids 190-200, preferably at about amino acid 195, of human PSMA).

In other embodiments, the antibody (or antigen-binding fragment thereof) is a recombinant or modified anti-PSMA antibody chosen from, e.g., a chimeric, a CDR-grafted, a humanized, a deimmunized, or an in vitro generated antibody (or an antigen-binding fragment thereof). As discussed herein, the modified antibodies can be CDR-grafted, humanized, deimmunized, or more generally, antibodies having CDRs from a non-human antibody, e.g., murine J591, J415, J533 or E99 antibody and a framework that is selected as less immunogenic in humans, e.g., less antigenic than the murine framework in which a murine CDR naturally occurs. In one embodiment, a modified antibody is a deimmunized anti-PSMA antibody, e.g., a deimmunized form of E99, J415, J533 or J591 (e.g., a deimmunized form of an antibody produced by a hybridoma cell line having an ATCC Accession Number HB-12101, HB-12109, HB-12127 and HB-12126, respectively).

Typically, the antibody is a deimmunized form of J591 or J415 (referred to herein as “deJ591” or “deJ415” respectively). Most preferably, the antibody is a deimmunized form of J591. The antibody can be a human antibody, e.g., a human antibody made in a non-human animal, e.g., a mouse.

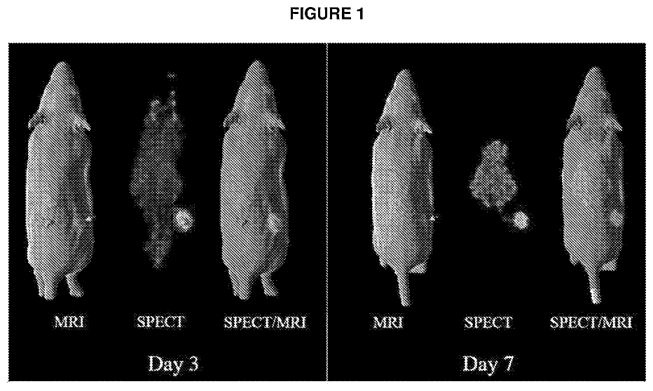

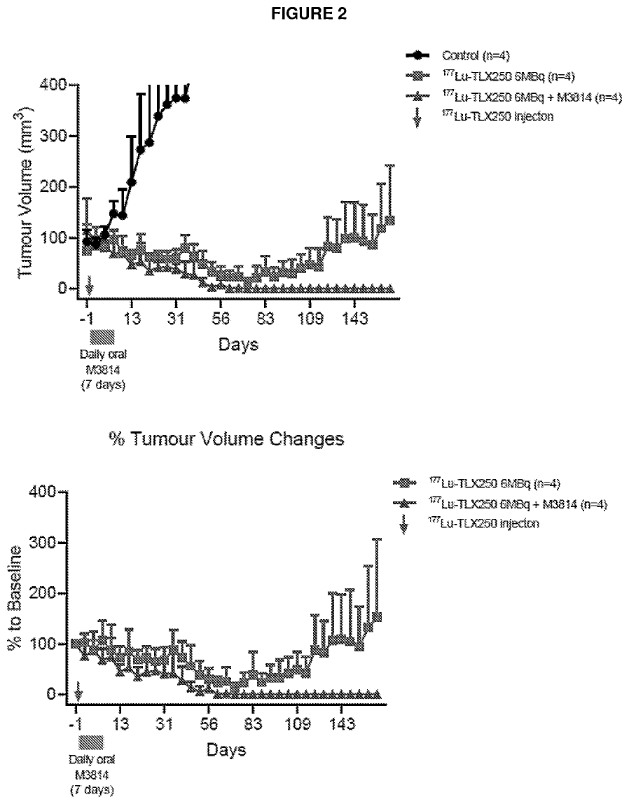

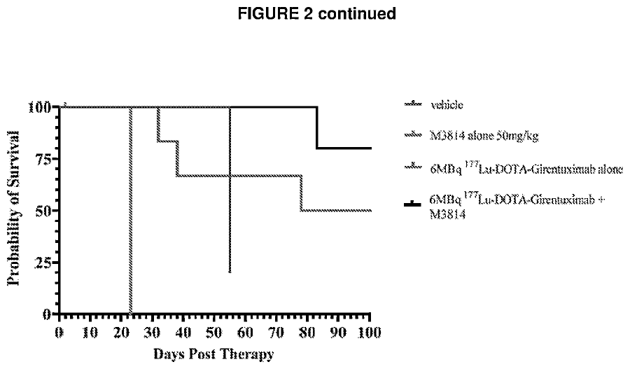

The antibody or antigen-binding fragment thereof can have at least one, two and preferably three CDRs from: the heavy chain variable region of murine J591 (as defined in SEQ ID NO: 1, 2, and 3, and depicted in FIG. 1A of US20060088539, incorporated herein by reference); and the light chain variable region of murine J591 (see SEQ ID NO:4, 5 and 6, depicted in FIG. 1B of US20060088539, incorporated herein by reference). The antibody or antigen-binding fragment thereof can have the heavy variable and light chains of the J591 antibody, or any modified form thereof, as described in US20060088539, FIGS. 1A and 1B. The antibody or antigen-binding fragment thereof can have the heavy variable and light chains of a deimmunised J591 antibody, or any modified form thereof, as described in US20060088539, FIGS. 2A and 2B.

As used herein, ANT4044 and ANT4044-A2 refer to humanised and affinity matured humanised forms, respectively, of the J591 antibody; the sequences of which are set forth in Table 1, herein.

In particularly preferred embodiments, the antibody for binding to PSMA is one that is described in WO 2021/000017, the contents of which are hereby incorporated by reference. Radioimmunoconjugates for binding to PSMA are also described in WO 2021/000017.

In preferred embodiments, wherein said antibody binds specifically to prostate specific membrane antigen (PSMA), the antibody comprises:

FR1-CDR1-FR2-CDR2-FR3-CDR3-FR4; and

FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 1 below. Preferably, the framework regions have an amino acid sequence also as described in Table 1 below, including amino acid variation at particular residues which can be determined by aligning the various framework regions derived from each antibody. The invention also includes where CDR1, CDR2 and CDR3 are sequences from the VH, CDR1a, CDR2a and CDR3a are sequences from VL, or where CDR1, CDR2 and CDR3 are sequences from the VL, CDR1a, CDR2a and CDR3a are sequences from VH.

Preferably the antibody is in the format: FR1-CDR1-FR2-CDR2-FR3-CDR3-FR4-linker-FR1a-CDR1a-FR2a-CDR2a-FR3a-CDR3a-FR4a; or FR1a-CDR1a-FR2a-CDR2a-FR3a-CDR3a-FR4a-FR1-CDR1-FR2-CDR2-FR3-CDR3-FR4.

As defined herein, the linker may be a chemical, one or more amino acids, or a disulphide bond formed between two cysteine residues. In preferred embodiments, the linker is comprised of one or more amino acid residues.

More preferably, the antibody for binding PSMA comprises an antigen binding site that consists essentially of or consists of an amino acids sequence of (in order of N to C terminus or C to N terminus) SEQ ID NO: 4 or 20 and/or SEQ ID NO: 36

In a further embodiment, the antibody that specifically binds to PSMA comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 1, 17 or 244, a CDR2 comprising a sequence at least about 80%, at least 85%, at least 90%, at least 92%, at least 95%, at least 97%, at least 99% identical to a sequence set in SEQ ID NO: 2 or 18, and a CDR3 comprising a sequence at least about 80%, at least 85%, at least 90%, at least 92%, at least 95%, at least 97%, at least 99% identical to a sequence set forth in SEQ ID NO: 3 or 19;
(ii) a VH comprising a sequence at least about 95% or 96% or 97% or 98% or 99% identical to a sequence set forth in SEQ ID NO: 4, 20 or 245;
(iii) a VL comprising a CDR1 comprising a sequence at least about 80%, at least 85%, at least 90%, at least 92%, at least 95%, at least 97%, at least 99% identical to a sequence set forth in SEQ ID NO: 33, a CDR2 comprising a sequence at least about 80%, at least 85%, at least 90%, at least 92%, at least 95%, at least 97%, at least 99% identical to a sequence set forth in SEQ ID NO: 34 and a CDR3 comprising a sequence at least about 80%, at least 85%, at least 90%, at least 92%, at least 95%, at least 97%, at least 99% identical to a sequence set forth in SEQ ID NO: 35;
(iv) a VL comprising a sequence at least about 95% identical to a sequence set forth in SEQ ID NO:36 or 246;
(v) a VH comprising a CDR1 comprising a sequence set forth in SEQ ID NO: 1, 17 or 244, a CDR2 comprising a sequence set forth between in SEQ ID NO: 2 or 18 and a CDR3 comprising a sequence set forth in SEQ ID NO: 3 or 19;
(vi) a VH comprising a sequence set forth in SEQ ID NO: 4, 20 or 245; (vii) a VL comprising a CDR1 comprising a sequence set SEQ ID NO: 33, a CDR2 comprising a sequence set forth in SEQ ID NO: 34 and a CDR3 comprising a sequence set forth in SEQ ID NO: 45;
(viii) a VL comprising a sequence set forth in SEQ ID NO: 36 or 246;
(ix) a VH comprising a CDR1 comprising a sequence set forth in SEQ ID NO: 1 or 17, a CDR2 comprising a sequence set forth between in SEQ ID NO: 2 or 18 and a CDR3 comprising a sequence set forth in SEQ ID NO: 3 or 19; and a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35; or
(x) a VH comprising a sequence set forth in SEQ ID NO: 4 or 20 or 245 and a VL comprising a sequence set forth in SEQ ID NO: 36 or 246.

In preferred embodiments, the antibody or antigen binding fragment thereof comprises the heavy chain CDRs having the amino acid sequences as set forth in SEQ ID NO: 244, 18 and 19; and the light chain CDRs as set forth in SEQ ID NOs: 33, 34 and 35.

In further preferred embodiments, the antibody or antigen binding fragment thereof comprises the heavy chain CDRs having the amino acid sequences as set forth in SEQ ID NO: 1, 18 and 19; and the light chain CDRs as set forth in SEQ ID NOs: 33, 34 and 35.

In certain embodiments, the heavy chain constant region comprises amino acid substitutions at both His310 and His435. The antibody may also comprise amino acid substitutions at residues equivalent to Ser228 and Leu235 of the constant heavy chain region.

In any embodiment, the antibody comprises a heavy chain constant region that comprises the amino acid sequence as set forth in any one of SEQ ID NOs: 235 to 237, preferably wherein the heavy chain constant region comprises the sequence set forth in SEQ ID NO:236.

In still a further embodiment, the heavy chain of the antibody comprises the sequence set forth in any one of SEQ ID NOs: 239 to 242, preferably as set forth in SEQ ID NO: 239, more preferably SEQ ID NO: 245.

Still further, in preferred embodiments, the light chain constant region of the antibody comprises the sequence as set forth in SEQ ID NO: 238. More preferably, the antibody comprises a light chain comprising the amino acid sequence as set forth in SEQ ID NO:243. Most preferably, the antibody comprises a light chain comprising the amino acid sequence as set forth in SEQ ID NO:246.

In a particularly preferred embodiment, the antibody comprises the amino acid sequence set forth in SEQ ID NO: 239 and the sequence set forth in SEQ ID NO: 243.

### Antibodies for Binding to Other Tumour-Associated or Tumour-Specific Antigens

It will be appreciated that in accordance with any aspect of the invention, the radioimmunoconjugate comprises an antibody or fragment thereof for binding to an antigen expressed by the cancer requiring treatment. It is well within the purview of a person skilled in the art to identify a suitable antibody for use in treating any given cancer.

In the context of the present invention, “tumour antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumour antigens are proteins that are produced by tumour cells that elicit an immune response, particularly T-cell mediated immune responses. The selection of the antigen binding moiety of the invention will depend on the particular type of cancer to be treated, Tumour antigens are well known in the art and include, for example, a glioma-associated antigen, carcinoembryonic antigen (CEA), β-human chorionic gonadotropin, alphafetoprotein (AFP), lectin-reactive AFP, thyroglobulm, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin,

In one embodiment, the tumour antigen comprises one or more antigenic cancer epitopes associated with a malignant tumour. Malignant tumours express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumour-specific idiotype immunoglobulin constitutes a truly tumour-specific immunoglobulin antigen that is unique to the individual tumour. B-cell differentiation antigens such as CD 19, CD20 and CD37 are other candidates for target antigens in B-cell lymphoma.

The type of tumour antigen referred to in the invention may also be a tumour-specific antigen (TSA) or a tumour-associated antigen (TAA). A TSA is unique to tumour cells and does not occur on other cells in the body. A TAA associated antigen is not unique to a tumour cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during foetal development when the immune system is immature and unable to respond or they may be antigens that are normally present at extremely low levels on normal cells but which are expressed at much higher levels on tumour cells.

Non-limiting examples of TSA or TAA antigens include the following: Differentiation antigens such as MART-1/MelanA (MART-1), gp 100 (Pmel 17), tyrosinase, TRP-1, TRP-2 and tumour-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumour antigens resulting from chromosomal translocations; such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p 180erbB-3, c-met, nm-23H 1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotem, beta-HCG, BCA225, BTAA, CA 125, CA 15-3\CA 27.29\BCAA, CA 195, CA 242, CA-50, CAM43, CD68\P 1, CO-029, FGF-5, G250, Ga733\EpCAM, HTgp-175, M344, MA-50, MG7-Ag, MOV 18, NB/70K, NY-CO-1, RCAS 1, SDCCAG16, TA-90\Mac-2 binding protein\cyclophilin C-associated protein, TAAL6, TAG72, TLP, and TPS.

In further embodiments, the antibody for use in the radioimmunoconjugate may be modified so as to reduce the serum half-life of the antibody. This can be accomplished by modifying the antibody to have reduced FcRn binding affinity, such as described in WO 2021/000017.

In certain preferred embodiments, the one or more amino acid substitutions may be at one or more of residues His310, His433, His435, His436, or IIe253 of IgG. Preferably, the amino acid substitutions comprise a substitution in the heavy chain constant region at positions His310 or at His435. More preferably, the amino acid substitutions that reduce the affinity of the antibody for FcRn are at both His310 and His435.

In certain embodiments, the modified antibody retains the ability to bind to one or more Fc-gamma receptors and accordingly, in certain embodiments the modified antibody retains the ability to stimulate effector responses (including ADCC).

In alternative embodiments, the one or more amino acid modifications which reduce the affinity for the FcRn receptor also reduce the affinity for the Fc gamma receptors. The modified antibody may further comprise one or more amino acid substitutions compared a wild-type antibody of the class IgG, wherein the amino acid substitutions further reduce the affinity of the antibody for one or more Fc gamma receptors.

In a further embodiment, the modified antibody further comprises one or more amino acid substitutions compared a wild-type antibody of the class IgG, wherein the amino acid substitutions increase the stability of the CH1-CH2 hinge region in the modified antibody compared to a wild-type antibody of the class IgG.

The modified antibody of class IgG with reduced FcRn binding affinity compared to an unmodified antibody of class IgG may be any antibody that is useful for targeting a diagnostic or therapeutic agent to a biological site. The antibody may be of any IgG class, including IgG1 (human or murine), IgG2, IgG4, murine IgG2a.

### Linkers and Crosslinking

In any aspect of the invention, an antibody or fragment thereof, is conjugated to a radionuclide. The radionuclide may be conjugated to the antibody directly or indirectly, e.g. by halogenation of amino acid residues. Preferably, the radionuclide agent is indirectly conjugated to the antibody by way of a linker or chelator moiety.

In another example, the antibody is conjugated to a bifunctional linker, for example, bromoacetyl, thiols, succinimide ester, TFP ester, a maleimide, or using any amine or thiol-modifying chemistry known in the art.

The term “chelate” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a therapeutic moiety, targeting moiety, or cross-linking group.

Examples of suitable chelating moieties include, but are not limited to, DOTA (1,4,7,10-tetraazacyclododecane-1,4,7,10-tetraacetic acid), DOTMA (1R,4R,7R,10R)-α, α′, α″, α′″-tetramethyl-1.4.7.10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), D03AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N′″,N″″-pentaacetic acid), H4octapa (N,N′-bis(6-carboxy-2-pyridylmethyl)-ethylenediamine-N,N′-diacetic acid), H2dedpa (1,2-[[6-(carboxy)-pyridin-2-yl]-methylamino]ethane), H6phospa (N,N′-(methylenephosphonate)-N,N′-[6-(methoxycarbonyl)pyridin-2-yl]-methyl-1, 2-diaminoethane), TTHA (triethylenetetramine-N,N,N′,N″,N′″, N″″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), HOPO (octadentate hydroxypyridinones), or porphyrins.

In some embodiments, radioimmunoconjugates comprise a metal complex of a chelating moiety. For example, chelating groups may be used in metal chelate combinations with metals, such as manganese, iron, and gadolinium and isotopes (e.g., isotopes in the general energy range of 60 to 4,000 keV), such as any of the radioisotopes and radionuclides discussed herein.

In some embodiments, radioimmunoconjugates comprise a cross-linking group. A cross-linking group is a reactive group that is able to join two or more molecules by a covalent bond. Cross-linking groups may be used to attach the linker and chelating moiety to a therapeutic or targeting moiety. Cross-linking groups may also be used to attach the linker and chelating moiety to a target in vivo. In some embodiments, the cross-linking group is an amino-reactive, methionine reactive or thiol-reactive cross-linking group, or a sortase-mediated coupling.

In some embodiments, the amino-reactive or thiol-reactive cross-linking group comprises an activated ester such as a hydroxysuccinimide ester, 2,3,5,6-tetrafluorophenol ester, 4-nitrophenol ester or an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine. In some embodiments, the sortase recognition sequence may comprise of a terminal glycine-glycine-glycine (GGG) and/or LPTXG amino acid sequence, where X is any amino acid. A person having ordinary skill in the art will understand that the use of cross-linking groups is not limited to the specific constructs disclosed herein, but rather may include other known cross-linking groups.

### Additional Therapeutic Agents

In any embodiment, the methods of the invention include a further step (iii) of administering an anti-proliferative agent, a radiation sensitizer, or an immunoregulatory or immunomodulatory agent.

As used herein an “anti-proliferative agent,” refers to any anti-cancer chemotherapeutic agent. The term “anti-proliferative agent” may be used interchangeably with the terms “antineoplastic” or “cytotoxic”. e anti-proliferative agent may be an alkylating agent, a platinum agent, an anti-metabolite, a topoisomerase inhibitor, an anthracycline antibiotic, an antimitotic agent, an aromatase inhibitor, a thymidylate synthase inhibitor, a DNA antagonist, a farnesyltransferase inhibitor, a pump inhibitor, a histone acetyltransferase inhibitor, a metalloproteinase inhibitor, a ribonucleoside reductase inhibitor, a TNFα agonist/antagonist, an endothelin A receptor anatagonist, a kinase inhibitor

Such agents may include organo-platinum derivatives, naphtoquinone and benzoquinone derivatives, chrysophanic acid and anthroquinone derivatives thereof.

As used herein an “immunoregulatory agent” or “immunomodulatory agent,” or “immunomodulatory” as used interchangeably herein, refers to any immuno-modulator, including one selected from the group consisting of: interferon, oncaphage, nivolumab, abatacept, pembrolizumab, ipilimumab, and atezolizumab.

As used herein, a “radiation sensitizer” refers to any agent that increases the sensitivity of cancer cells to radiation therapy. Radiation sensitizers may include, but are not limited to, 5-fluorouracil, analogs of platinum (e.g., cisplatin, carboplatin, oxaliplatin), gemcitabine, EGFR antagonists (e.g., cetuximab, gefitinib), farnesyltransferase inhibitors, COX-2 inhibitors, bFGF antagonists, and VEGF antagonists.

As used herein, the term “chemotherapeutic agent” refers to chemical compounds that are effective in inhibiting tumour growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; cally statin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancrati statin; a sarcodictyin; spongistatin; nitrogen mustards such as chlorambucil, chlomaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2, 2′, 2″-trichlorotriethylamine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumours such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In any aspect, the method includes a further step of administering an immunomodulatory agent. The immunomodulatory may be an immune checkpoint modulator, preferably one selected from: an inhibitor of PD-1, PD-L1 and CTLA-4 or any other immune checkpoint inhibitor described herein.

Optionally, the immune checkpoint inhibitor is an inhibitor of PD-1 selected from: pembrolizumab nivolumab, cemiplimab, spartalizumab, camrelizumab, sintilimab, tislelizuma, toripalimab, dostarlimab, INCMGA00012, AMP-224 and AMP-514.

Optionally, the immune checkpoint inhibitor is an inhibitor of PD-L1 selected from: atezolizumab, avelumab, durvalumab, KN035, CK-301, AUNP12, CA-170, and BMS-986189.

The immune checkpoint inhibitor may be an inhibitor of CTLA-4, selected from: ipilimumab and tremelimumab.

### Indications to be Treated

The present invention is directed to methods for treating diseases or conditions characterised by aberrant cell function. It will be appreciated that typically, such diseases and conditions include cancers, but may also include other proliferative conditions characterised by aberrant cell growth.

As used herein, the term “cancer” refers to a malignant growth or tumour resulting from an uncontrolled division of cells. The term “cancer” includes primary tumours and metastatic tumours and typically refers to any disease caused by the proliferation of malignant neoplastic cells, such as tumours, neoplasms, carcinomas, sarcomas, leukaemias, and lymphomas. A “solid tumour cancer” is a cancer comprising an abnormal mass of tissue, e.g., sarcomas, carcinomas, and lymphomas. A “haematological cancer” or “liquid cancer,” as used interchangeably herein, is a cancer present in a body fluid, e.g., lymphomas and leukaemias.

In any embodiment herein, the cancer may be a metastatic cancer.

Examples of cancers which may be treated according to the methods of the present invention include, pre-neoplastic and neoplastic diseases. Broad examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small 20 round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, oesophageal tumours, laryngeal tumours, liver tumours, lung tumours, vaginal tumours and Wilm's tumour.

In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumour, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumour, malignant; thecoma, malignant; granulosa cell tumour, malignant; and roblastoma, malignant; Sertoli cell carcinoma; leydig cell tumour, malignant; lipid cell tumour, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumour, malignant; mullerian mixed tumour; nephroblastoma; hepatoblastoma; carcinosarcoma; mesenchymoma, malignant; brenner tumour, malignant; phyllodes tumour, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumour of bone; ewing's sarcoma; odontogenic tumour, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumour; meningioma, malignant; neurofibrosarcoma; neurilemmoma, malignant; granular cell tumour, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In a particular embodiment, the cancer is kidney cancer. As used herein, the terms “kidney cancer,” “renal cancer,” or “renal cell carcinoma” refer to cancer that has arisen from the kidney. The terms “renal cell cancer” or “renal cell carcinoma” (RCC), as used herein, refer to cancer which originates in the lining of the proximal convoluted tubule. More specifically, RCC encompasses several relatively common histologic subtypes: clear cell renal cell carcinoma, papillary (chromophil), chromophobe, collecting duct carcinoma, and medullary carcinoma. Clear cell renal cell carcinoma (ccRCC) is the most common subtype of RCC. In a particular embodiment, the cancer is a metastatic renal cell carcinoma.

In preferred embodiments, the cancer is one characterised by the expression of CAIX. The cancer characterised by expression of CAIX may be, although should not be construed as limited to: clear cell renal cancer, head and neck cancer, cervical cancer, pancreatic cancer, non-small cell lung cancer, gastro-oesophageal cancer and hepatocellular carcinoma. In particularly preferred embodiments, the cancer characterised by the expression of CAIX is a clear cell renal cancer, optionally a metastatic renal cell cancer. In such embodiments, the radiotherapeutic for use in treating the cancer comprises an antibody for binding to CAIX, preferably one as described herein, most preferably wherein the antibody is conjugated to a beta-emitting radioligand.

In a particular embodiment, the cancer is prostate cancer. As used herein, the terms “prostate cancer,” refers to cancer that has arisen from the prostate. In a particular embodiment, the cancer is a metastatic prostate cancer. In certain embodiments, the cancer is metastatic castration-resistant prostate cancer (mCRPC).

In a particular embodiment, the cancer is characterised by the expression of PSMA and may be selected from: prostate cancer, bladder cancer, testicular-embryonal cancer, neuroendocrine cancer, renal cell carcinoma, and breast cancer. In particularly preferred embodiments, the cancer characterised by the expression of PSMA is a prostate cancer, optionally metastatic prostate cancer, such as metastatic castration-resistant prostate cancer. In such embodiments, the radiotherapeutic for use in treating the cancer comprises an antibody for binding to PSMA, preferably one as described herein, most preferably wherein the antibody is conjugated to a beta-emitting radioligand.

In any embodiment, the cancer to be treated by the combination is most preferably a cancer wherein the cancer cells express DNA-PK, respectively the catalytic subunit of DNA-PKAcs, or, expressed differently, wherein the cancer cells exhibit DNA-PK respectively DNA-PKcs activity.

Other diseases and conditions include various inflammatory conditions. Examples may include a proliferative component. Particular examples include acne, angina, arthritis, aspiration pneumonia, disease, empyema, gastroenteritis, inflammation, intestinal flu, nee, necrotising enterocolitis, pelvic inflammatory disease, pharyngitis, pid, pleurisy, raw throat, redness, rubor, sore throat, stomach flu and urinary tract infections, chronic inflammatory demyelinating polyneuropathy, chronic inflammatory demyelinating polyradiculoneuropathy, chronic inflammatory demyelinating polyneuropathy or chronic inflammatory demyelinating polyradiculoneuropathy.

### Methods of Treatment and Administration

In some disclosed methods, a therapy (e.g., comprising a therapeutic agent) is administered to a subject. In some embodiments, the subject is a mammal, e.g., a human.

In some embodiments, the subject has cancer or is at risk of developing cancer. For example, the subject may have been diagnosed with cancer. The cancer may be a primary cancer or a metastatic cancer. Subjects may have any stage of cancer, e.g., stage I, stage II, stage Ill, or stage IV with or without lymph node involvement and with or without metastases. Provided compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is a solid tumour. The solid tumour cancer may be breast cancer, non-small cell lung cancer, small cell lung cancer, pancreatic cancer, head and neck cancer, prostate cancer, colorectal cancer, sarcoma, adrenocortical carcinoma, neuroendocrine cancer, Ewing's Sarcoma, multiple myeloma, or acute myeloid leukaemia.

In preferred embodiments, the solid tumour cancer may be one that is characterised by the expression of CAIX, such as a clear cell renal cancer, head and neck cancer, cervical cancer, pancreatic cancer, non-small cell lung cancer, gastro-oesophageal cancer and hepatocellular carcinoma. In particularly preferred embodiments, the cancer characterised by the expression of CAIX is a clear cell renal cancer.

In further preferred embodiments, the cancer may be one that is characterised by the expression of PSMA, such as prostate cancer, bladder cancer, testicular-embryonal cancer, neuroendocrine cancer, renal cell carcinoma, and breast cancer. In a particular embodiment, the cancer is a metastatic prostate cancer. In certain embodiments, the cancer is metastatic castration-resistant prostate cancer (mCRPC).

In some embodiments, the cancer is a non-solid (e.g., liquid (e.g., hematologic)) cancer.

The term an “effective amount” of an agent (e.g., any of the foregoing conjugates), as used herein, is that amount sufficient to effect beneficial or desired results, such as clinical results, and, as such, an “effective amount” depends upon the context in which it is being applied.

As used herein, the term “administered in combination,” “combined administration,” or “co-administered” means that two or more agents are administered to a subject at the same time or within an interval such that there may be an overlap of an effect of each agent on the patient. Thus, two or more agents that are administered in combination need not be administered together. In some embodiments, they are administered within 90 days (e.g., within 80, 70, 60, 50, 40, 30, 20, 10, 5, 4, 3, 2, or 1 day(s)), within 28 days (e.g., with 14, 7, 6, 5, 4, 3, 2, or 1 day(s), within 24 hours (e.g., 12, 6, 5, 4, 3, 2, or 1 hour(s), or within about 60, 30, 15, 10, 5, or 1 minute of one another. In some embodiments, the administrations of the agents are spaced sufficiently closely together such that a combinatorial effect is achieved.

In preferred embodiments, the radioimmunotherapeutic is administered prior to the administration of the DNA-PKi. Preferably, the DNA-PKi is administered within 28 days (e.g., with 14, 7, 6, 5, 4, 3, 2, or 1 day(s) of the radioimmunotherapeutic, most preferably within 1, 2, 3, 4 or 5 days of the radioimmunotherapeutic, especially within 1 day of the radioimmunotherapeutic.

As used herein, “administering” an agent to a subject includes contacting cells of said subject with the agent.

The present disclosure provides combination therapies in which the amounts of each therapeutic may or may not be, on their own, therapeutically effective. For example, provided are methods comprising administering a first therapy and a second therapy in amounts that together are effective to treat or ameliorate a disorder, e.g., cancer. In some embodiments, at least one of the first and second therapy is administered to the subject in a lower effective dose. In some embodiments, both the first and the second therapies are administered in lower effective doses.

The term “lower effective dose,” when used as a term in conjunction with an agent (e.g., a therapeutic agent) refers to a dosage of the agent which is effective therapeutically in the combination therapies of the invention and which is lower than the dose which has been determined to be effective therapeutically when the agent is used as a monotherapy in reference experiments or by virtue of other therapeutic guidance.

In some embodiments, the first therapy comprises a radioimmunoconjugate and the second therapy comprises a DNA-PK inhibitor (DNA-PKi).

In some embodiments, the first therapy comprises a DNA-PKi and the second therapy comprises a radioimmunoconjugate.

In some embodiments, therapeutic combinations as disclosed herein are administered to a subject in a manner (e.g., dosing amount and timing) sufficient to cure or at least partially arrest the symptoms of the disorder and its complications. In the context of a single therapy (a “monotherapy”), an amount adequate to accomplish this purpose is defined as a “therapeutically effective amount,” an amount of a compound sufficient to substantially improve at least one symptom associated with the disease or a medical condition. The “therapeutically effective amount” typically varies depending on the therapeutic. For known therapeutic agents, the relevant therapeutically effective amounts may be known to or readily determined by those of skill in the art.

For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual. For example, a treatment may be therapeutically effective if it causes a cancer to regress or to stop or to slow the cancer's growth.

The dosage regimen (e.g., amounts of each therapeutic, relative timing of therapies, etc.) that is effective for these uses may depend on the severity of the disease or condition and the weight and general state of the subject. For example, the therapeutically effective amount of a particular composition comprising a therapeutic agent applied to mammals (e.g., humans) can be determined by the ordinarily-skilled artisan with consideration of individual differences in age, weight, and the condition of the mammal.

Therapeutically effective and/or optimal amounts can also be determined empirically by those of skill in the art. Thus, lower effective doses can also be determined by those of skill in the art.

Single or multiple administrations of a composition (e.g., a pharmaceutical composition comprising a therapeutic agent) can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

In some embodiments, compositions (such as compositions comprising radioimmunoconjugates) are administered for radiation treatment planning or diagnostic purposes. When administered for radiation treatment planning or diagnostic purposes, compositions may be administered to a subject in a diagnostically effective dose and/or an amount effective to determine the therapeutically effective dose.

In some embodiments, a first dose of disclosed radioimmunoconjugate or a composition (e.g., pharmaceutical composition) thereof is administered in an amount effective for radiation treatment planning, followed administration of a combination therapy including a conjugate as disclosed herein and another therapeutic.

In the disclosed combination therapy methods, the first and second therapies may be administered sequentially or concurrently to a subject. For example, a first composition comprising a first therapeutic agent and a second composition comprising a second therapeutic agent may be administered sequentially or concurrently to a subject. Alternatively or additionally, a composition comprising a combination of a first therapeutic agent and a second therapeutic agent may be administered to the subject.

In some embodiments, the radioimmunoconjugate is administered in a single dose. In some embodiments, the radioimmunoconjugate is administered more than once. When the radioimmunoconjugate is administered more than once, the dose of each administration may be the same or different.

In some embodiments, the DNA-PKi is administered in a single dose. In some embodiments, the DNA-PKi is administered more than once, e.g., at least twice, at least three times, etc. In some embodiments, the DNA-PKi is administered multiple times according to a regular or semi-regular schedule, e.g., once every approximately two weeks, once a week, twice a week, three times a week, or more than three times a week. When the DNA-PKi is administered more than once, the dose of each administration may be the same or different. For example, the DNA-PKi may be administered in an initial dose amount, and then subsequent dosages of the DNA-PKi may be higher or lower than the initial dose amount.

In some embodiments, the first dose of the DNA-PKi is administered at the same time as the first dose of the radioimmunoconjugate. In some embodiments, the first dose of the DNA-PKi is administered before the first dose of radioimmunoconjugate. In some embodiments, the first dose of the DNA-PKi is administered after the first dose of radioimmunoconjugate. In some embodiments, subsequent doses of the DNA-PKi are administered.

In some embodiments, radioimmunoconjugates (or a composition thereof) and DNA-PKis (or a composition thereof) are administered within 28 days (e.g., within 14, 7, 6, 5, 4, 3, 2, or 1 day(s)) of each other. In various embodiments the DNA-PKi is administered at the same time as radioimmunoconjugate.

In some embodiments, radioimmunoconjugates (or a composition thereof) and DNA-PKis (or a composition thereof) are administered within 90 days (e.g., within 80, 70, 60, 50, 40, 30, 20, 10, 5, 4, 3, 2, or 1 day(s)) of each other. Most preferably, the DNA-PKi is administered within 1, 2, 3, 4, or 5 days, especially within at least 1 day of the radioimmunoconjugate.

In various embodiments, the DNA-PKi is administered multiple times after the first administration of radioimmunoconjugate. For example, the DNA-PKi may be administered each day over the course of the treatment. For example, the DNA-PKi may be administered every day, for at least 7 days, at least 10 days, at least 14 days, at least 21 days, at least 28 days, or longer, across the period of the therapeutic regimen.

For example, according to a particularly preferred treatment protocol, a patient is administered a radioimmunotherapeutic, as described herein on Day 1 of a treatment plan. On Day 2, treatment with a DNA-PKi, preferably M3814, is commenced, wherein the DNA-PKi is administered on a daily basis during the course of the therapeutic regimen. The therapeutic regimen may comprise one or two or more subsequent doses of the radioimmunotherapeutic, for example, at least about 7 days, at least 14 days, at least 21 days or at least 28 days or at least 35 days, at least 42 days or more, after the administration of the first dose of radioimmunotherapy.

In various embodiments, the treatment may comprise more than one treatment cycle including a period of wash-out (“treatment break”) between treatment cycles to allow for haematological values to return normal, or substantially close to normal values. In such embodiments, the treatment may comprise at least two cycles of treatment, optionally a third or fourth cycle, wherein each treatment cycle comprises administration of the radiotherapeutic on the first day of the cycle, followed by administration of the DNA-PKi on subsequent days (eg, commencing on the second, third or fourth days, preferably on the second day) of the treatment cycle, and comprising administration of the DNA-PKi up to at least the 7^{th}, at least the 14^{th} or at least the 21^{st} days of the treatment cycle. The period between the end of the first treatment cycle and the commencement of the second treatment cycle (ie, wherein no DNA-PKi and no radiotherapeutic are administered) may be at least 7 days, at least 14 days, at least 21 days, at least 28 days, at least 35 days, at least 42 days or more. The second treatment cycle may be substantially the same, or identical to the first treatment cycle (eg, comprising administration of the radiotherapeutic on day 1 of the second treatment cycle, followed by administration of the DNA-PKi on subsequent days of the cycle, preferably starting on the second day, and continuing for at least 7 days, at least 14 days, at least 21 days or more.

The second treatment cycle may be different to the first treatment cycle to the extent that the second treatment cycle may comprise administration of the DNA-PKi for a shorter period of time following administration of the radiotherapeutic, or for a longer period of time following administration of the radiotherapeutic.

In any aspect of the invention, the molecular targeted radiotherapeutic is administered at a dose level below the level required for a monotherapy response. This indicates a synergistic effect between the molecular targeted radiotherapeutic and the DNA-PKi. Preferably, the molecular targeted radiotherapeutic is administered at doses of greater than 10%, preferably greater than 20% less radioactivity compared to the monotherapy response (i.e. the therapy which involves administration of the molecular targeted radiotherapeutic only), preferably 20-50% less radioactivity compared to the monotherapy response. In preferred embodiments, the dosage of the radioimmunotherapeutic is at least about 50% of the dosage required for a therapeutic effect when administered as monotherapy.

For example, the radioimmunotherapeutic may be administered to provide a dose of radiation in the order of between about 500 MBq/m² to about 3000 MBq/m², preferably, between about 800 MBq/m² to about 2000 MBq/m², between about 1000 MBq/m² to about 1800 MBq/m², more preferably about 1000 MBq/m² to about 1500 MBq/m², most preferably about 1100 MBq/m² to about 1500 MBq/m² to a subject, especially wherein the radiation is provided in the form of a beta-emitting radionuclide (such as ¹⁷⁷Lutetium or ¹⁸⁸Rhenium).

In another example, the radioimmunotherapeutic may be administered to provide a dose of radiation in the order of between about 10 mCi/m² to about 80 mCi/m², between about 20 mCi/m² to about 60 mCi/m², between about 25 mCi/m² to about 70 mCi/m², between about 20 mCi/m² to about 50 mCi/m², preferably between about 25 mCi/m² to about 40 mCi/m² especially wherein the radiation is provided in the form of a beta-emitting radionuclide (such as ¹⁷⁷Lutetium or ¹⁸⁸Rhenium).

The skilled person will be familiar with methods for converting the above radiation doses in MBq/m² for a standard 1.7 m² adult individual. For example, in some instances, the radioimmunotherapeutic will be administered at an activity of 1887 MBq (equivalent to a 1110 MBq/m² dose in a standard 1.7 m² adult individual); or of 2516 MBq (equivalent to a 1480 MBq/m² dose in a standard 1.7 m² adult individual); or of 3145 MBq (equivalent to a 1850 MBq/m² dose in a standard 1.7 m² adult individual).

In such embodiments, the DNA-PKi may be administered, optionally on a daily basis, at a dose of 0.02-100 mg/kg, preferably 0.02-50 mg/kg bodyweight. The daily dose in particular may be between 0.02 and 100 mg/kg of body weight. The DNA-Pki may be administered at a dose of between 50-400 mg, more preferably 100-200 mg once daily. Alternatively the DNA-PKi may be administered at a dose of between 150-400 mg twice daily (b.i.d).

The DNA-PKi may be administered at a dose of 0.01 mg to 1 g per dosage unit, preferably between 1 to 700 mg, particularly preferably 5 to 200 mg, for instance 50 mg or 100 mg per unit.

In particularly preferred embodiments, the DNA-PKi is M3814 and the dosing regimen is between about 150-600 mg, preferably 200-500 mg, more preferably 300-400 mg, wherein the dose is administered b.i.d., daily.

In the alternative or most preferably in addition to said dose of the molecular targeted radiotherapeutic, the DNA-PKi is administered at a dose level below the maximum tolerated dose level, for instance at a dose of up to 90%, 85%, 80%, 75%, 60%, 65%, 60% or 55% of the maximum tolerated dose level, and/or at least 10%, or 20%, 30%, 40% or 50% of the maximum tolerated dose level of the combination.

In particularly preferred embodiments, the DNA-PKi is M3814 and the dosing regimen is within one of the following ranges: 25 to 600 mg, 50 to 600 mg, 100 to 600 mg, 150-600 mg, 175 to 500 mg, 200-500 mg, 300-400 mg, 50 to 300 mg, 75 to 275 mg, 100 to 250 mg or a combination thereof. In especially preferred embodiments, the afore-stated dose is administered once daily, but may advantageously also be administered twice daily (b.i.d.). M3814 may, for instance, be administered at a dose of 75 mg, 100 mg, 125 mg, 150 mg, 175 mg, 200 mg, 250 mg, 275 mg or 300 mg, 350 or 400 mg preferably once a day, but suitably also b.i.d, Twice daily administration would most preferably be for doses of 300 mg or more.

One advantage of the present invention, and a distinction from EBRT approaches to delivering radiotherapy, is that the radiotherapeutic is not required to be administered each any every day of the treatment protocol. Accordingly, in any embodiment of the invention, the radiotherapeutic may be administered at intervals of approximately once a week, approximately once every two weeks, approximately once every three weeks; approximately once every four weeks, or at a greater dosing interval. In preferred embodiments, the radiotherapeutic is administered in two doses, at least 7 days, at least 10 days, at least 14 days, at least 21 days, or at least 28 days apart, or in 3 doses, at least 7 days, at least 10 days, at least 14 days, at least 21 days, or at least 28 days apart. It will be appreciated that additional dosing may be required. In certain embodiments, a single administration of the radiotherapeutic may be all that is required

Pharmaceutical compositions comprising one or more agents (e.g., radioimmunoconjugate and/or DNA-PKi) can be formulated for use in accordance with disclosed methods and systems in a variety of drug delivery systems. One or more physiologically acceptable excipients or carriers can also be included in the composition for proper formulation. Examples of suitable formulations are found in Remington's Pharmaceutical Sciences, Mack Publishing Company, Philadelphia, PA, 17th ed., 1985. For a brief review of methods for drug delivery, see, e.g., Langer (Science 249:1527-1533, 1990).

Suitable compositions comprising M3814 for oral administration are described in WO 2018/178134, incorporated herein by reference.

### Kits

The present invention further provides for kits comprising one or more components for use in a method of treatment as described herein.

Preferably the kit comprises a molecular targeting radiotherapeutic and a DNA-PK inhibitor for simultaneous, separate or sequential use in the treatment of a hyperplastic or neoplastic disease, as described herein.

The kit may comprise a container (e.g. a bottle) in which there is a mixture of the two components, or the kit may comprise two separate containers which each contain one of the two components.

### Sequence Information

TABLE 1
Summary of amino acid sequences for PSMA-binding antibodies for
use in the invention

| | | SEQ ID | |
|---|---|---|---|
| Antibody ID | Region | NO: | Amino acid or nucleotide sequence |
| ANT4044 Variable | HCDR1 (protein) | 1 | EYTIH |
| Heavy chain | HCDR2 (protein) | 2 | NINPNNGGTTYNQKFED |
| (HuJ591) | HCDR3 (protein) | 3 | GWNFDY |
| | VH (protein) | 4 | EVQLVQSGAEVKKPGASVKVSCKASGYTFTEYTIHW |
| | | | VRQAPGKGLEWIGNINPNNGGTTYNQKFEDRVTITV |
| | | | DKSTSTAYMELSSLRSEDTAVYYCAAGWNFDYWGQ |
| | | | GTTVTVSS |
| | HCDR1 (DNA) | 5 | GAATACACCATCCAC |
| | HCDR2 (DNA) | 6 | AACATTAATCCTAACAATGGTGGTACTACCTACAAC |
| | | | CAGAAGTTCGAGGAC |
| | HCDR3 (DNA) | 7 | GGTTGGAACTTTGACTAC |
| | VH (DNA) | 8 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACTGAATACACCATCC |
| | | | ACTGGGTGAGGCAGGCCCCTGGAAAGGGCCTTGA |
| | | | GTGGATTGGAAACATTAATCCTAACAATGGTGGTA |
| | | | CTACCTACAACCAGAAGTTCGAGGACAGAGTCACA |
| | | | ATCACTGTAGACAAGTCCACCAGCACAGCCTACAT |
| | | | GGAGCTCAGCAGCCTGAGATCTGAGGATACTGCA |
| | | | GTCTATTACTGTGCAGCTGGTTGGAACTTTGACTA |
| | | | CTGGGGCCAAGGCACCACGGTCACCGTCTCCTCA |
| | HFR1 (protein) | 9 | EVQLVQSGAEVKKPGASVKVSCKASGYTFT |
| | HFR2 (protein) | 10 | WVRQAPGKGLEWIG |
| | HFR3 (protein) | 11 | RVTITVDKSTSTAYMELSSLRSEDTAVYYCAA |
| | HFR4 (protein) | 12 | WGQGTTVTVSS |
| | HFR1 (DNA) | 12 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACT |
| | HFR2 (DNA) | 14 | TGGGTGAGGCAGGCCCCTGGAAAGGGCCTTGAGT |
| | | | GGATTGGA |
| | HFR3 (DNA) | 15 | AGAGTCACAATCACTGTAGACAAGTCCACCAGCAC |
| | | | AGCCTACATGGAGCTCAGCAGCCTGAGATCTGAG |
| | | | GATACTGCAGTCTATTACTGTGCAGCT |
| | HFR4 (DNA) | 16 | TGGGGCCAAGGCACCACGGTCACCGTCTCCTCA |
| ANT4044-A2 | HCDR1 (protein) | 17 | EYTIH |
| Variable Heavy | HCDR2 (protein) | 18 | NINPNNGGTTYNQKFED |
| chain | HCDR3 (protein) | 19 | YWLFDY |
| | VH (protein) | 20 | EVQLVQSGAEVKKPGASVKVSCKASGYTFTEYTIHW |
| | | | VRQAPGKGLEWIGNINPNNGGTTYNQKFEDRVTITV |
| | | | DKSTSTAYMELSSLRSEDTAVYYCAAYWLFDYWGQ |
| | | | GTTVTVSS |
| | HCDR1 (DNA) | 21 | GAATACACCATCCAC |
| | HCDR2 (DNA) | 22 | AACATTAATCCTAACAATGGTGGTACTACCTACAAC |
| | | | CAGAAGTTCGAGGAC |
| | HCDR3 (DNA) | 23 | TACTGGCTGTTCGACTAC |
| | VH (DNA) | 24 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACTGAATACACCATCC |
| | | | ACTGGGTGAGGCAGGCCCCTGGAAAGGGCCTTGA |
| | | | GTGGATTGGAAACATTAATCCTAACAATGGTGGTA |
| | | | CTACCTACAACCAGAAGTTCGAGGACAGAGTCACA |
| | | | ATCACTGTAGACAAGTCCACCAGCACAGCCTACAT |
| | | | GGAGCTCAGCAGCCTGAGATCTGAGGATACTGCA |
| | | | GTCTATTACTGTGCAGCTTACTGGCTGTTCGACTA |
| | | | CTGGGGCCAAGGCACCACGGTCACCGTCTCCTCA |
| | HFR1 (protein) | 25 | EVQLVQSGAEVKKPGASVKVSCKASGYTFT |
| | HFR2 (protein) | 26 | WVRQAPGKGLEWIG |
| | HFR3 (protein) | 27 | RVTITVDKSTSTAYMELSSLRSEDTAVYYCAA |
| | HFR4 (protein) | 28 | WGQGTTVTVSS |
| | HFR1 (DNA) | 29 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACT |
| | HFR2 (DNA) | 30 | TGGGTGAGGCAGGCCCCTGGAAAGGGCCTTGAGT |
| | | | GGATTGGA |
| | HFR3 (DNA) | 31 | AGAGTCACAATCACTGTAGACAAGTCCACCAGCAC |
| | | | AGCCTACATGGAGCTCAGCAGCCTGAGATCTGAG |
| | | | GATACTGCAGTCTATTACTGTGCAGCT |
| | HFR4 (DNA) | 32 | TGGGGCCAAGGCACCACGGTCACCGTCTCCTCA |
| ANT4044/ | LCDR1 (protein) | 33 | KASQDVGTAVD |
| ANT4044-A2 | LCDR2 | 34 | WASTRHT |
| Variable Light | (protein) | | |
| chain | LCDR3 (protein) | 35 | QQYNSYPLT |
| | VL (protein) | 36 | DIQMTQSPSTLSASVGDRVTITCKASQDVGTAVDWY |
| | | | QQKPGQAPKLLIYWASTRHTGVPDRFSGSGSGTDFT |
| | | | LTISRLQPEDFAVYYCQQYNSYPLTFGQGTKVDIK |
| | LCDR1 (DNA) | 37 | AAGGCCAGTCAGGATGTGGGTACTGCTGTAGAC |
| | LCDR2 (DNA) | 38 | TGGGCATCCACCCGGCACACT |
| | LCDR3 (DNA) | 39 | CAGCAATATAACAGCTATCCTCTCACG |
| | VL (DNA) | 40 | GACATTCAGATGACCCAGTCTCCCAGCACCCTGTC |
| | | | CGCATCAGTAGGAGACAGGGTCACCATCACTTGCA |
| | | | AGGCCAGTCAGGATGTGGGTACTGCTGTAGACTG |
| | | | GTATCAACAGAAACCAGGGCAAGCTCCTAAACTAC |
| | | | TGATTTACTGGGCATCCACCCGGCACACTGGAGTC |
| | | | CCTGATCGCTTCAGCGGCAGTGGATCTGGGACAG |
| | | | ATTTCACTCTCACCATCAGCAGACTGCAGCCTGAA |
| | | | GACTTTGCAGTTTATTACTGTCAGCAATATAACAGC |
| | | | TATCCTCTCACGTTCGGCCAGGGGACCAAGGTGG |
| | | | ATATCAAA |
| | LFR1 | 41 | DIQMTQSPSTLSASVGDRVTITC |
| | (protein) | | |
| | LFR2 (protein) | 42 | WYQQKPGQAPKLLIY |
| | LFR3 (protein) | 43 | GVPDRFSGSGSGTDFTLTISRLQPEDFAVYYC |
| | LFR4 | 44 | FGQGTKVDIK |
| | (protein) | | |
| | LFR1 | 45 | GACATTCAGATGACCCAGTCTCCCAGCACCCTGTC |
| | (DNA) | | CGCATCAGTAGGAGACAGGGTCACCATCACTTGC |
| | LFR2(DNA) | 46 | TGGTATCAACAGAAACCAGGGCAAGCTCCTAAACT |
| | | | ACTGATTTAC |
| | LFR3 (DNA) | 47 | GGAGTCCCTGATCGCTTCAGCGGCAGTGGATCTG |
| | | | GGACAGATTTCACTCTCACCATCAGCAGACTGCAG |
| | | | CCTGAAGACTTTGCAGTTTATTACTGT |
| | LFR4 | 48 | TTCGGCCAGGGGACCAAGGTGGATATCAAA |
| | (DNA) | | |
| ANT4044/ANT4044- | IgG1 HC | 235 | ASTKGPSVFPLAPSSKSTSGGTAALGCLVKDYFPEP |
| A2_unmodified | (protein) | | VTVSWNSGALTSGVHTFPAVLQSSGLYSLSSVVTVP |
| human IgG1 | | | SSSLGTQTYICNVNHKPSNTKVDKRVEPKSCDKTHT |
| heavy chain | | | CPPCPAPELLGGPSVFLFPPKPKDTLMISRTPEVTCV |
| constant region | | | VVDVSHEDPEVKFNWYVDGVEVHNAKTKPREEQYN |
| | | | STYRVVSVLTVLHQDWLNGKEYKCKVSNKALPAPIEK |
| | | | TISKAKGQPREPQVYTLPPSREEMTKNQVSLTCLVK |
| | | | GFYPSDIAVEWESNGQPENNYKTTPPVLDSDGSFFL |
| | | | YSKLTVDKSRWQQGNVFSCSVMHEALHNHYTQKSL |
| | | | SLSPGK |
| ANT4044/ANT4044- | IgG1 H310A | 236 | ASTKGPSVFPLAPSSKSTSGGTAALGCLVKDYFPEP |
| A2 modified | H435Q HC | | VTVSWNSGALTSGVHTFPAVLQSSGLYSLSSVVTVP |
| human IgG1 | (protein) | | SSSLGTQTYICNVNHKPSNTKVDKRVEPKSCDKTHT |
| heavy chain | | | CPPCPAPELLGGPSVFLFPPKPKDTLMISRTPEVTCV |
| constant region | | | VVDVSHEDPEVKFNWYVDGVEVHNAKTKPREEQYN |
| | | | STYRVVSVLTVLAQDWLNGKEYKCKVSNKALPAPIEK |
| | | | TISKAKGQPREPQVYTLPPSREEMTKNQVSLTCLVK |
| | | | GFYPSDIAVEWESNGQPENNYKTTPPVLDSDGSFFL |
| | | | YSKLTVDKSRWQQGNVFSCSVMHEALHNQYTQKSL |
| | | | SLSPGK |
| ANT4044/ANT4044- | IgG4 S228P | 237 | ASTKGPSVFPLAPCSRSTSESTAALGCLVKDYFPEPV |
| A2_modified | L235E H310A | | TVSWNSGALTSGVHTFPAVLQSSGLYSLSSVVTVPS |
| human IgG4 | H435Q HC | | SSLGTKTYTCNVDHKPSNTKVDKRVESKYGPPCPPC |
| constant chain | (protein) | | PAPEFEGGPSVFLFPPKPKDTLMISRTPEVTCVVVDV |
| region | | | SQEDPEVQFNWYVDGVEVHNAKTKPREEQFNSTYR |
| | | | VVSVLTVLAQDWLNGKEYKCKVSNKGLPSSIEKTISK |
| | | | AKGQPREPQVYTLPPSQEEMTKNQVSLTCLVKGFYP |
| | | | SDIAVEWESNGQPENNYKTTPPVLDSDGSFFLYSRL |
| | | | TVDKSRWQEGNVFSCSVMHEALHNQYTQKSLSLSL |
| | | | GK |
| ANT4044/ANT4044- | Human κ LC | 238 | RTVAAPSVFIFPPSDEQLKSGTASVVCLLNNFYPREA |
| A2 kappa light | constant region | | KVQWKVDNALQSGNSQESVTEQDSKDSTYSLSSTLT |
| chain constant | | | LSKADYEKHKVYACEVTHQGLSSPVTKSFNRGEC |
| region | | | |
| ANT4044 | FcRn-null, IgG1 | 239 | EVQLVQSGAEVKKPGASVKVSCKASGYTFTEYTIHW |
| RADmAb IgG1 | allotype G1m(3) | | VRQAPGKGLEWIGNINPNNGGTTYNQKFEDRVTITV |
| heavy chain | H310A H435Q | | DKSTSTAYMELSSLRSEDTAVYYCAAGWNFDYWGQ |
| (HuX592r) | | | GTTVTVSSASTKGPSVFPLAPSSKSTSGGTAALGCLV |
| | | | KDYFPEPVTVSWNSGALTSGVHTFPAVLQSSGLYSL |
| | | | SSVVTVPSSSLGTQTYICNVNHKPSNTKVDKRVEPKS |
| | | | CDKTHTCPPCPAPELLGGPSVFLFPPKPKDTLMISRT |
| | | | PEVTCVVVDVSHEDPEVKFNWYVDGVEVHNAKTKP |
| | | | REEQYNSTYRVVSVLTVLAQDWLNGKEYKCKVSNK |
| | | | ALPAPIEKTISKAKGQPREPQVYTLPPSREEMTKNQV |
| | | | SLTCLVKGFYPSDIAVEWESNGQPENNYKTTPPVLD |
| | | | SDGSFFLYSKLTVDKSRWQQGNVFSCSVMHEALHN |
| | | | QYTQKSLSLSPGK |
| ANT4044 | FcRn + FcRγ-null, | 240 | EVQLVQSGAEVKKPGASVKVSCKASGYTFTEYTIHW |
| RADmAb IgG4 | IgG4 S228P | | VRQAPGKGLEWIGNINPNNGGTTYNQKFEDRVTITV |
| heavy chain | L235E H310A | | DKSTSTAYMELSSLRSEDTAVYYCAAGWNFDYWGQ |
| | H435Q | | GTTVTVSSASTKGPSVFPLAPCSRSTSESTAALGCLV |
| | | | KDYFPEPVTVSWNSGALTSGVHTFPAVLQSSGLYSL |
| | | | SSVVTVPSSSLGTKTYTCNVDHKPSNTKVDKRVESK |
| | | | YGPPCPPCPAPEFEGGPSVFLFPPKPKDTLMISRTPE |
| | | | VTCVVVDVSQEDPEVQFNWYVDGVEVHNAKTKPRE |
| | | | EQFNSTYRVVSVLTVLAQDWLNGKEYKCKVSNKGLP |
| | | | SSIEKTISKAKGQPREPQVYTLPPSQEEMTKNQVSLT |
| | | | CLVKGFYPSDIAVEWESNGQPENNYKTTPPVLDSDG |
| | | | SFFLYSRLTVDKSRWQEGNVFSCSVMHEALHNQYT |
| | | | QKSLSLSLGK |
| ANT4044-A2 | FcRn-null, IgG1 | 241 | EVQLVQSGAEVKKPGASVKVSCKASGYTFTEYTIHW |
| RADmAb IgG1 | H310A H435Q | | VRQAPGKGLEWIGNINPNNGGTTYNQKFEDRVTITV |
| heavy chain | | | DKSTSTAYMELSSLRSEDTAVYYCAAYWLFDYWGQ |
| | | | GTTVTVSSASTKGPSVFPLAPSSKSTSGGTAALGCLV |
| | | | KDYFPEPVTVSWNSGALTSGVHTFPAVLQSSGLYSL |
| | | | SSVVTVPSSSLGTQTYICNVNHKPSNTKVDKRVEPKS |
| | | | CDKTHTCPPCPAPELLGGPSVFLFPPKPKDTLMISRT |
| | | | PEVTCVVVDVSHEDPEVKFNWYVDGVEVHNAKTKP |
| | | | REEQYNSTYRVVSVLTVLAQDWLNGKEYKCKVSNK |
| | | | ALPAPIEKTISKAKGQPREPQVYTLPPSREEMTKNQV |
| | | | SLTCLVKGFYPSDIAVEWESNGQPENNYKTTPPVLD |
| | | | SDGSFFLYSKLTVDKSRWQQGNVFSCSVMHEALHN |
| | | | QYTQKSLSLSPGK |
| ANT4044-A2 | FcRn + FcRγ-null | 242 | EVQLVQSGAEVKKPGASVKVSCKASGYTFTEYTIHW |
| RADmAb IgG4 | IgG4 S228P | | VRQAPGKGLEWIGNINPNNGGTTYNQKFEDRVTITV |
| heavy chain | L235E H310A | | DKSTSTAYMELSSLRSEDTAVYYCAAYWLFDYWGQ |
| | H435Q | | GTTVTVSSASTKGPSVFPLAPCSRSTSESTAALGCLV |
| | | | KDYFPEPVTVSWNSGALTSGVHTFPAVLQSSGLYSL |
| | | | SSVVTVPSSSLGTKTYTCNVDHKPSNTKVDKRVESK |
| | | | YGPPCPPCPAPEFEGGPSVFLFPPKPKDTLMISRTPE |
| | | | VTCVVVDVSQEDPEVQFNWYVDGVEVHNAKTKPRE |
| | | | EQFNSTYRVVSVLTVLAQDWLNGKEYKCKVSNKGLP |
| | | | SSIEKTISKAKGQPREPQVYTLPPSQEEMTKNQVSLT |
| | | | CLVKGFYPSDIAVEWESNGQPENNYKTTPPVLDSDG |
| | | | SFFLYSRLTVDKSRWQEGNVFSCSVMHEALHNQYT |
| | | | QKSLSLSLGK |
| ANT4044- | Vk Light chain | 243 | DIQMTQSPSTLSASVGDRVTITCKASQDVGTAVDWY |
| ANT4044-A2 Vk | | | QQKPGQAPKLLIYWASTRHTGVPDRFSGSGSGTDFT |
| Light chain | | | LTISRLQPEDFAVYYCQQYNSYPLTFGQGTKVDIKRT |
| | | | VAAPSVFIFPPSDEQLKSGTASVVCLLNNFYPREAKV |
| | | | QWKVDNALQSGNSQESVTEQDSKDSTYSLSSTLTLS |
| | | | KADYEKHKVYACEVTHQGLSSPVTKSFNRGEC |
| Murine J591 | HCDR1 (protein) | 244 | GYTFTEYTIH |
| Variable Heavy | | | |
| chain | | | |
| Alternative J591 | Heavy chain | 245 | EVQLVQSGPEVKKPGATVKISCKTSGYTFTEYTIHWV |
| Variable Heavy | variable | | KQAPGKGLEWIGNINPNNGGTTYNQKFEDKATLTVD |
| chain | | | KSTDTAYMELSSLRSEDTAVYYCAAGWNFDYWGQG |
| | | | TLLTVSS |
| Alternative J591 | Light chain | 246 | DIQMTQSPSSLSTSVGDRVTLTCKASQDVGTAVDWY |
| Variable Light | variable | | QQKPGPSPKLLIYWASTRHTGIPSRFSGSGSGTDFTL |
| chain | sequence | | TISSLQPEDFADYYCQQYNSYPLTFGPGTKVDIK |

TABLE 2
Summary of amino acid and nucleotide sequences for CAIX-binding
antibodies for use according to the invention

| | | SEQ ID | |
|---|---|---|---|
| Antibody ID | Region | NO: | Amino acid or nucleotide sequence |
| Girentuximab | HCDR1 (protein) | 49 | NYYMS |
| chimeric VHO | HCDR2 (protein) | 50 | AINSDGGITYYLDTVKG |
| Variable Heavy | HCDR3 (protein) | 51 | HRSGYFSMDY |
| chain | VH (protein) | 52 | DVKLVESGGGLVKLGGSLKLSCAASGFTFSNYYMSWV |
| | | | RQTPEKRLELVAAINSDGGITYYLDTVKGRFTISRDNAK |
| | | | NTLYLQMSSLKSEDTALFYCARHRSGYFSMDYWGQG |
| | | | TSVTVSS |
| | HCDR1 (DNA) | 53 | AACTATTACATGTCT |
| | HCDR2 (DNA) | 54 | GCCATTAATAGTGATGGTGGTATCACCTACTATCTAG |
| | | | ACACTGTGAAGGGC |
| | HCDR3 (DNA) | 55 | CACCGCTCGGGCTACTTTTCTATGGACTAC |
| | VH (DNA) | 56 | GACGTGAAGCTCGTGGAGTCTGGGGGAGGCTTAGT |
| | | | GAAGCTTGGAGGATCCCTGAAACTCTCCTGTGCAGC |
| | | | CTCTGGATTCACTTTCAGTAACTATTACATGTCTTGG |
| | | | GTTCGCCAGACTCCAGAGAAGAGGCTGGAGTTGGT |
| | | | CGCAGCCATTAATAGTGATGGTGGTATCACCTACTA |
| | | | TCTAGACACTGTGAAGGGCCGATTCACCATTTCAAG |
| | | | AGACAATGCCAAGAACACCCTGTACCTGCAAATGAG |
| | | | CAGTCTGAAGTCTGAGGACACAGCCTTGTTTTACTG |
| | | | TGCAAGACACCGCTCGGGCTACTTTTCTATGGACTA |
| | | | CTGGGGTCAAGGAACCTCAGTCACCGTCTCCTCA |
| | HFR1 (protein) | 57 | DVKLVESGGGLVKLGGSLKLSCAASGFTFS |
| | HFR2 (protein) | 58 | WVRQTPEKRLELVA |
| | HFR3 (protein) | 59 | RFTISRDNAKNTLYLQMSSLKSEDTALFYCAR |
| | HFR4 (protein) | 60 | WGQGTSVTVSS |
| | HFR1 (DNA) | 61 | GACGTGAAGCTCGTGGAGTCTGGGGGAGGCTTAGT |
| | | | GAAGCTTGGAGGATCCCTGAAACTCTCCTGTGCAGC |
| | | | CTCTGGATTCACTTTCAGT |
| | HFR2 (DNA) | 62 | TGGGTTCGCCAGACTCCAGAGAAGAGGCTGGAGTT |
| | | | GGTCGCA |
| | HFR3 (DNA) | 63 | CGATTCACCATTTCAAGAGACAATGCCAAGAACACC |
| | | | CTGTACCTGCAAATGAGCAGTCTGAAGTCTGAGGAC |
| | | | ACAGCCTTGTTTTACTGTGCAAGA |
| | HFR4 (DNA) | 64 | TGGGGTCAAGGAACCTCAGTCACCGTCTCCTCA |
| Girentuximab | HCDR1 (protein) | 65 | NYYMS |
| humanised VH1 | HCDR2 (protein) | 66 | AINSDGGITYYLDTVKG |
| Variable Heavy | HCDR3 (protein) | 67 | HRSGYFSMDY |
| chain | VH (protein) | 68 | DVKLVESGGGLVKPGGSLRLSCAASGFTFSNYYMSW |
| | | | VRQAPGKGLELVAAINSDGGITYYLDTVKGRFTISRDN |
| | | | AKNTLYLQMSSLKSEDTALYYCARHRSGYFSMDYWG |
| | | | QGTSVTVSS |
| | HCDR1 (DNA) | 69 | AACTACTACATGAGC |
| | HCDR2 (DNA) | 70 | GCCATTAACAGTGACGGTGGCATCACCTACTACCTG |
| | | | GACACCGTGAAGGGC |
| | HCDR3 (DNA) | 71 | CACAGGAGCGGCTACTTCTCTATGGACTAC |
| | VH (DNA) | 72 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAGCAGCCTGAAGAGCGAGGACACGGCCCTGTATT |
| | | | ACTGTGCGAGACACAGGAGCGGCTACTTCTCTATGG |
| | | | ACTACTGGGGCCAGGGCACCAGCGTCACTGTCTCC |
| | | | TCA |
| | HFR1 (protein) | 73 | DVKLVESGGGLVKPGGSLRLSCAASGFTFS |
| | HFR2 (protein) | 74 | WVRQAPGKGLELVA |
| | HFR3 (protein) | 75 | RFTISRDNAKNTLYLQMSSLKSEDTALYYCAR |
| | HFR4 (protein) | 76 | WGQGTSVTVSS |
| | HFR1 (DNA) | 77 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 78 | TGGGTGCGCCAGGCTCCAGGGAAGGGGCTGGAGC |
| | | | TGGTTGCC |
| | HFR3 (DNA) | 79 | CGATTCACCATCTCCAGGGACAACGCCAAGAACACC |
| | | | CTGTATCTGCAAATGAGCAGCCTGAAGAGCGAGGA |
| | | | CACGGCCCTGTATTACTGTGCGAGA |
| | HFR4 (DNA) | 80 | TGGGGCCAGGGCACCAGCGTCACTGTCTCCTCA |
| Girentuximab | HCDR1 (protein) | 81 | NYYMS |
| humanised VH3 | HCDR2 (protein) | 82 | AINSDGGITYYLDTVKG |
| Variable Heavy | HCDR3 (protein) | 83 | HRSGYFSMDY |
| chain | VH (protein) | 84 | DVKLVESGGGLVKPGGSLRLSCAASGFTFSNYYMSW |
| | | | VRQAPGKGLELVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 85 | AACTACTACATGAGC |
| | HCDR2 (DNA) | 86 | CCATTAACAGTGACGGTGGCATCACCTACTACCTGG |
| | | | ACACCGTGAAGGGC |
| | HCDR3 (DNA) | 87 | CACAGGAGCGGCTACTTCTCTATGGACTAC |
| | VH (DNA) | 88 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 89 | DVKLVESGGGLVKPGGSLRLSCAASGFTFS |
| | HFR2 (protein) | 90 | WVRQAPGKGLELVA |
| | HFR3 (protein) | 91 | RFTISRDNAKNTLYLQMNSLRAEDTALYYCAR |
| | HFR4 (protein) | 92 | WGQGTLVTVSS |
| | HFR1 (DNA) | 93 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 94 | TGGGTGCGCCAGGCTCCAGGGAAGGGGCTGGAGC |
| | | | TGGTTGCC |
| | HFR3 (DNA) | 95 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 96 | TGGGGCCAGGGCACCCTGGTCACTGTCTCCTCA |
| Girentuximab | HCDR1 (protein) | 97 | NYYMS |
| humanised VH4 | HCDR2 (protein) | 98 | AINSDGGITYYLDTVKG |
| Variable Heavy | HCDR3 (protein) | 99 | HRSGYFSMDY |
| chain | VH (protein) | 100 | EVQLVESGGGLVKPGGSLRLSCAASGFTFSNYYMSW |
| | | | VRQAPGKGLELVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 101 | AACTACTACATGAGC |
| | HCDR2 (DNA) | 102 | GCCATTAACAGTGACGGTGGCATCACCTACTACCTG |
| | | | GACACCGTGAAGGGC |
| | HCDR3 (DNA) | 103 | CACAGGAGCGGCTACTTCTCTATGGACTAC |
| | VH (DNA) | 104 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 105 | EVQLVESGGGLVKPGGSLRLSCAASGFTFS |
| | HFR2 (protein) | 106 | WVRQAPGKGLELVA |
| | HFR3 (protein) | 107 | RFTISRDNAKNTLYLQMNSLRAEDTALYYCAR |
| | HFR4 (protein) | 108 | WGQGTLVTVSS |
| | HFR1 (DNA) | 109 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 110 | TGGGTGCGCCAGGCTCCAGGGAAGGGGCTGGAGC |
| | | | TGGTTGCC |
| | HFR3 (DNA) | 111 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 112 | TGGGGCCAGGGCACCCTGGTCACTGTCTCCTCA |
| Girentuximab | HCDR1 (protein) | 113 | NYYMS |
| humanised VH5 | HCDR2 (protein) | 114 | AINSDGGITYYLDTVKG |
| Variable Heavy | HCDR3 (protein) | 115 | HRSGYFSMDY |
| chain | VH (protein) | 116 | EVQLVESGGGLVKPGGSLRLSCAASGFTFSNYYMSW |
| | | | VRQAPGKGLEWVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 117 | AACTACTACATGAGC |
| | HCDR2 (DNA) | 118 | GCCATTAACAGTGACGGTGGCATCACCTACTACCTG |
| | | | GACACCGTGAAGGGC |
| | HCDR3 (DNA) | 119 | CACAGGAGCGGCTACTTCTCTATGGACTAC |
| | VH (DNA) | 120 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGTG |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 121 | EVQLVESGGGLVKPGGSLRLSCAASGFTFS |
| | HFR2 (protein) | 122 | WVRQAPGKGLEWVA |
| | HFR3 (protein) | 123 | RFTISRDNAKNTLYLQMNSLRAEDTALYYCAR |
| | HFR4 (protein) | 124 | WGQGTLVTVSS |
| | HFR1 (DNA) | 125 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 126 | TGGGTGCGCCAGGCTCCAGGGAAGGGGCTGGAGT |
| | | | GGGTTGCC |
| | HFR3 (DNA) | 127 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 128 | TGGGGCCAGGGCACCCTGGTCACTGTCTCCTCA |
| Girentuximab | LCDR1 (protein) | 129 | KASQNVVSAVA |
| Chimeric Vk0 | LCDR2 | 130 | SASNRYT |
| Variable light | (protein) | | |
| chain | LCDR3 (protein) | 131 | QQYSNYPWT |
| | VL (protein) | 132 | DIVMTQSQRFMSTTVGDRVSITCKASQNVVSAVAWYQ |
| | | | QKPGQSPKLLIYSASNRYTGVPDRFTGSGSGTDFTLTI |
| | | | SNMQSEDLADFFCQQYSNYPWTFGGGTKLEIK |
| | LCDR1 (DNA) | 133 | AAGGCCAGTCAGAATGTGGTTTCTGCTGTTGCC |
| | LCDR2 (DNA) | 134 | TCAGCATCCAATCGGTACACT |
| | LCDR3 (DNA) | 135 | CAACAATATAGCAACTATCCGTGGACG |
| | VL (DNA) | 136 | GACATTGTGATGACCCAGTCTCAAAGATTCATGTCC |
| | | | ACAACAGTAGGAGACAGGGTCAGCATCACCTGCAA |
| | | | GGCCAGTCAGAATGTGGTTTCTGCTGTTGCCTGGTA |
| | | | TCAACAGAAACCAGGACAATCTCCTAAACTACTGATT |
| | | | TACTCAGCATCCAATCGGTACACTGGAGTCCCTGAT |
| | | | CGCTTCACAGGCAGTGGATCTGGGACAGATTTCACT |
| | | | CTCACCATTAGCAATATGCAGTCTGAAGACCTGGCT |
| | | | GATTTTTTCTGTCAACAATATAGCAACTATCCGTGGA |
| | | | CGTTCGGTGGAGGCACCAAGCTGGAAATCAAA |
| | LFR1 | 137 | DIVMTQSQRFMSTTVGDRVSITC |
| | (protein) | | |
| | LFR2 (protein) | 138 | WYQQKPGQSPKLLIY |
| | LFR3 (protein) | 139 | GVPDRFTGSGSGTDFTLTISNMQSEDLADFFC |
| | LFR4 | 140 | FGGGTKLEIK |
| | (protein) | | |
| | LFR1 | 141 | GACATTGTGATGACCCAGTCTCAAAGATTCATGTCC |
| | (DNA) | | ACAACAGTAGGAGACAGGGTCAGCATCACCTGC |
| | LFR2 (DNA) | 142 | TGGTATCAACAGAAACCAGGACAATCTCCTAAACTA |
| | | | CTGATTTAC |
| | LFR3 (DNA) | 143 | GGAGTCCCTGATCGCTTCACAGGCAGTGGATCTGG |
| | | | GACAGATTTCACTCTCACCATTAGCAATATGCAGTCT |
| | | | GAAGACCTGGCTGATTTTTTCTGT |
| | LFR4 (DNA) | 144 | TTCGGTGGAGGCACCAAGCTGGAAATCAAA |
| Girentuximab | LCDR1 (protein) | 145 | KASQNVVSAVA |
| Humanised Vk1 | LCDR2 | 146 | SASNRYT |
| Variable light | (protein) | | |
| chain | LCDR3 (protein) | 147 | QQYSNYPWT |
| | VL (protein) | 148 | DIVMTQSPSFLSASVGDRVTITCKASQNVVSAVAWYQ |
| | | | QKPGQAPKLLIYSASNRYTGVPDRFTGSGSGTDFTLTI |
| | | | SSLQSEDLADYFCQQYSNYPWTFGGGTKVEIK |
| | LCDR1 (DNA) | 149 | AAGGCAAGTCAGAACGTGGTGAGTGCTGTGGCC |
| | LCDR2 (DNA) | 150 | AGCGCCTCCAACAGGTACACC |
| | LCDR3 (DNA) | 151 | CAACAGTACAGCAATTACCCTTGGACG |
| | VL (DNA) | 152 | GACATCGTGATGACCCAGTCTCCATCCTTCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAAGCTCCTGAT |
| | | | CTATAGCGCCTCCAACAGGTACACCGGGGTCCCAG |
| | | | ACAGGTTCACCGGCAGTGGATCTGGGACAGATTTCA |
| | | | CTCTCACCATCAGCAGCCTGCAGAGCGAAGATCTG |
| | | | GCAGACTATTTCTGTCAACAGTACAGCAATTACCCTT |
| | | | GGACGTTCGGCGGCGGGACCAAGGTGGAAATCAAA |
| | LFR1 | 153 | DIVMTQSPSFLSASVGDRVTITC |
| | (protein) | | |
| | LFR2 (protein) | 154 | WYQQKPGQAPKLLIY |
| | LFR3 (protein) | 155 | GVPDRFTGSGSGTDFTLTISSLQSEDLADYFC |
| | LFR4 | 156 | FGGGTKVEIK |
| | (protein) | | |
| | LFR1 | 157 | GACATCGTGATGACCCAGTCTCCATCCTTCCTGTCT |
| | (DNA) | | GCATCTGTAGGAGACAGAGTCACCATCACTTGC |
| | LFR2 (DNA) | 158 | TGGTATCAGCAGAAACCAGGGCAGGCTCCTAAGCT |
| | | | CCTGATCTAT |
| | LFR3 (DNA) | 159 | GGGGTCCCAGACAGGTTCACCGGCAGTGGATCTGG |
| | | | GACAGATTTCACTCTCACCATCAGCAGCCTGCAGAG |
| | | | CGAAGATCTGGCAGACTATTTCTGT |
| | LFR4 (DNA) | 160 | TTCGGCGGCGGGACCAAGGTGGAAATCAAA |
| Girentuximab | LCDR1 (protein) | 161 | KASQNVVSAVA |
| Humanised Vk2 | LCDR2 | 162 | SASNRYT |
| Variable light | (protein) | | |
| chain | LCDR3 (protein) | 163 | QQYSNYPWT |
| | VL (protein) | 164 | DIVMTQSPSSLSASVGDRVTITCKASQNVVSAVAWYQ |
| | | | QKPGQAPRLLIYSASNRYTGVPDRFTGSGSGTDFTLTI |
| | | | SSLQAEDLADYFCQQYSNYPWTFGGGTKVEIK |
| | LCDR1 (DNA) | 165 | AAGGCAAGTCAGAACGTGGTGAGTGCTGTGGCC |
| | LCDR2 (DNA) | 166 | AGCGCCTCCAACAGGTACACC |
| | LCDR3 (DNA) | 167 | CAACAGTACAGCAATTACCCTTGGACG |
| | VL (DNA) | 168 | GACATCGTGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCACCGGCAGTGGATCTGGGACAGATTT |
| | | | CACTCTCACCATCAGCAGCCTGCAGGCCGAAGATCT |
| | | | GGCAGACTATTTCTGTCAACAGTACAGCAATTACCC |
| | | | TTGGACGTTCGGCGGGGGGACCAAGGTGGAAATCA |
| | | | AA |
| | LFR1 | 169 | DIVMTQSPSSLSASVGDRVTITC |
| | (protein) | | |
| | LFR2 (protein) | 170 | WYQQKPGQAPRLLIY |
| | LFR3 (protein) | 171 | GVPDRFTGSGSGTDFTLTISSLQAEDLADYFC |
| | LFR4 | 172 | FGGGTKVEIK |
| | (protein) | | |
| | LFR1 | 173 | GACATCGTGATGACCCAGTCTCCATCCAGCCTGTCT |
| | (DNA) | | GCATCTGTAGGAGACAGAGTCACCATCACTTGC |
| | LFR2 (DNA) | 174 | TGGTATCAGCAGAAACCAGGGCAGGCTCCTAGGCT |
| | | | CCTGATCTAT |
| | LFR3 (DNA) | 175 | GGGGTCCCAGACAGGTTCACCGGCAGTGGATCTGG |
| | | | GACAGATTTCACTCTCACCATCAGCAGCCTGCAGGC |
| | | | CGAAGATCTGGCAGACTATTTCTGT |
| | LFR4 (DNA) | 176 | TTCGGCGGGGGACCAAGGTGGAAATCAAA |
| Girentuximab | LCDR1 (protein) | 177 | KASQNVVSAVA |
| Humanised Vk3 | LCDR2 | 178 | SASNRYT |
| Variable light | (protein) | | |
| chain | LCDR3 (protein) | 179 | QQYSNYPWT |
| | VL (protein) | 180 | DIQMTQSPSSLSASVGDRVTITCKASQNVVSAVAWYQ |
| | | | QKPGQAPRLLIYSASNRYTGVPDRFSGSGSGTDFTLTI |
| | | | SSLQAEDLADYFCQQYSNYPWTFGGGTKVEIK |
| | LCDR1 (DNA) | 181 | AAGGCAAGTCAGAACGTGGTGAGTGCTGTGGCC |
| | LCDR2 (DNA) | 182 | AGCGCCTCCAACAGGTACACC |
| | LCDR3 (DNA) | 183 | CAACAGTACAGCAATTACCCTTGGACG |
| | VL (DNA) | 184 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTTCTGTCAACAGTACAGCAATTACCC |
| | | | TTGGACGTTCGGCGGCGGGACCAAGGTGGAAATCA |
| | | | AA |
| | LFR1 | 185 | DIQMTQSPSSLSASVGDRVTITC |
| | (protein) | | |
| | LFR2 (protein) | 186 | WYQQKPGQAPRLLIY |
| | LFR3 (protein) | 187 | GVPDRFSGSGSGTDFTLTISSLQAEDLADYFC |
| | LFR4 | 188 | FGGGTKVEIK |
| | (protein) | | |
| | LFR1 | 189 | GACATCCAGATGACCCAGTCTCCATCCAGCCTGTCT |
| | (DNA) | | GCATCTGTAGGAGACAGAGTCACCATCACTTGC |
| | LFR2 (DNA) | 190 | TGGTATCAGCAGAAACCAGGGCAGGCTCCTAGGCT |
| | | | CCTGATCTAT |
| | LFR3 (DNA) | 191 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTTCTGT |
| | LFR4 (DNA) | 192 | TTCGGCGGGGGACCAAGGTGGAAATCAAA |
| Girentuximab | LCDR1 (protein) | 193 | KASQNVVSAVA |
| Humanised Vk4 | LCDR2 | 194 | SASNRYT |
| Variable light | (protein) | | |
| chain | LCDR3 (protein) | 195 | QQYSNYPWT |
| | VL (protein) | 196 | DIQMTQSPSSLSASVGDRVTITCKASQNVVSAVAWYQ |
| | | | QKPGQAPRLLIYSASNRYTGVPDRFSGSGSGTDFTLTI |
| | | | SSLQAEDLADYYCQQYSNYPWTFGGGTKVEIK |
| | LCDR1 (DNA) | 197 | AAGGCAAGTCAGAACGTGGTGAGTGCTGTGGCC |
| | LCDR2 (DNA) | 198 | AGCGCCTCCAACAGGTACACC |
| | LCDR3 (DNA) | 199 | CAACAGTACAGCAATTACCCTTGGACG |
| | VL (DNA) | 200 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTACTGTCAACAGTACAGCAATTACCC |
| | | | TTGGACGTTCGGCGGCGGGACCAAGGTGGAAATCA |
| | | | AA |
| | LFR1 | 201 | DIQMTQSPSSLSASVGDRVTITC |
| | (protein) | | |
| | LFR2 (protein) | 202 | WYQQKPGQAPRLLIY |
| | LFR3 (protein) | 203 | GVPDRFSGSGSGTDFTLTISSLQAEDLADYYC |
| | LFR4 | 204 | FGGGTKVEIK |
| | (protein) | | |
| | LFR1 | 205 | GACATCCAGATGACCCAGTCTCCATCCAGCCTGTCT |
| | (DNA) | | GCATCTGTAGGAGACAGAGTCACCATCACTTGC |
| | LFR2 (DNA) | 206 | TGGTATCAGCAGAAACCAGGGCAGGCTCCTAGGCT |
| | | | CCTGATCTAT |
| | LFR3 (DNA) | 207 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTACTGT |
| | LFR4 (DNA) | 208 | TTCGGCGGCGGGACCAAGGTGGAAATCAAA |
| Girentuximab | LCDR1 (protein) | 209 | KASQNVVSAVA |
| Humanised Vk5 | LCDR2 | 210 | SASNRYT |
| Variable light | (protein) | | |
| chain | LCDR3 (protein) | 211 | QQYSNYPWT |
| | VL (protein) | 212 | DIQMTQSPSSLSASVGDRVTITCKASQNVVSAVAWYQ |
| | | | QKPGQAPRRLIYSASNRYTGVPDRFSGSGSGTDFTLTI |
| | | | SSLQAEDLADYYCQQYSNYPWTFGGGTKVEIK |
| | LCDR1 (DNA) | 213 | AAGGCAAGTCAGAACGTGGTGAGTGCTGTGGCC |
| | LCDR2 (DNA) | 214 | AGCGCCTCCAACAGGTACACC |
| | LCDR3 (DNA) | 215 | CAACAGTACAGCAATTACCCTTGGACG |
| | VL (DNA) | 216 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGAGGCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTACTGTCAACAGTACAGCAATTACCC |
| | | | TTGGACGTTCGGCGGGGGGACCAAGGTGGAAATCA |
| | | | AA |
| | LFR1 | 217 | DIQMTQSPSSLSASVGDRVTITC |
| | (protein) | | |
| | LFR2 (protein) | 218 | WYQQKPGQAPRRLIY |
| | LFR3 (protein) | 219 | GVPDRFSGSGSGTDFTLTISSLQAEDLADYYC |
| | LFR4 | 220 | FGGGTKVEIK |
| | (protein) | | |
| | LFR1 | 22 | GACATCCAGATGACCCAGTCTCCATCCAGCCTGTCT |
| | (DNA) | | GCATCTGTAGGAGACAGAGTCACCATCACTTGC |
| | LFR2 (DNA) | 222 | TGGTATCAGCAGAAACCAGGGCAGGCTCCTAGGAG |
| | | | GCTGATCTAT |
| | LFR3 (DNA) | 223 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTACTGT |
| | LFR4 (DNA) | 224 | TTCGGCGGCGGGACCAAGGTGGAAATCAAA |
| Girentuximab | IgG1 HC | 225 | ASTKGPSVFPLAPSSKSTSGGTAALGCLVKDYFPEPVT |
| unmodified | (protein) | | VSWNSGALTSGVHTFPAVLQSSGLYSLSSVVTVPSSS |
| human IgG1 | | | LGTQTYICNVNHKPSNTKVDKKVEPKSCDKTHTCPPC |
| constant chain | | | PAPELLGGPSVFLFPPKPKDTLMISRTPEVTCVVVDVS |
| region | | | HEDPEVKFNWYVDGVEVHNAKTKPREEQYNSTYRVV |
| | | | SVLTVLHQDWLNGKEYKCKVSNKALPAPIEKTISKAKG |
| | | | QPREPQVYTLPPSRDELTKNQVSLTCLVKGFYPSDIAV |
| | | | EWESNGQPENNYKTTPPVLDSDGSFFLYSKLTVDKSR |
| | | | WQQGNVFSCSVMHEALHNHYTQKSLSLSPGK |
| Girentuximab | IgG1 H310A | 226 | ASTKGPSVFPLAPSSKSTSGGTAALGCLVKDYFPEPVT |
| modified human | H435Q HC | | VSWNSGALTSGVHTFPAVLQSSGLYSLSSVVTVPSSS |
| IgG1 constant | (protein) | | LGTQTYICNVNHKPSNTKVDKKVEPKSCDKTHTCPPC |
| chain region | | | PAPELLGGPSVFLFPPKPKDTLMISRTPEVTCVVVDVS |
| | | | HEDPEVKFNWYVDGVEVHNAKTKPREEQYNSTYRVV |
| | | | SVLTVLAQDWLNGKEYKCKVSNKALPAPIEKTISKAKG |
| | | | QPREPQVYTLPPSRDELTKNQVSLTCLVKGFYPSDIAV |
| | | | EWESNGQPENNYKTTPPVLDSDGSFFLYSKLTVDKSR |
| | | | WQQGNVFSCSVMHEALHNQYTQKSLSLSPGK |
| Girentuximab | IgG4 S228P | 227 | ASTKGPSVFPLAPCSRSTSESTAALGCLVKDYFPEPVT |
| modified human | L235E HC | | VSWNSGALTSGVHTFPAVLQSSGLYSLSSVVTVPSSS |
| IgG4 constant | (protein) | | LGTKTYTCNVDHKPSNTKVDKRVESKYGPPCPPCPAP |
| chain region | | | EFEGGPSVFLFPPKPKDTLMISRTPEVTCVVVDVSQED |
| | | | PEVQFNWYVDGVEVHNAKTKPREEQFNSTYRVVSVLT |
| | | | VLHQDWLNGKEYKCKVSNKGLPSSIEKTISKAKGQPR |
| | | | EPQVYTLPPSQEEMTKNQVSLTCLVKGFYPSDIAVEW |
| | | | ESNGQPENNYKTTPPVLDSDGSFFLYSRLTVDKSRWQ |
| | | | EGNVFSCSVMHEALHNHYTQKSLSLSLGK |
| Girentuximab | IgG4 S228P | 228 | ASTKGPSVFPLAPCSRSTSESTAALGCLVKDYFPEPVT |
| modified human | L235E H310A | | VSWNSGALTSGVHTFPAVLQSSGLYSLSSVVTVPSSS |
| IgG4 constant | H435Q HC | | LGTKTYTCNVDHKPSNTKVDKRVESKYGPPCPPCPAP |
| chain region | (protein) | | EFEGGPSVFLFPPKPKDTLMISRTPEVTCVVVDVSQED |
| | | | PEVQFNWYVDGVEVHNAKTKPREEQFNSTYRVVSVLT |
| | | | VLAQDWLNGKEYKCKVSNKGLPSSIEKTISKAKGQPRE |
| | | | PQVYTLPPSQEEMTKNQVSLTCLVKGFYPSDIAVEWE |
| | | | SNGQPENNYKTTPPVLDSDGSFFLYSRLTVDKSRWQE |
| | | | GNVFSCSVMHEALHNQYTQKSLSLSLGK |
| Girentuximab | Human κ LC | 229 | RTVAAPSVFIFPPSDEQLKSGTASVVCLLNNFYPREAK |
| kappa light chain | constant region | | VQWKVDNALQSGNSQESVTEQDSKDSTYSLSSTLTLS |
| constant region | | | KADYEKHKVYACEVTHQGLSSPVTKSFNRGEC |
| Girentuximab | IgG1 VH4 heavy | 230 | EVQLVESGGGLVKPGGSLRLSCAASGFTFSNYYMSW |
| native IgG1 | chain | | VRQAPGKGLELVAAINSDGGITYYLDTVKGRFTISRDN |
| allotype Gm | | | AKNTLYLQMNSLRAEDTALYYCARHRSGYFSMDYWG |
| (1,17) VH4 | | | QGTLVTVSSASTKGPSVFPLAPSSKSTSGGTAALGCLV |
| heavy chain | | | KDYFPEPVTVSWNSGALTSGVHTFPAVLQSSGLYSLS |
| | | | SVVTVPSSSLGTQTYICNVNHKPSNTKVDKKVEPKSCD |
| | | | KTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLHQDWLNGKEYKCKVSNKALPAPIE |
| | | | KTISKAKGQPREPQVYTLPPSRDELTKNQVSLTCLVKG |
| | | | FYPSDIAVEWESNGQPENNYKTTPPVLDSDGSFFLYS |
| | | | KLTVDKSRWQQGNVFSCSVMHEALHNHYTQKSLSLS |
| | | | PGK |
| hGirentuximab | FcRn-null IgG1 | 231 | EVQLVESGGGLVKPGGSLRLSCAASGFTFSNYYMSW |
| IgG1 allotype | allotype H130A | | VRQAPGKGLELVAAINSDGGITYYLDTVKGRFTISRDN |
| Gm (1,17) | H435Q | | AKNTLYLQMNSLRAEDTALYYCARHRSGYFSMDYWG |
| RADmAb VH4 | | | QGTLVTVSSASTKGPSVFPLAPSSKSTSGGTAALGCLV |
| heavy chain | | | KDYFPEPVTVSWNSGALTSGVHTFPAVLQSSGLYSLS |
| | | | SVVTVPSSSLGTQTYICNVNHKPSNTKVDKKVEPKSCD |
| | | | KTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLAQDWLNGKEYKCKVSNKALPAPIE |
| | | | KTISKAKGQPREPQVYTLPPSRDELTKNQVSLTCLVKG |
| | | | FYPSDIAVEWESNGQPENNYKTTPPVLDSDGSFFLYS |
| | | | KLTVDKSRWQQGNVFSCSVMHEALHNQYTQKSLSLS |
| | | | PGK |
| hGirentuximab | FcRγ-null IgG4 | 232 | EVQLVESGGGLVKPGGSLRLSCAASGFTFSNYYMSW |
| IgG4 RADmAb | S228P L235E | | VRQAPGKGLELVAAINSDGGITYYLDTVKGRFTISRDN |
| VH4 heavy | | | AKNTLYLQMNSLRAEDTALYYCARHRSGYFSMDYWG |
| chain | | | QGTLVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFEGGPSVFLFPPKPKDTLMISRTPEVTC |
| | | | VVVDVSQEDPEVQFNWYVDGVEVHNAKTKPREEQFN |
| | | | STYRVVSVLTVLHQDWLNGKEYKCKVSNKGLPSSIEKT |
| | | | ISKAKGQPREPQVYTLPPSQEEMTKNQVSLTCLVKGF |
| | | | YPSDIAVEWESNGQPENNYKTTPPVLDSDGSFFLYSR |
| | | | LTVDKSRWQEGNVFSCSVMHEALHNHYTQKSLSLSLG |
| | | | K |
| hGirentuximab | FcRn + FcRγ-null | 233 | EVQLVESGGGLVKPGGSLRLSCAASGFTFSNYYMSW |
| IgG4 RADmAb | IgG4 S228P | | VRQAPGKGLELVAAINSDGGITYYLDTVKGRFTISRDN |
| VH4 heavy | L235E H310A | | AKNTLYLQMNSLRAEDTALYYCARHRSGYFSMDYWG |
| chain | H435Q | | QGTLVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFEGGPSVFLFPPKPKDTLMISRTPEVTC |
| | | | VVVDVSQEDPEVQFNWYVDGVEVHNAKTKPREEQFN |
| | | | STYRVVSVLTVLAQDWLNGKEYKCKVSNKGLPSSIEKT |
| | | | ISKAKGQPREPQVYTLPPSQEEMTKNQVSLTCLVKGF |
| | | | YPSDIAVEWESNGQPENNYKTTPPVLDSDGSFFLYSR |
| | | | LTVDKSRWQEGNVFSCSVMHEALHNQYTQKSLSLSL |
| | | | GK |
| hGirentuximab | Vκ Light Chain | 234 | DIQMTQSPSSLSASVGDRVTITCKASQNVVSAVAWYQ |
| Vκ Light Chain | | | QKPGQAPRLLIYSASNRYTGVPDRFSGSGSGTDFTLTI |
| | | | SSLQAEDLADYYCQQYSNYPWTFGGGTKVEIKRTVAA |
| | | | PSVFIFPPSDEQLKSGTASVVCLLNNFYPREAKVQWKV |
| | | | DNALQSGNSQESVTEQDSKDSTYSLSSTLTLSKADYE |
| | | | KHKVYACEVTHQGLSSPVTKSFNRGEC |

### EXAMPLES

### Example 1: In Vivo Efficacy of ¹⁷⁷Lu-Anti-CAIX Antibody+DNA-PKi in Metastatic Renal Cell Carcinoma Xenograft Model

Female BALB/c nude mice with established SK-RC-52 xenografts (approx. 100 mm³) were used for this study. Mice were split into one of the following 3 groups:
Vehicle control (oral) n=4;
¹⁷⁷Lu-anti-CAIX antibody only (DOTA-GmAb, as described herein; 162 μCi/6 MBq) n=4;
50 mg/kg M3814 DNA-PKi orally dosed and ¹⁷⁷Lu-anti-CAIX antibody (162 μCi/6 MBq) n=4.

Animals were treated as follows:
Day −14: injection of 60 female mice with RK-RC-52 metastatic RCC cells in matrigel;
Day 0: ¹⁷⁷Lu-anti-CAIX antibody i.v. administration, single dose once only
Day 1-7: daily oral dosing of vehicle/M3814 @ 50 mg/kg.

SPECT/MRI imaging was conducted at days 3 and 6 to determine biodistribution.

Tumour growth curves were monitored in mice for up to 6 months or ethical limits.

FIG. 1 shows the results of ¹⁷⁷Lu-anti-CAIX antibody SPECT imaging in the mice. A single administration of ¹⁷⁷Lu-anti-CAIX antibody specifically delivers cytotoxic radiation to the tumour over a prolonged period.

FIG. 2 shows tumour volume (mm³) and percentage change in tumour volume over >143 days. Mice treated with ¹⁷⁷Lu-anti-CAIX antibody and M3814 had significantly lower tumour size after 143 days compared to mice treated with antibody alone.

All mice that received combination treatment with ¹⁷⁷Lu-anti-CAIX antibody and M3814 were in complete remission at the conclusion of the study:

| | Progressive | Complete | |
|---|---|---|---|
| Treatment Group | Disease | Remission | Total |
| Vehicle | 4 | — | 4 |
| ¹⁷⁷Lu-anti-CAIX antibody | 3 (75%) | 1 (25%) | 4 |
| 6 MBq | | | |
| ¹⁷⁷Lu-anti-CAIX antibody | 0 | 4 (100%) | 4 |
| 6 MBq + M3814 | | | |
| ¹⁷⁷Lu-anti-CAIX antibody | 2 (50%) | 2 (50%) | 4 |
| 3 MBq + M3814 | | | |

### Example 2: In Vivo Efficacy of ¹⁷⁷Lu-Anti-PSMA Antibody+DNA-PKi in PSMA^{high} Prostate Cancer Xenoaraft Model

Male BALB/c nude mice (age 5-6 weeks) with established LNCaP xenografts (approx. 200 mm³) were used for this study. Mice were split into one of the following 3 groups:
Vehicle control (oral) n=3;
¹⁷⁷Lu-anti-PSMA antibody only (DOTA-HuJ501, as described herein; 162 μCi/6 MBq) n=4;
50 mg/kg M3814 DNA-PKi orally dosed and ¹⁷⁷Lu-anti-PSMA antibody (162 μCi/6 MBq) n=4.

Animals were treated as follows:
Day −20: injection of male mice with 5×10⁶ LNCaP cells;
Day 0: ¹⁷⁷Lu-anti-PSMA antibody i.v. administration, single dose once only
Day 1-14: daily oral dosing of vehicle/M3814 @ 50 mg/kg.

SPECT/MRI imaging was conducted at days 3 and 7 to determine biodistribution.

Tumour growth curves were monitored in mice for up to 6 months or ethical limits.

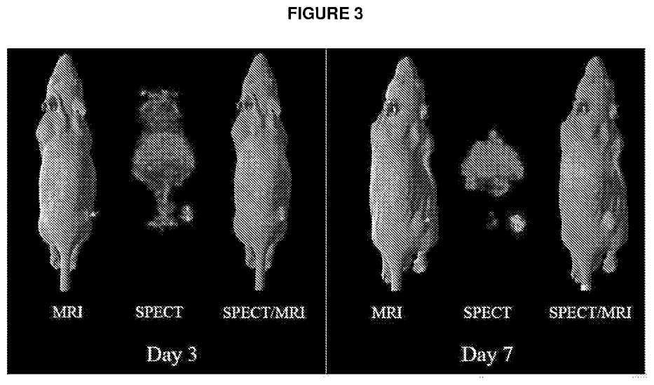

FIG. 3 shows the results of ¹⁷⁷Lu-anti-PSMA antibody SPECT imaging in the mice. Mice treated with ¹⁷⁷Lu-anti-PSMA antibody and M3814 had significantly smaller tumours 112 days after the commencement of treatment compared to mice treated with antibody alone.

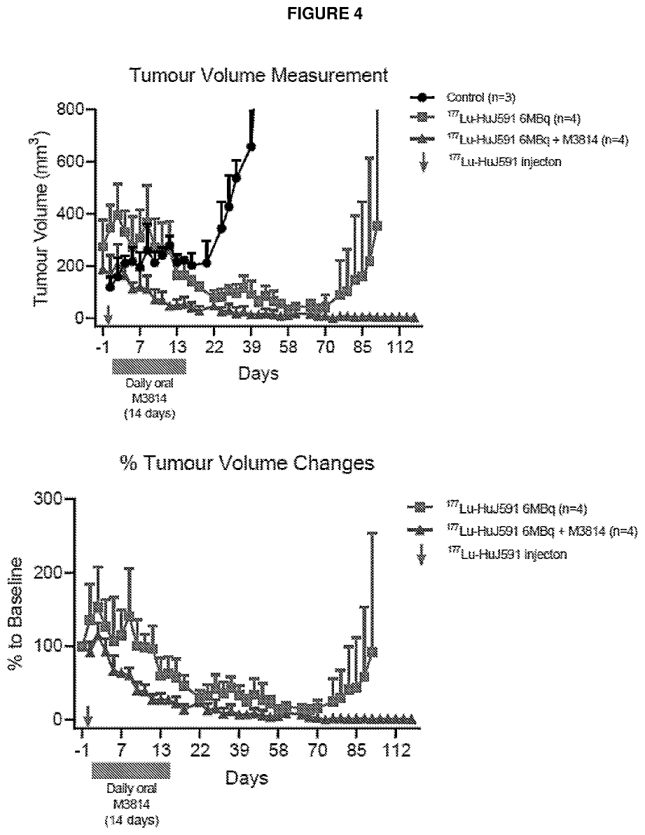

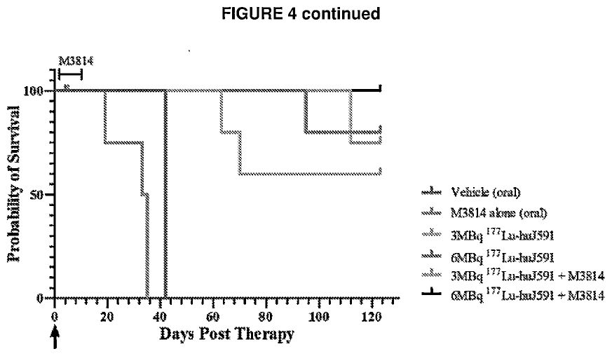

FIG. 4 shows tumour volume (mm³) and percentage change in tumour volume over >110 days.

A significant proportion of mice that received combination treatment with ¹⁷⁷Lu-anti-PSMA antibody and M3814 were in complete remission at the conclusion of the study and the results further indicated evidence of continued remission:

| | Progressive | Complete | Continued | |
|---|---|---|---|---|
| Treatment Group | Disease | Remission | Remission | Total |
| Vehicle | 3 | — | — | 3 |
| ¹⁷⁷Lu-anti-PSMA antibody | 3 (75%) | 1 (25%) | — | 4 |
| 6 MBq | | | | |
| ¹⁷⁷Lu-anti-PSMA antibody | 0 | 3 (75%) | 1 (25%) | 4 |
| 6 MBq + M3814 | | | | |

### Example 3: Clinical Trial of the Combination of ¹⁷⁷Lu-Qirentuximab and Peposertib (M3814) with CAIX-Expressing Renal Tumours

### Objectives

### Primary Objectives:

To determine the Maximum Tolerated Dose (MTD) of ¹⁷⁷Lu-girentuximab in combination with peposertib
Treatment emergent adverse events (TEAE): Type according to MedDRA (Medical Dictionary for Regulatory Activities), frequency, severity according to NCI CTCAE V5.0, seriousness, and relationship of study treatment will be assessed. Laboratory abnormalities will be assessed according to the NCI CTCAE v.5.0Events.

### Secondary Objectives:

Overall response rate of ¹⁷⁷Lu-girentuximab in combination peposertib as per RECISTv1.1
Estimate progression-free survival (PFS),
Estimate overall survival (OS),
Estimate duration of response
Assess safety and tolerability of combination therapy
Quality of life as determined using the EORTC QLQ-C30 questionnaire.

### Exploratory Objectives:

Correlation of PFS with CAIX expression
Correlation of OS with CAIX expression
Assess radiographic response using Zirconium-89 (⁸⁹Zr)-girentuximab by PET/CT.
Assess the distribution, lesion uptake, and dosimetry assessment of ¹⁷⁷Lu-labeled-girentuximab following administration using whole body planar and spect imaging and correlate results with patient outcome (e.g., response, PFS).

### Methodology:

### Study Design Phase I:

An open label, single-arm, randomized, parallel-group, multicenter dose finding study is conducted to evaluate ascending radioactive dose levels of ¹⁷⁷Lu-girentuximab administered intravenously in combination with peposertib in patients with relapsed/refractory CAIX-expressing renal tumours.

The protocol starts with a safety-lead in phase using a 3+3 design to establish the maximal tolerated dose (MTD) of ¹⁷⁷Lu-labeled-girentuximab in combination with up to 400 mg BID daily of peposertib from Day 1 until progression as determined by PET imaging. The initial starting dose of ¹⁷⁷Lu-labeled-girentuximab is 1110 MBq/m² which is <50% of the single agent dose established in prior studies and will proceed as shown in the schema below. Once the MTD is established, a Simon two-stage optimal design commences.

10 patients are enrolled in the first stage and if no responses are observed, the study is terminated. Patients treated at the MTD in the safety lead-in phase are included in the phase II. Total accrual across the safety-lead in and Phase II is 60 patients.

| | |
|---|---|
| Dose level −1 | 1110 MBq/m² + 300 mg peposertib |
| Dose level 1 | 1110 MBq/m² + 400 mg peposertib |
| Dose level 2 | 1480 MBq/m² + 400 mg peposertib |
| Dose level 3 | 1850 MBq/m² + 400 mg peposertib |

Alternatively, ¹⁷⁷Lu-labeled-girentuximab and peposertib are administered according to the following doses, wherein ¹⁷⁷Lu-labeled-girentuximab is administered in 3 doses, 3 weeks apart:

| | |
|---|---|
| Dose level −1 | 1110 MBq/m² + 100 mg peposertib |
| Dose level 1 | 1110 MBq/m² + 175 mg peposertib |
| Dose level 2 | 1480 MBq/m² + 250 mg peposertib |
| Dose level 3 | 1480 MBq/m² + one step lower than no DLT |
| | dose peposertib |
| Dose level 4 | 1480 MBq/m² + no DLT dose peposertib |
| Dose level 5 | 1850 MBq/m² + one step lower than no DLT |
| | dose peposertib |
| Dose level 6 | 1480 MBq/m² + no DLT dose peposertib |

All patients receive FDG PET Scans, CT or MRI as standard of care imaging. These will be used for assessment of extent of disease.

All patients undergo a ⁸⁹Zr-girentuximab PET/CT scan prior to every ¹⁷⁷Lu-girentuximab administration and ¹⁷⁷Lu whole body (WB) planar and SPECT/CT scans will be performed after each administration of ¹⁷⁷Lu-girentuximab. To be eligible for the study, the baseline ⁸⁹Zr-girentuximab PET/CT must be positive (show ⁸⁹Zr uptake within at least one metastatic lesion). However, ⁸⁹Zr-girentuximab PET/CT positivity is not required prior to doses two and three of ¹⁷⁷Lu-girentuximab. The first 10 patients treated at the MTD who agree to additional imaging receive three whole body planar scans (at 0-4 h, 48-72 h±6 h and 96-144 h±6 h) and a SPECT/CT scan at 48-72 h (±6 h) after the first dose administration of 177Lu-girentuximab.

For subsequent doses (2 and 3) of ¹⁷⁷Lu-girentuximab in the first 10 patients, and all doses of ¹⁷⁷Lu-girentuximab for the remainder of the patients, a single WB planar scan and single SPECT/CT are performed at 48-72 hours (+/−6 h) after each dose administration of ¹⁷⁷Lu-girentuximab. This is summarized in Table 1 below.

TABLE 1
Experimental Imaging

| | First 10 patients¹ | All remaining patients |
|---|---|---|
| ⁸⁹Zr-girentuximab PET/CT | Baseline (screening) | Baseline (screening) |
| | Before dose 2 and dose 3 | Before dose 2 and dose 3 |
| | of ¹⁷⁷Lu-girentuximab | of ¹⁷⁷Lu-girentuximab |
| Whole body planar scan | 3 timepoints after dose 1 | 1 timepoint after all 3 doses |
| | of ¹⁷⁷Lu-girentuximab² | of ¹⁷⁷Lu-girentuximab³ |
| | 1 timepoint after dose 2 | |
| | and dose 3 of ¹⁷⁷Lu- | |
| | girentuximab³ | |
| SPECT/CT scan | 1 timepoint after all 3 | 1 timepoint after all 3 doses |
| | doses of ¹⁷⁷Lu-girentuximab³ | of ¹⁷⁷Lu-girentuximab³ |

¹The first 10 patients treated at the MTD who agree to additional imaging
²At 0-4 h, 48-72 h ± 6 h and 96-144 h ± 6 h post the first dose of ¹⁷⁷Lu-girentuximab

Proposed dosing schedule:
Day 1: ¹⁷⁷Lu-girentuximab
Day 2-15: Peposertib
Day 16-21: Treatment break
Day 22: ¹⁷⁷Lu-girentuximab
Day 23-37: Peposertib
Day 38-45: Treatment break
Day 46: ¹⁷⁷Lu-girentuximab
Day 47-61: Peposertib

### Inclusion Criteria:

Patients who meet all of the following criteria at Screening are eligible to participate in the study:
1 histologically confirmed renal cancer that has been treated with all previously recognized standard of care therapies.
2 at least one evaluable CAIX positive metastatic lesion in renal tissue as defined by RECIST 1.1 on zirconium-89 (⁸⁹Zr)-girentuximab PET/CT.
3 Age 18 years.
4 Karnofsky performance status ≥70.
5 Have adequate organ function at Screening:
   Bone marrow: Leukocytes ≥3,000/mL, Absolute neutrophil count ≥1500/mL, Platelets ≥100,000/mL, Hemoglobin ≥9 g/dL
   AST, ALT, and alkaline phosphatase ≤2.5×ULN, with the following exceptions:
      a) Patients with documented liver metastases: AST and/or ALT≤5×ULN
      b) Patients with documented liver or bone metastases: alkaline phosphatase ≤5×ULN
   Serum bilirubin ≤2×ULN (Patients with known Gilbert disease who have serum bilirubin level ≤3×ULN may be enrolled).
   INR and aPTT≤1.5×ULN (applies only to patients who are not receiving therapeutic anticoagulation; patients receiving therapeutic anticoagulation should be on a stable dose).
6 capacity to understand the study and be able and willing to comply with all protocol requirements.
7 ability to receive and maintain oral medication
8 will comply with the radiation protection guidelines (including hospital admissions and isolation) that are applied by the treating institution in order to protect their contacts and the general public.
9 will agree to practice adequate precautions to prevent pregnancy to avoid potential problems associated with radiation exposure to the unborn child (Refer to Clinical Trials Facilitation Group, 2020: Recommendations related to contraception and pregnancy testing in clinical trials Version 1.1, CTFG, 2020).

### Exclusion Criteria:

Patients who meet any of the following criteria are not eligible to participate in the study: prior treatment with 177Lu-girentuximab, known hypersensitivity to girentuximab or DOTA Linker; exposure to murine or chimeric antibodies within the last 5 years; receiving medications/herbal supplements known to be potent inhibitors or inducers of CYP3A or CYP2C19; previous administration of any radionuclide within 10 half-lives of the same; history of steroid requirement >10 mg daily prednisone in the past 2 years for autoimmune comorbidities; anti-cancer therapy within 2 weeks prior to enrolment; history of severe allergic, anaphylactic, or other hypersensitivity reactions to chimeric or humanized antibodies or fusion proteins; history of HIV infection, active or chronic hepatitis B or hepatitis C infection; significant cardiovascular disease, such as New York Heart Association cardiac disease (Class II or greater), myocardial infarction within the previous 3 months, unstable arrhythmias, unstable angina, or EF<50%, known coronary artery disease, congestive heart failure not meeting the above criteria must be on a stable medical regimen that is optimized in the opinion of the treating physician, in consultation with a cardiologist if appropriate; history of stroke or transient ischemic attack within 6 months prior to Cycle 1, Day 1; significant vascular disease (e.g., aortic aneurysm requiring surgical repair or recent peripheral arterial thrombosis) within 6 months prior to Cycle 1, Day 1; evidence of bleeding diathesis or significant coagulopathy (in the absence of therapeutic anticoagulation); clinical signs or symptoms of gastrointestinal obstruction or requirement for routine parenteral hydration, parenteral nutrition, or tube feeding; serious, non-healing or dehiscing wound, active ulcer, or untreated bone fracture; major surgery within 4 weeks prior to enrollment (biopsy or line placement can be performed up to 24 hours prior to enrollment); pregnant and lactating women.

### Investigational Product, Dosage and Mode of Administration:

¹⁷⁷Lu-TLX250, a chimeric monoclonal antibody (INN name: girentuximab (GTX), synonyms: cG250, TLX250) with specificity for the CAIX (carbonic anhydrase 9) antigen, radiolabelled with the positron emitting radio-metal luteium-177 via a DOTA linker.

### Duration of Treatment:

Approximately 9 or 36 weeks.

### Duration of Patient Study Participation:

Patients will be expected to participate in the study for up to 36 or 58 weeks

### Concomitant Therapy, Dosage and Mode of Administration:

Not applicable. Supportive care is permitted.

### Example 4: Clinical Trial of the Combination of ¹⁷⁷Lu-Rosopatamab and Peposertib in Patients with Prostate Specific Membrane Antigen (PSMA) Expressing Metastatic Castrate Resistant Prostate Cancer (mCRPC)

### Objectives

### Primary Objectives

### Phase I:

To determine the Maximum Tolerated Dose (MTD) of ¹⁷⁷Lu-rosopatamab (¹⁷⁷Lu-J591) in combination peposertib
Incidence of Treatment-emergent Adverse Events

### Phase II:

Objective response rates of ¹⁷⁷Lu-rosopatamab in combination peposertib

### Secondary Objectives:

Biochemical response as indicated by PSA levels, changes in tumor free circulating DNA tumor (ctDNA), alkaline phosphatase (ALP), CD4/CD8 subset analyses and lactate dehydrogenase (LDH) levels
Biological progression free survival
Radiographic Progression free survival
Overall survival
Best Overall Response Rate (BORR) as defined by RECIST criteria
Safety profile

### Methodology:

### Study Design Phase I

This is an open label, single-arm, randomized, parallel-group, multicenter dose finding study to evaluate ascending radioactive dose levels of ¹⁷⁷Lu-rosopatamab administered intravenously in combination with peposertib in relapsed/refractory patients with mCRPC expressing PSMA. ¹⁷⁷Lu-rosopatamab is given as 2 doses 14 days apart. Alternatively, ¹⁷⁷Lu-rosopatamab may be given as 2 doses 6 weeks apart.

Patients are treated in cohorts according to a 3+3 study design with dose levels as defined below.

Peposertib is administered at a dose of up to 400 mg BID daily from Day 1 until progression as determined by PET imaging.

The dose of 400 mg BID of peposertib will remain the same in the absence of dose limiting toxicities.

In the case of dose limiting toxicity with the initial dose, dose de-escalation may occur with the ¹⁷⁷Lu-rosopatamab and with the peposertib.

| | |
|---|---|
| Dose level −1 | 30 mCi/m2 + 300 mg peposertib |
| Dose level 1 | 30 mCi/m2 + 400 mg peposertib |
| Dose level 2 | 40 mCi/m2 + 400 mg peposertib |
| Dose level 3 | 45 mCi/m2 + 400 mg peposertib |

Alternatively, the dosing may be as follows:

| | |
|---|---|
| Dose level −1 | 30 mCi/m2 + 100 mg peposertib |
| Dose level 1 | 30 mCi/m2 + 175 mg peposertib |
| Dose level 2 | 30 mCi/m2 + 250 mg peposertib |
| Dose level 3 | 45 mCi/m2 + one step lower than no DLT |
| | dose peposertib |
| Dose level 4 | 45 mCi/m2 + no DLT dose peposertib |

Before moving to the next dose level a safety review committee will review the safety data from the current dose level for the first cycle.

### Study Design Phase 2:

32 patients will be treated with ¹⁷⁷Lu-rosopatamab at the recommended dosage determined in the phase 1 component of this study together with the recommended dose of peposertib until progression.

### Number of Patients (Planned):

### Phase 1: Dose-Finding: Estimated 12-18 Patients

Patients are treated in cohorts according to a 3+3 study design with at least four dose escalation cohorts and an optional dose de-escalation cohort.

### Phase 2: Preliminary Efficacy Determination: 32 Patients

Patients receive ¹⁷⁷Lu-rosopatamab and peposertib at the recommended dose determined in the phase 1 part of the study.

Proposed dosing schedule:
Day 1: ¹⁷⁷Lu-rosopatamab
Day 2-22: Peposertib
Day 23-60: Treatment break
Day 61: ¹⁷⁷Lu-rosopatamab
Day 62-82: Peposertib

### Inclusion Criteria:

Patients who meet all of the following criteria at Screening are eligible to participate in the study:
1 histologically confirmed mCRPC that has relapsed/is refractory to all standard of care therapies.
2 PSMA positivity as defined by imaging with Ga-PSMA PET imaging.
3 Age ≥18 years.
4 Karnofsky performance status ≥60.
5 have adequate organ function at Screening:
   Bone marrow: Leukocytes ≥3,000/mL, Absolute neutrophil count ≥1500/mL, Platelets ≥100,000/mL, Hemoglobin ≥9 g/dL
   Liver function: Total bilirubin ≤1.5×the upper limit of normal (ULN). For patients with known Gilbert's Syndrome ≤3×ULN is permitted; Alanine aminotransferase (ALT) or aspartate aminotransferase (AST)≤2.5×ULN
   Renal function serum/plasma creatinine ≤1.5×ULN or creatinine clearance ≥50 mL/min.
6 have the capacity to understand the study and be able and willing to comply with all protocol requirements.
7 have the capacity to understand the study and be able and willing to comply with all protocol requirements.
8 have the ability to receive and maintain oral medication
9 comply with the radiation protection guidelines (including hospital admissions and isolation) that are applied by the treating institution in order to protect their contacts and the general public.
10 agree to practice adequate precautions to prevent pregnancy in a partner to avoid potential problems associated with radiation exposure to the unborn child (Refer to Clinical Trials Facilitation Group, 2020: Recommendations related to contraception and pregnancy testing in clinical trials Version 1.1, CTFG, 2020),

### Exclusion Criteria:

Patients who meet any of the following criteria are not eligible to participate in the study: inability to undergo MRI or PET imaging; prior¹⁷⁷ Lutetium-based therapy (external radiation is permitted); receiving medications/herbal supplements known to be potent inhibitors or inducers of CYP3A or CYP2C19; history or evidence of delayed-type hypersensitivity (DTH)-dependent chronic infection (e.g. tuberculosis, systemic fungal or parasitic infection), potentially exacerbating under systemic corticoid therapy; known history of allergy to any excipient in the study medication or any other intravenously administered human proteins/peptides/antibodies; haemostaseologic conditions, precluding catheterisation or invasive procedures; chronically impaired renal function as indicated by creatinine clearance<45 mL/min or serum creatinine>1.5 ULN; any severe concomitant condition which makes it undesirable for the patient to participate in the study or which could jeopardize compliance with the protocol, in the opinion of the investigator; medically documented history of or active major depressive episode, bipolar disorder (I or II), obsessive-compulsive disorder, schizophrenia, a history of suicidal attempt or ideation, or homicidal ideation (e.g. risk of doing harm to self or others), or patients with active severe personality disorders; major trauma including major surgery (such as abdominal/cardiac/thoracic surgery) within 3 weeks of administration of study treatment; pregnancy or breast feeding; requirement of chronic administration of high dose corticosteroids or other immunosuppressant drugs; subjects must have been either off corticosteroids, or on a stable or decreasing dose s 4 mg daily dexamethasone (or equivalent) for 7 days prior to start of chemoradiotherapy. Limited or occasional use of corticosteroids to treat or prevent acute adverse reactions is not considered an exclusion criterion; HIV-positive participants on combination antiretroviral therapy are ineligible because of the potential for pharmacokinetic interactions with bavituximab. In addition, these participants are at increased risk of lethal infections when treated with marrow-suppressive therapy. Appropriate studies will be undertaken in participants receiving combination antiretroviral therapy when indicated; presence of active and uncontrolled infections or other severe concurrent disease, which, in the opinion of the investigator, would place the patient at undue risk or interfere with the study; concurrent malignancies unless the patient has been disease-free without intervention for at least 2 years serious, non-healing wound, ulcer, or bone fracture; requirement of concurrent use of other anti-cancer treatments or agents other than study medication; supportive care therapies are permitted; any recent live vaccination within 4 weeks prior to treatment or plan to receive vaccination during the study.

### Duration of Treatment:

Approximately 36 weeks.

### Duration of Patient Study Participation:

Patients will be expected to participate in the study for up to 58 weeks.

### Concomitant Therapy, Dosage and Mode of Administration:

Not applicable. Supportive care is permitted.

### Criteria for Evaluation:

### Phase 1 Primary Endpoints:

To determine the Maximum Tolerated Dose (MTD) of ¹⁷⁷Lu-rosopatamab in combination peposertib.
Treatment emergent adverse events (TEAE): Type according to MedDRA (Medical Dictionary for Regulatory Activities), frequency, severity according to NCI CTCAE V5.0, seriousness, and relationship of study treatment will be assessed. Laboratory abnormalities will be assessed according to the NCI CTCAE v.5.0Events.

### Phase 1 Secondary Endpoints

Whole body biodistribution and dosimetry (safety dosimetry)
Residence times for discernible organs (MBq*h)
Organ and whole body absorbed radiation doses (μGy/MBq)
Tumour dosimetry of ¹⁷⁷Lu-rosopatamab following systemic administration (therapeutic dosimetry)
Cmax (maximum activity concentration in tumour Bq/cm³)
tmax (time point of maximum activity concentration in tumour)
Tumour absorbed radiation dose (μGy/MBq)
Absorbed radiation doses (expressed as Gy/MBq of administered ¹⁷⁷Lu-rosopatamab) to kidneys, liver, lungs, spleen, bone/red marrow, gastrointestinal tract and below acceptable safe limits as defined by ARPANSA (see Radiation Risk Assessment).

### Phase 2 Primary Endpoints:

Overall Response Rates as defined by RECIST

### Phase 2 Secondary Endpoints:

Biochemical response as indicated by PSA levels, changes in tumor free circulating DNA tumor (ctDNA), alkaline phosphatase (ALP), CD4/CD8 subset analyses and lactate dehydrogenase (LDH) levels
Biological progression free survival
Radiographic Progression free survival
Overall survival
Best Overall Response Rate (BORR) as defined by RECIST criteria
Safety profile

### Example 5: Clinical Trial of the Combination of ¹⁷⁷Lu-Qirentuximab and Peposertib (M3814) with CAIX-Expressing Metastatic or Non-Resectable ccRCC

### Methodology:

All patients will undergo a ⁸⁹Zr-qirentuximab PET/CT scan prior to each ¹⁷⁷Lu-qirentuximab administration. To be eligible for the study, the baseline ⁸⁹Zr-TLX250CDx PET/CT must be positive, i.e. show uptake of ⁸⁹Zr-qirentuximab in at least 75% of the total lesion area or total lesion volume with intensity significantly greater than normal liver (i.e., standardized uptake value [SUV]ₘₐₓ at least 1.5 times SUV of normal liver).

For patients to be eligible for re-treatment with ¹⁷⁷Lu-qirentuximab and peposertib (i.e., Cycle 2 and Cycle 3, or later in case of response), a positive ⁸⁹Zr-qirentuximab PET/CT scan must also be obtained within 10 days prior to redosing.

Patients will undergo tumour assessments and evaluations according to RECIST 1.1 criteria. Contrast enhanced CT and/or MRI of the chest, abdomen and pelvis will be performed during screening (within 4 weeks prior to Cycle 1 Day 1), thereafter every 8 weeks for the first 6 months, every 12 weeks for the following 6 months. After the first year, patients should be scanned every six months. At these time points, patients in addition will undergo FDG-PET as clinically indicated.

Patients discontinuing study treatment for reasons other than progression of disease should follow this assessment schedule until disease progression, start of new anti-cancer therapy or lost to follow-up.

Part 1 will evaluate the combination of three different activities of ¹⁷⁷Lu-qirentuximab and three different dose levels of peposertib. Patients with CAIX positive renal cancer will be enrolled in a given dose level in cohorts of 2-4 patients (3 patients in the starting dose level). In the initial dose level patients will receive ¹⁷⁷Lu-qirentuximab at an activity of 1887 MBq (equivalent to a 1110 MBq/m² dose in a standard 1.7 m² adult individual)

Cycles 1-3 in combination with peposertib at a dose of 150 mg BID (D4-D21). Treatment cycles will have a fixed length of 84 days. Patients will be treated for a maximum of 3 administrations, or until clinically significant progression or unacceptable toxicity. Doses of ¹⁷⁷Lu-qirentuximab in Cycles 2 and 3 will be given at 75% of the activity of the prior cycle. Responding patients may receive treatment every 84 days beyond Cycle 3.

### Study Design Phase I:

¹⁷⁷Lu-qirentuximab will be administered at the following dose levels/activities:

| | |
|---|---|
| Dose level A | 1887 MBq |
| Dose level B | 2516 MBq |
| Dose level C | 3145 MBq |

Peposertib will be administered at the following dose levels:

| | |
|---|---|
| Dose level 1 | 100 mg peposertib BID (tablet) |
| Dose level 2 | 150 mg peposertib BID (tablet) |
| Dose level 3 | 200 mg peposertib BID (tablet) |

At the start of the escalation phase, patients will receive dose level A2, ie. 1887 MBq in combination with 150 mg BID peposertib.

After each completed dose level, the SRC will decide with support of the PO-BLRM outputs, which of the study drugs will be kept stable, escalated or de-escalated for the next dose level. Simultaneous escalation of both study drugs is not permitted.

Depending on the safety and/or efficacy data, the SRC may recommend testing additional dose schedules, e.g. delaying the start of peposertib to Day 7 or shortening the duration of peposertib administration.

Assessment of patient treatment, disease progression and inclusion and exclusion criteria are as for Example 3.

Proposed dosing schedule (cycles will be fixed 84 days in length)

### Cycles 1, 2 and 3:

Day −28 to −1: ¹⁷⁷Lu-girentuximab imaging+CT/MRI+/−FDG-PET
Day 1: ¹⁷⁷Lu-girentuximab injection
Day 4-21: Peposertib PO BID
Day 22 to 84: Treatment break

### Investigational Product, Dosage and Mode of Administration:

¹⁷⁷Lu-TLX250, a chimeric monoclonal antibody (INN name: girentuximab (GTX), synonyms: cG250, TLX250; ¹⁷⁷Lu-DOTA-girentuximab) with specificity for the CAIX (carbonic anhydrase 9) antigen, radiolabelled with the positron emitting radio-metal luteium-177 via a DOTA linker.

It contains 1700-4080 MBq of ¹⁷⁷Lu-TLX250 in 10 mL, which is sufficient to draw up the proposed single clinical dose of up to 2405 MBq/m2 of ¹⁷⁷Lu-TLX250, with a total antibody mass dose of 10 mg at the time of administration.

The non-radioactive part is the immunoconjugate DOTA-girentuximab, with a total antibody dose of 10 mg (i.e., there will be no unconjugated girentuximab). ¹⁷⁷Lu-TLX250 (¹⁷⁷Lu-DOTA-girentuximab) is intended for IV administration via slow IV push. The final ¹⁷⁷Lu-girentuximab product contains 1700-4080 MBq of 177Lu-girentuximab in 10 mL, which is sufficient to draw up the patient dose of up to 2405 MBq/m2 of 177Lu-girentuximab, with a total antibody mass dose of 10 mg at the time of administration. The injectate has high radiochemical purity (greater than or equal to 90%) and includes less than 10% of both 177Lu and 177Lu-DOTA.

The intended dosing schedule is planned for up to 3 repeat doses, administered on every 84 days (Day 1 of Cycles 1 to 3), with subsequent doses at 75% of the previous dose. This dosing regime is based on previous clinical studies in which the MTD was determined to be 2405 MBq/m² where follow on dosing at 75% of the previous dose was well tolerated. While in total, up to 3 doses are intended to be administered, each of the 3 doses can be considered as a single-dose administration, as the spacing between doses is greater than 4 weeks.

No accumulation of chemical toxicity related to DOTA-girentuximab can be expected from 12-14 weeks of spaced dosing.

Peposertib (M3814), with the chemical name (S)-[2-chloro-4-fluoro-5-(7-morpholin-4-ylquinazolin-4-yl)-phenyl]-(6-methoxy-pyridazin-3-yl)-methanol, is a potent and selective small-molecule adenosine triphosphate-competitive inhibitor of DNA-PK that targets tumor cell growth and survival by inhibiting a critical DNA damage repair mechanism in solid and hematological malignancies. For this clinical study, peposertib film-coated tablets containing 50 mg of drug substance are available. Peposertib film-coated tablets represent formulations for oral administration.

⁸⁹Zr-girentuximab is an investigational agent, supplied as a ready to inject solution. During screening, a non-therapeutic assessment will be mandated for inclusion in the study, involving diagnostic administration of ⁸⁹Zr-girentuximab, followed by a PET CT scan 4 to 7 days post ⁸⁹Zr-girentuximab. ⁸⁹Zr-girentuximab is a chimeric monoclonal antibody (INN name: girentuximab (GTX), synonyms: cG250, TLX250) with specificity for the CAIX (carbonic anhydrase 9) antigen, radiolabelled with the positron emitting radio-metal zirconium-89 (⁸⁹Zr via a NSuc-DFO-TFPester (DFO-TFP), linked to lysine residues of GTX, to yield ⁸⁹Zr-DFOTFP-GTX.

A single administration of 37 MBq (±10%)⁸⁹Zr-TLX250, containing a mass dose of 10 mg of girentuximab will be administered by slow intravenous (IV) administration over a minimum of 3 minutes. Before and after administration, safety evaluations will be made. ⁸⁹Zr-girentuximab is formulated as a solution for intravenous administration in glass vials at the nominal dosage strength 37 MBq (±10%) for single intravenous use. The selected dose level is based on previous safety, biodistribution, and dosimetry findings of the Phase I study.

The ⁸⁹Zr-girentuximab drug product is manufactured as “ready-to-use”. No dietary constrictions prior to dosing are necessary. Whole body PET/CT scans (skull base to mid-thigh) will be acquired using 6-8 bed positions with 5-10 minutes acquisition time per bed position at a single time point 4-7 days post administration of 89Zrgirentuximab using low dose CT. ⁸⁹Zr-girentuximab standard uptake values (SUVs) will be determined for each tumor lesion. Scans will be performed at baseline and prior to subsequent 177Lu-girentuximab doses (e.g., at approximately C2D1 and C3D1)

### Duration of Treatment:

Approximately 7 months. Follow-up will be approximately 6 months after completion of dosing.

It will be understood that the invention disclosed and defined in this specification extends to all alternative combinations of two or more of the individual features mentioned or evident from the text or drawings. All of these different combinations constitute various alternative aspects of the invention.

1. A method for treating a disease or disorder characterised by aberrant cell growth and function in a subject, the method comprising administering to a subject in need thereof, a combination therapy comprising:
i) a DNA-PK inhibitor (DNA-PKi);
ii) a molecular targeted radiotherapeutic capable of cellular internalisation and/or retention in the circulation of the subject;
wherein the radiotherapeutic comprises a radionuclide that is a beta emitter;
thereby treating the disease or disorder characterised by aberrant cell growth and function in the subject.

2. A method for treating a disease or disorder characterised by aberrant cell growth and function in a subject, the method comprising administering to a subject in need thereof, a combination therapy comprising:
i) (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof;
ii) a molecular targeted radiotherapeutic capable of cellular internalisation and/or retention in the circulation of the subject;
thereby treating the disease or disorder characterised by aberrant cell growth and function in the subject.

3. The method of claims 1 or 2, wherein the molecular targeted radiotherapeutic is a radioimmunoconjugate, preferably, an antibody or antigen binding fragment thereof for binding to an antigen associated with the disease or disorder for which treatment is required and to which is conjugated a radionuclide.

4. The method of any one of claims 1 to 3, wherein the molecular targeted radiotherapeutic is an antibody for binding to an antigen associated with the disease or disorder for which treatment is required, wherein the antibody is conjugated to a radionuclide, preferably wherein the antibody is an immunoglobulin selected from IgG1, IgG2, IgG3, and IgG4, particularly an antibody that is predominantly subject to hepatic clearance.

5. The method of claim 4, wherein the disease or disorder is a cancer, and the molecular targeted radiotherapeutic comprises an antibody, or antigen binding fragment thereof, for binding to a tumour-associated or tumour-specific antigen expressed by the cancer, optionally wherein the cancer is a metastatic cancer.

6. The method of claim 4, wherein the disease or disorder to a non-cancerous proliferative cell disorder and the molecular targeted radiotherapeutic comprises an antibody, or antigen binding fragment thereof, for binding to an antigen expressed by the proliferating cells.

7. The method of claim 5, wherein the disease or disorder to be treated is a cancer characterised by the expression of carbonic anhydrase IX (CAIX) and the molecular targeted radiotherapeutic comprises an antibody or antigen binding fragment thereof capable of specifically binding to CAIX.

8. The method of claim 5, wherein the disease or disorder to be treated is a cancer characterised by the expression of prostate specific membrane antigen (PSMA) and the molecular targeted radiotherapeutic comprises an antibody or antigen binding fragment thereof capable of specifically binding to PSMA.

9. The method of any one of claims 2 to 8, wherein the radionuclide is an alpha emitter, preferably selected from the group consisting of Astatine-²¹¹ (²¹¹At), Bismuth-²¹² (²¹²Bi), Bismuth-²¹³ (²¹³Bi), Actinium-²²⁵ (²²⁵Ac), Radium-²²³ (²²³Ra), Lead-²¹² (²¹²Pb), Thorium-²²⁷ (²²⁷Th), and Terbium-¹⁴⁹ (¹⁴⁹Tb), more preferably wherein the radionuclide is Astatine-²¹¹ (²¹¹At) or Actinium-²²⁵ (²²⁵Ac).

10. The method of any one of claims 1 to 8, wherein the radionuclide is beta or beta/gamma emitter, preferably selected from the group consisting of: Lutetium-¹⁷⁷ (¹⁷⁷Lu), Yttrium-⁹⁰ (⁹⁰Y), Iodine-¹³¹ (¹³¹I), Samarium-¹⁵³ (¹⁵³Sm), Holmium-¹⁶⁶ (¹⁶⁶Ho), Rhenium-¹⁸⁶ (¹⁸⁶Re), or Rhenium-¹⁸⁸ (¹⁸⁸Re), more preferably wherein the radionuclide is Lutetium-¹⁷⁷ (¹⁷⁷Lu) or Rhenium-¹⁸⁸ (¹⁸⁸Re).

11. A method for treating a cancer characterised by the expression of CAIX, the method comprising administering to a subject in need thereof, a combination therapy comprising:
i) (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof;
ii) an antibody or antigen binding fragment thereof for binding to CAIX, wherein the antibody or fragment thereof is conjugated to a radionuclide for delivering a radiotherapeutic dose to the cancer;
thereby treating the cancer in the subject.

12. The method of any claim 11, wherein the antibody or antigen binding fragment for binding to CAIX comprises a G250 antibody or is an antibody comprising an antigen binding domain as defined in Table 2 herein.

13. The method of claim 11, wherein the antigen binding domain comprises:
a) a heavy chain variable region comprising a CDR1 comprising an amino acid sequence as set forth in any of SEQ ID NOs: 49, 65, 81, 97, and 113, a CDR2 comprising an amino acid sequence as set forth in any of SEQ ID NOs: 50, 66, 82, 98 and 114; and a CDR3 comprising an amino acid sequence as set forth in any of SEQ ID NOs: 51, 67, 83, 99 and 115; and
b) a light chain variable region comprising a CDR1 comprising an amino acid sequence as set forth in any of SEQ ID NOs: 129, 145, 161, 177, 193 and 209, a CDR2 comprising an amino acid sequence as set forth in any of SEQ ID NOs: 130, 146, 162, 178, 194 and 210; and a CDR3 comprising an amino acid sequence as set forth in any of SEQ ID NOs: 131, 147, 163, 179, 195 and 211.

14. The method of claim 11, wherein the antigen binding domain comprises a heavy chain variable region comprising an amino acid sequence at least 80% identical to the amino acid sequence as defined in any one of SEQ ID NOs: 52, 68, 84, 100 and 116 and a light chain variable region comprising an amino acid sequence at least 80% identical to the amino acid sequence as defined in any of SEQ ID NOs: 132, 148, 164, 180, 196 and 212.

15. The method of claim 11, wherein the antibody comprises the amino acid sequences set forth in any one of SEQ ID NOs: 225 to 228, preferably in combination with SEQ ID NO: 229; most preferably comprising the amino acid sequences set forth in SEQ ID NO: 231 and 234.

16. The method of any one of claims 11 to 15, wherein the cancer characterised by the expression of CAIX is selected from the group consisting of: renal cell carcinoma (including clear renal cell carcinoma), colon cancer, breast cancer, lung cancer, cervical cancer and melanoma, preferably wherein the cancer is renal cancer.

17. The method of claim 16, wherein the cancer is a metastatic cancer, optionally metastatic renal cell carcinoma.

18. A method for treating a cancer characterised by the expression of PSMA, the method comprising administering to a subject in need thereof, a combination therapy comprising:
i) (S)-[2-chloro-4-fluoro-5-(7-morpholin-4-yl-quinazolin-4-yl)-phenyl]-(6-methoxypyridazin-3-yl)-methanol (M3814) or a pharmaceutically acceptable salt thereof;
ii) an antibody or antigen binding fragment thereof for binding to PSMA, wherein the antibody or fragment thereof is conjugated to a radionuclide for delivering a radiotherapeutic dose to the cancer;
thereby treating the cancer in the subject.

19. The method of claim 18, wherein the antibody or antigen binding fragment for binding to PSMA comprises a J591 antibody, or variant or humanised form thereof, or an antibody comprising an antigen binding domain as defined in Table 1 herein.

20. The method of claim 18, wherein the antibody or antigen binding fragment comprises
a) a heavy chain variable region comprising a CDR1 comprising an amino acid sequence as set forth in any of SEQ ID NOs: 1, 17 or 244, a CDR2 comprising an amino acid sequence as set forth in any of SEQ ID NOs: 2 or 18; and a CDR3 comprising an amino acid sequence as set forth in any of SEQ ID NOs: 3 or 19; and
b) a light chain variable region comprising a CDR1 comprising an amino acid sequence as set forth in SEQ ID NO: 33, a CDR2 comprising an amino acid sequence as set forth in SEQ ID NO: 34; and a CDR3 comprising an amino acid sequence as set forth in SEQ ID NO: 35.

21. The method of claim 18, wherein the antigen binding domain comprises a heavy chain variable region comprising an amino acid sequence at least 80% identical to the amino acid sequence as defined in any one of SEQ ID NOs: 4 and 20 and a light chain variable region comprising an amino acid sequence at least 80% identical to the amino acid sequence as defined in SEQ ID NO: 36.

22. The method of claim 18, wherein the antibody comprises the amino acid sequences set forth in any one of SEQ ID NOs: 239 to 242, preferably comprising the amino acid sequence of SEQ ID NO: 239 and 243.

23. The method of any one of claims 18 to 22, wherein the cancer characterised by the expression of PSMA is selected from the group consisting of: prostate cancer, bladder cancer, testicular-embryonal cancer, neuroendocrine cancer, renal cell carcinoma, and breast cancer, preferably wherein the cancer is prostate cancer.

24. The method of claim 23, wherein the cancer is a metastatic prostate cancer, optionally metastatic castration-resistant prostate cancer (mCRPC).

25. The method of any one of claims 11 to 24, wherein the radionuclide is an alpha emitter, preferably selected from the group consisting of Astatine-²¹¹ (²¹¹At), Bismuth-²¹² (²¹²Bi), Bismuth-²¹³ (²¹³Bi), Actinium-²²⁵ (²²⁵Ac), Radium-²²³ (²²³Ra), Lead-²¹² (²¹²Pb), Thorium-²²⁷ (²²⁷Th), and Terbium-¹⁴⁹ (¹⁴⁹Tb), more preferably wherein the radionuclide is Astatine-²¹¹ (²¹¹At) or Actinium-²²⁵ (²²⁵Ac).

26. The method of any one of claims 11 to 24, wherein the radionuclide is beta or beta/gamma emitter, preferably selected from the group consisting of: Lutetium-¹⁷⁷ (¹⁷⁷Lu), Yttrium-⁹⁰ (⁹⁰Y), Iodine-¹³¹ (¹³¹I), Samarium-¹⁵³ (¹⁵³Sm), Holmium-¹⁶⁶ (¹⁶⁶Ho), Rhenium-¹⁸⁶ (¹⁸⁶Re), and Rhenium-¹⁸⁸ (¹⁸⁸Re).

27. The method of claim 26, wherein the radionuclide is Lutetium-¹⁷⁷ (¹⁷⁷Lu) or Rhenium-¹⁸⁸ (¹⁸⁸Re).

28. The method of any one of claims 1 to 27, wherein the molecular targeted radiotherapeutic and the DNA-PKi are administered sequentially in either order, or simultaneously.

29. The method of any one of claims 1 to 27, wherein the DNA-PKi, preferably M3814, is administered subsequent to the administration of the molecular targeted radiotherapeutic.

30. The method of claim 29, wherein the DNA-PKi, preferably M3814, is administered at least one day, at least 2 days, at least 3 days, at least 4 days, at least 5 days, at least 6 days, or at least 7 days or more, following administration of the molecular targeted radiotherapeutic.

31. The method of claim 25, wherein the DNA-PKi, preferably M3814, is administered no more than 7 days, no more than 6 days, no more than 5 days, no more than 4 days, no more than 3 days, no more than 2 days or no more than 1 day following administration of the molecular targeted radiotherapeutic.

32. The method of claim 29, wherein the DNA-PKi, preferably M3814, is administered within 24 hours following administration of the molecular targeted radiotherapeutic.

33. The method of any one of claims 1 to 32, wherein the method comprises administration of a single dose only of the molecular targeted radiotherapeutic, to treat the disease or disorder characterised by aberrant cell growth or function in the subject.

34. The method of any one of claims 1 to 32, wherein the method comprises administration of one or more treatment cycles, wherein each treatment cycle comprises administration of the molecular targeted radiotherapeutic, followed by at least 7 days, at least 14 days, at least 21 days or more, of administration of the DNA-PKi, preferably M3814.

35. The method of claim 34, wherein the method comprises more than one treatment cycle, and wherein there is a treatment break between treatment cycles, preferably wherein the treatment break is for a period of at least 7 days, at least 14 days, at least 21 days, at least 28 days, at least 35 days, at least 42 days, at least 49 days, at least 56 days, at least 63 days or more; more preferably wherein the treatment break is for no more than 100 days.

36. The method of any one of claims 1 to 35, wherein the dose of the radiotherapeutic is at least about 25%, at least about 30%, at least about 35%, at least about 40%, at least about 45% or at least about 50% lower than the therapeutic dose required for monotherapy with the radiotherapeutic.

37. The method of any one of claims 1 to 36, wherein the dose of the DNA-PKi administered, preferably M3814, is a dose level below the maximum tolerated dose level, optionally at a dose of up to 90%, 85%, 80%, 75%, 60%, 65%, 60% or 55% of the maximum tolerated dose level, and/or at least 10%, or 20%, 30%, 40% or 50% of the maximum tolerated dose level of the combination.

38. The method of any one of claims 1 to 37, wherein the method further comprises administration of iii) an additional anti-cancer therapy selected from the group consisting of: an immune check-point modulator, a chemotherapeutic, and a radiation sensitiser.

39. The method of claim 38, wherein the additional anti-cancer therapy comprises an immune checkpoint modulator.

40. The method of claim 39, wherein the immune checkpoint modulator is an immune checkpoint inhibitor selected from: an inhibitor of PD-1, PD-L1 and CTLA-4 or any other immune checkpoint inhibitor described herein.

41. The method of claim 40, wherein the immune checkpoint inhibitor is an inhibitor of PD-1 selected from: pembrolizumab nivolumab, cemiplimab, spartalizumab, camrelizumab, sintilimab, tislelizuma, toripalimab, dostarlimab, INCMGA00012, AMP-224 and AMP-514.

42. The method of claim 40, wherein the immune checkpoint inhibitor is an inhibitor of PD-L1 selected from: atezolizumab, durvalumab, KN035, CK-301, AUNP12, CA-170, and BMS-986189.

43. The method of claim 40, wherein the immune checkpoint inhibitor is an inhibitor of CTLA-4, selected from: ipilimumab and tremelimumab.
